(19) United States
(12) Patent Application Publication
(10) Pub. No.: US 2004/0063959 A1
(43) Pub. Date: Apr. 1, 2004

(54) Esters and amides as PLA2 inhibitors

(76) Inventors: Naoki Fukami, Ibaraki (JP); Seiji Yoshimura, Tsukuba-shi (JP); Keisuke Imai, Tsukuba-shi (JP); Keiji Hemmi, Tsukuba-shi (JP); Mitsue Hemmi, Tsukuba-shi (JP); Keiichiro Hemmi, Tsukuba-shi (JP); Yusuke Hemmi, Tsukuba-shi (JP)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/665,466

(22) Filed: Sep. 22, 2003

Related U.S. Application Data
(63) Continuation of application No. 10/138,637, filed on May 6, 2002.
(63) Continuation of application No. 08/981,830, filed on Apr. 20, 1998, now abandoned.
Application No. 08/981,830 is a 371 of international application No. PCT/JP96/01995, filed on Jul. 18, 1996.

(30) Foreign Application Priority Data
Jul. 24, 1995 (GB) 9515162.7
May 14, 1996 (AU) PN 9835

(51) Int. Cl. C07D 233/54; C07D 213/55; C07D 233/66; C07D 209/18

(52) U.S. Cl. 546/335; 546/336; 548/338.1; 548/495; 554/51

(57) ABSTRACT

The present invention relates to a novel fatty acid derivative of the following formula:

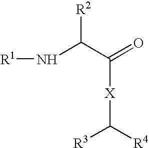

wherein R¹ is acyl group;

R² is acyl(lower)alkyl;

R³ is hydrogen, aryl(lower)alkyl, etc;

R⁴ is acyl(lower)alkyl; and

X is —O—, —NH— or

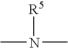

[wherein R⁵ is lower alkyl, etc];

and a pharmaceutically acceptable salt thereof, which is useful as a medicament; the processes for the preparation of said fatty acid derivative or a salt thereof;

a pharmaceutical composition comprising said fatty acid derivative or a pharmaceutically acceptable salt thereof; etc.

### TECHNICAL FIELD

The present invention relates to a novel fatty acid derivative and a pharmaceutically acceptable salt thereof which are useful as a medicament.

### BACKGROUND ART

A phospholipase A₂ inhibitor having the structure of that of the present invention has not been known.

### DISCLOSURE OF INVENTION

The present invention relates to novel fatty acid derivative and a pharmaceutically acceptable salt thereof, which are phospholipase A₂ inhibitors and are useful for the prevention and/or the treatment of pancreatitis, hepatitis, chronic renal failure, etc; shock (e.g. endotoxin shock, gram-negative septic shock, etc), arthritis (e.g. rheumatoid arthritis, osteoarthritis, etc), respiratory disease (e.g. bronchial asthma, bronchitis, adult respiratory distress syndrome, etc), heart disease (e.g. myocardial ischemia, etc), allergic disease, thrombosis, arteriosclerosis, pain, autoimmune disease, dermal disease (e.g. atopic dermatitis, psoriasis, contact dermatitis, etc), inflammatory bowel disease (e.g. Crohn's disease, ulcerative colitis, etc), ophthalmic disease (e.g. allergic ophthalmic disease, inflammatory ophthalmic disease, etc), nasal diseases (e.g. allergic rhinitis, etc), gout, trauma induced inflammation (e.g. spinal cord injury, etc), liver diseases (e.g. cirrhosis, hepatitis, etc), or the like; to a process for preparation thereof, to a pharmaceutical composition comprising the same, and to a method for using the same therapeutically in human being and animals for the prevention and/or treatment of the aforesaid diseases.

The object fatty acid derivative can be represented by the following formula (I)

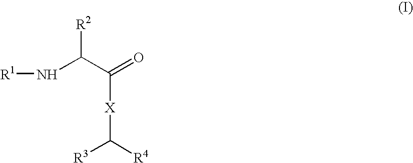

wherein R¹ is acyl group,

R² is acyl(lower)alkyl,

R³ is hydrogen, aryl(lower)alkyl which may have one or more suitable substituent(s), aryl(higher)alkyl which may have one or more suitable substituent(s), heterocyclic(lower)alkyl which may have one or more suitable substituent(s), higher alkoxy(lower)alkyl, lower alkyl, or higher alkyl,

R⁴ is acyl(lower)alkyl, and

X is —O—, —NH— or

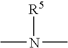

[wherein R⁵ is lower alkyl, [cyclo(lower)alkyl](lower)alkyl, aryl(lower)alkyl, or heterocyclic(lower)alkyl],

with proviso that X is

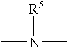

(wherein R⁵ is as defined above), when R³ is lower alkyl or higher alkyl.

It is to be noted the object compound (I) may include one or more stereoisomers due to asymmetric carbon atom(s) and double bond, and all of such isomers and a mixture thereof are included within the scope of the present invention.

It is further to be noted isomerization or rearrangement of the object compound (I) may occur due to the effect of the light, acid, base or the like, and the compound obtained as the result of said isomerization or rearrangement is also included within the scope of the present invention.

It is also to be noted that the solvating form of the compound (I) (e.g. hydrate, etc) and any form of the crystal of the compound (I) are included within the scope of the present invention.

The object compound (I) or a salt thereof can be prepared according to the following reaction schemes.

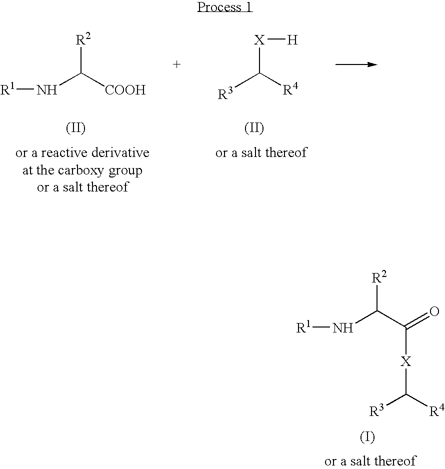

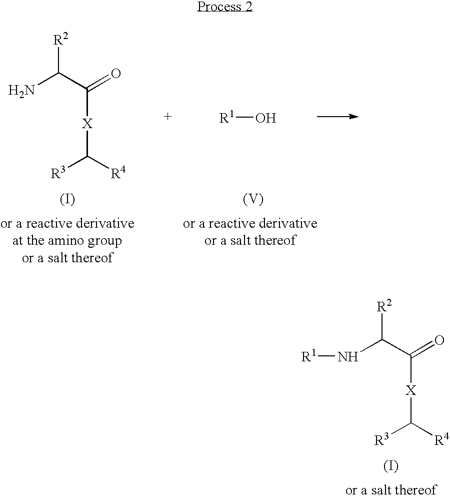

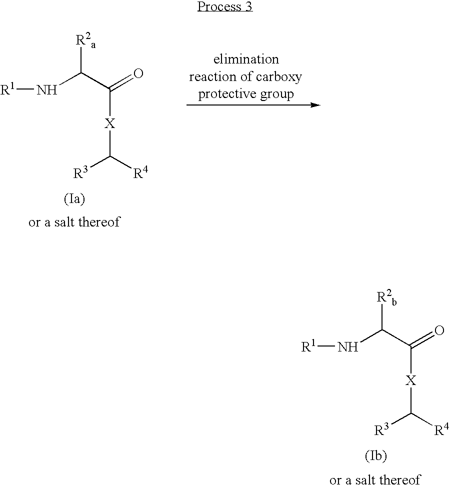

wherein R¹, R², R³, R⁴ and X are each as defined above,

Rₐ² is protected carboxy(lower)alkyl,

R_{b}² is carboxy(lower)alkyl.

The starting compound (IV) or a salt thereof can be prepared according to the following reaction scheme.

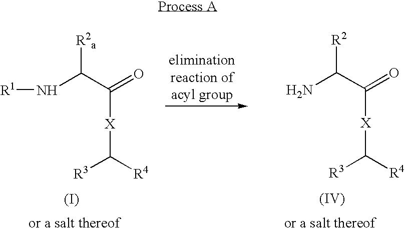

wherein R¹, R², R³, R⁴ and X are each as defined above.

Among the starting compounds, there are some novel compounds. They can be prepared according to the methods as described in Preparations in the present specification or the conventional manners in this field of the art.

Suitable pharmaceutically acceptable salts of the object compound (I) are conventional ones and include a metal salt such as an alkali metal salt (e.g. sodium salt, potassium salt, etc) and an alkaline earth metal salt (e.g. calcium salt, magnesium salt, etc), an ammonium salt, an organic base salt (e.g. trimethylamine salt, triethylamine salt, pyridine salt, picoline salt, dicyclohexylamine salt, N,N′-dibenzylethylenediamine salt, etc), an organic acid salt (e.g. acetate, trifluoroacetate, maleate, tartrate, fumarate, methanesulfonate, benzenesulfonate, formate, toluenesulfonate, etc), an inorganic acid salt (e.g. hydrochloride, hydrobromide, hydriodide, sulfate, phosphate, etc), a salt with an amino acid (e.g. arginine, aspartic acid, glutamic acid, etc), and the like.

In the above and following descriptions of the present specification, suitable examples and illustrations of the various definitions which the present invention includes within the scope thereof are explained in detail as follows.

The term “lower” is intended to mean 1 to 6 carbon atom(s) unless otherwise indicated.

The term “higher” is intended to mean 7 to 20 carbon atoms unless otherwise indicated.

Suitable example of “lower alkyl” and “lower alkyl” moiety in the terms used in the present specification may include straight or branched one such as methyl, ethyl, propyl, isopropyl, butyl, isobutyl, t-butyl, pentyl, isopentyl, hexyl or the like.

Suitable “lower alkenyl” and “lower alkenyl” moiety in the terms used in the present specification may include vinyl, 1-(or 2-)propenyl, 1-(or 2- or 3-)butenyl, 1-(or 2- or 3- or 4-)pentenyl, 1-(or 2- or 3- or 4- or 5-)hexenyl, methylvinyl, ethylvinyl, 1-(or 2- or 3-)methyl-1-(or 2-)propenyl, 1-(or 2- or 3-)ethyl-1-(or 2-)propenyl, 1-(or 2- or 3- or 4-)methyl-1-(or 2- or 3-)butenyl or the like, in which the preferred one may be (C₂-C₄)alkenyl.

Suitable “higher alkyl” and “higher alkyl” moiety in the terms used in the present specification may include straight or branched one such as heptyl, 2-methylheptyl, octyl, nonyl, decyl, undecyl, dodecyl, tridecyl, 11-methyldodecyl, 12-methyltridecyl, tetradecyl, pentadecyl, hexadecyl, heptadecyl, octadecyl, nonadecyl, icosyl or the like, in which the preferred one may be (C₇-C₁₆)alkyl, and the more preferred one may be heptyl, octyl, nonyl, decyl, or tridecyl.

Suitable “halogen” may include fluorine, chlorine, bromine, iodine, in which more preferable one may be chlorine.

Suitable “aryl” and “aryl” moiety in the terms used in the present specification may include phenyl, naphthyl and the like.

Suitable “acyl group” and “acyl” moiety in the terms used in the present specification may be aliphatic acyl, aromatic acyl, heterocyclic acyl, arylaliphatic acyl and heterocyclic-aliphatic acyl derived from carboxylic acid, carbonic acid, carbamic acid, sulfonic acid, and the like.

Suitable example of the “acyl group” thus explained may be:

(1) lower alkanoyl [e.g. formyl, acetyl, propionyl, butyryl, isobutyryl, valeryl, hexanoyl, pivaloyl, etc] which may have one or more (preferably 1 to 3) suitable substituent(s) such as halogen (e.g. fluoro, chloro, bromo, iodo); hydroxy; lower alkoxy (e.g. methoxy, ethoxy, propoxy, butoxy, t-butoxy, pentyloxy, hexyloxy, etc); amino; protected amino, preferably, acylamino such as lower alkoxycarbonylamino (e.g. methoxycarbonylamino, ethoxycarbonylamino, propoxycarbonylamino, butoxycarbonylamino, t-butoxycarbonylamino, pentyloxycarbonylamino, hexyloxycarbonylamino, etc); or the like; di(lower) alkylamino (e.g. dimethylamino, N-methylethylamino, diethylamino, N-propylbutylamino, dipentylamino, dihexylamino, etc); lower alkoxyimino (e.g. methoxyimino, ethoxyimino, propoxyimino, butoxyimino, t-butoxyimino, pentyloxyimino, hexyloxyimino, etc); ar(lower)alkoxyimino such as phenyl(lower)alkoxyimino (e.g. benzyloxyimino, phenethyloxyimino, benzhydryloxyimino, etc); or the like;

(2) higher alkanoyl [e.g. heptanoyl, octanoyl, nonanoyl, decanoyl, undecanoyl, lauroyl, tridecanoyl, myristoyl, pentadecanoyl, palmitoyl, 14-methylpentadecanoyl, 15-methylhexadecanoyl, 10, 12-dimethyltetradecanoyl, heptadecancyl, stearoyl, nonadecanoyl, icosanoyl, etc) which may have one or more (preferably 1 to 3) suitable substituent(s) as exemplified for those of “lower alkanoyl”;

(3) lower alkenoyl (e.g. acryloyl, crotonoyl, isocrotonoyl, methacryloyl, 3-pentenoyl, 2,4-pentadienoyl, 5-hexenoyl, 2,4-hexadienoyl, etc] which may have one or more (preferably 1 to 3) suitable substituent(s) as exemplified for those of “lower alkanoyl”;

(4) higher alkenoyl [e.g. 4-heptenoyl, 3-octenoyl, 3,6-decadienoyl, 3,7,11-trimethyl-2,6,10-dodecatrienoyl, 4,10-heptadecadienoyl, etc] which may have one or more (preferably 1 to 3) suitable substituent(s) as exemplified for those of “lower alkanoyl”;

(5) protected carboxy, in which the preferred one may be esterified carboxy such as lower alkoxycarbonyl (e.g. methoxycarbonyl, ethoxycarbonyl, propoxycarbonyl, butoxycarbonyl, t-butoxycarbonyl, pentyloxycarbonyl, hexyloxycarbonyl, etc),

halo(lower)alkoxycarbonyl [e.g. (chloromethoxy)carbonyl, (2,2,2-trichloroethoxy)carbonyl, (2,2,2-trifluoroethoxy)carbonyl, (2-chloropropoxy)carbonyl, 1-fluoro-4-bromobutoxy)carbonyl, (4-chloropentyloxy)-carbonyl, (6-chlorohexyloxy)carbonyl, etc],

higher alkoxycarbonyl [e.g. heptyloxycarbonyl, octyloxycarbonyl, 2-ethylhexyloxycarbonyl, nonyloxycarbonyl, decyloxycarbonyl, 3,7-dimethyloctyloxycarbonyl, undecyloxycarbonyl, dodecyloxycarbonyl, tridecyloxycarbonyl, tetradecyloxycarbonyl, pentadecyloxycarbonyl, 3-methyl-10-ethyldodecyloxycarbonyl, hexadecyloxycarbonyl, heptadecyloxycarbonyl, octadecyloxycarbonyl, nonadecyloxycarbonyl, icosyloxycarbonyl, etc],

aryloxycarbonyl [e.g. phenoxycarbonyl, naphthyloxycarbonyl, etc],

aryl(lower)alkoxycarbonyl which may have one or more (preferably 1 to 3) suitable substituent(s) such as phenyl(lower)alkoxycarbonyl which may have nitro or lower alkoxy [e.g. benzyloxycarbonyl, phenethyloxycarbonyl, p-nitrobenzyloxycarbonyl, p-methoxybenzyloxycarbonyl, etc], or the like;

(6) carboxy;

(7) lower alkylsulfonyl [e.g. methylsulfonyl, ethylsulfonyl, propylsulfonyl, isopropylsulfonyl, pentylsulfonyl, butylsulfonyl, etc];

(8) arylsulfonyl [e.g. phenylsulfonyl, 1-(or 2-)naphthylsulfonyl, etc] which may have one or more (preferably 1 to 3) suitable substituent(s) such as lower alkyl, di(lower)alkylamino, lower alkylamino (e.g. methylamino, ethylamino, propylamino, butylamino, t-butylamino, pentylamino, hexylamino, etc), or the like;

(9) aryl(lower)alkylsulfonyl such as phenyl(lower)alkylsulfonyl [e.g. benzylsulfonyl, phenethylsulfonyl, benzhydrylsulfonyl, etc], or the like;

(10) aryl(lower)alkanoyl such as phenyl(lower)alkanoyl or naphthyl(lower)alkanoyl [e.g. benzoyl, naphthoyl (e.g. 1-naphthoyl, 2-naphthoyl, etc), 2-phenylacetyl, 2-phenylpropionyl, 4-(1-naphthyl)butyryl, 3-phenylvaleryl, 2,5-diphenylhexanoyl, etc], each of which may have one or more (preferably 1 to 3) suitable substituent(s) such as lower alkoxy, aryl (e.g. phenyl, naphthyl, anthryl, etc), carboxy(lower)alkyl (e.g. carboxymethyl, 2-carboxyethyl, 1-carboxypropyl, 4-carboxybutyl, 3-carboxypentyl, 6-carboxyhexyl, etc), protected carboxy(lower)alkyl (e.g. methoxycarbonylmethyl, 2-methoxycarbonylethyl, 2-(t-butoxycarbonyl)ethyl, etc) which may be substituted by aryl (e.g. phenyl, naphthyl, etc), protected carboxy(lower)alkenyl (e.g. 2-methoxycarbonylvinyl, etc), amidated carboxy(lower)alkyl (e.g. 2-carbamoylethyl, etc), aryl(lower)alkyl (e.g. benzyl, phenethyl, etc) which may have one or more suitable substituent(s), or the like;

(11) aryl(lower)alkenoyl (e.g. 3-phenylacryloyl, 2-phenylacryloyl, 2-naphthylacryloyl, 3-phenylcrotonoyl, 4-phenylisocrotonoyl, 2-benzylacryloyl, 5-phenyl-3-pentenoyl, 3-naphthyl-2,4-pentadienoyl, 2-phenyl-5-hexenoyl, 6-phenyl-2,4-hexadienoyl, etc);

(12) heterocyclic(lower)alkanoyl which may have one or more (preferably 1 to 3) suitable substituent(s) such as lower alkyl, aryl(lower)alkyl which may have one or more suitable substituent(s) (e.g. benzyl, 1-naphthylmethyl, 4-methylbenzyl, 2-chlorobenzyl, 3-chlorobenzyl, 4-chlorobenzyl, etc), heterocyclic(lower)alkyl (e.g. 2-pyridylmethyl, etc) which may have one or more suitable substituent(s), or the like;

(13) heterocyclicsulfonyl;

(14) amidated carboxy such as carbamoyl,

N-heterocyclic-carbamoyl which may have one or more (preferably 1 to 3) suitable substituent(s) such as lower alkyl, halogen, or the like,

N-lower alkyl-N-heterocyclic-carbamoyl,

N-lower alkylcarbamoyl (e.g. N-methylcarbamoyl, N-ethylcarbamoyl, N-propylcarbamoyl, N-butylcarbamoyl, N-t-butylcarbamoyl, N-pentylcarbamoyl, N-hexylcarbamoyl, etc) which may have one or more (preferably 1 to 3) suitable substituent(s) such as heterocyclic group, hydroxy, or the like,

N-aryl(lower)alkylcarbamoyl such as N-(mono- or di- or tri-)phenyl(lower)alkylcarbamoyl (e.g. N-benzylcarbamoyl, N-phenethylcarbamoyl, N-benzhydrylcarbamoyl, N-tritylcarbamoyl, etc), or the like; or the like.

Suitable “heterocyclic” moiety in the terms used in the present specification may include saturated or unsaturated, monocyclic or polycyclic heterocyclic group such as

unsaturated 3 to 8-membered (more preferably 5 to 7-membered) heteromonocyclic group containing 1 to 4 nitrogen atom(s), for example, azepinyl (e.g. 1H-azepinyl, etc), pyrrolyl, pyrrolinyl, imidazolyl, pyrazolyl, pyridyl and its N-oxide, dihydropyridyl, pyrimidinyl, pyrazinyl, pyridazinyl, triazolyl (e.g. 4H-1,2,4-triazolyl, 1H-1,2,3-triazolyl, 2H-1,2,3-triazolyl, etc), tetrazolyl (e.g. 1H-tetrazolyl, 2H-tetrazolyl, etc), etc;

saturated 3 to 8-membered (more preferably 5 to 7-membered) heteromonocyclic group containing 1 to 4 nitrogen atom(s), for example, perhydroazepinyl (e.g. perhydro-1H-azepinyl, etc), pyrrolidinyl, imidazolidinyl, piperidyl, piperazinyl, etc;

unsaturated condensed heterocyclic group containing 1 to 4 nitrogen atom(s), for example, indolyl, isoindolyl, indolizinyl, benzimidazolyl, quinolyl, isoquinolyl, indazolyl, benzotriazolyl, quinoxalinyl, imidazopyridyl [e.g. imidazo[4,5-c]pyridyl, etc], tetrahydroimidazopyridyl [e.g. 4,5,6,7-tetrahydro[4,5-c]pyridyl, etc], etc;

saturated condensed heterocyclic group containing 1 to 4 nitrogen atom(s), for example, 7-azabicyclo[2.2.1]-heptyl, 3-azabicyclo[3.2.2]nonanyl, etc;

unsaturated 3 to 8-membered (more preferably 5 or 6-membered) heteromonocyclic group containing 1 to 2 oxygen atom(s) and 1 to 3 nitrogen atom(s), for example, oxazolyl, isoxazolyl, oxadiazolyl (e.g. 1,2,4-oxadiazolyl, 1,3,4-oxadiazolyl, 1,2,5-oxadiazolyl, etc), etc;

saturated 3 to 8-membered (more preferably 5 or 6-membered) heteromonocyclic group containing 1 to 2 oxygen atom(s) and 1 to 3 nitrogen atom(s), for example, morpholinyl, sydnonyl, etc;

unsaturated condensed heterocyclic group containing 1 to 2 oxygen atom(s) and 1 to 3 nitrogen atom(s), for example, benzoxazolyl, benzoxadiazolyl, etc;

unsaturated 3 to 8-membered (more preferably 5 or 6-membered) heteromonocyclic group containing 1 to 2 sulfur atom(s) and 1 to 3 nitrogen atom(s), for example, thiazolyl, isothiazolyl, thiadiazolyl (e.g. 1,2,3-thiadiazolyl, 1,2,4-thiadiazolyl, 1,3,4-thiadiazolyl, 1,2,5-thiadiazolyl, etc), dihydrothiazinyl, etc;

saturated 3 to 8-membered (more preferably 5 or 6-membered) heteromonocyclic group containing 1 to 2 sulfur atom(s) and 1 to 3 nitrogen atom(s), for example, thiazolidinyl, etc;

unsaturated condensed heterocyclic group containing 1 to 2 sulfur atom(s) and 1 to 3 nitrogen atom(s), for example, benzothiazolyl, benzothiadiazolyl, etc;

saturated condensed heterocyclic group containing 1 to 4 nitrogen atom(s), and

saturated 3 to 8-membered heteromonocyclic group containing 1 to 2 oxygen atom(s) and 1 to 3 nitrogen atom(s),

unsaturated 3 to 8-membered (more preferably 5 or 6-membered) heteromonocyclic group containing 1 to 2 oxygen atom(s), for example, furyl, etc;

unsaturated condensed heterocyclic group containing 1 to 2 oxygen atom(s), for example, benzofuranyl (e.g. benzo[b]furanyl, etc), etc;

unsaturated 3 to 8-membered (more preferably 5 or 6-membered) heteromonocyclic group containing an oxygen atom and 1 to 2 sulfur atom(s), for example, dihydrooxathiinyl, etc;

unsaturated condensed heterocyclic group containing 1 to 2 sulfur atom(s), for example, benzothienyl, benzodithiinyl, etc;

unsaturated condensed heterocyclic group containing an oxygen atom and 1 to 2 sulfur atom(s), for example, benzoxathiinyl, etc; or the like.

Suitable “aryl(lower)alkyl” may include mono-(or di- or tri-)phenyl(lower)alkyl (e.g. benzyl, phenethyl, 2-phenylpropyl, 2,4-diphenylbutyl, 1,3,5-triphenylpentyl, 6-phenylhexyl, etc), mono-(or di- or tri-)naphthyl(lower)alkyl (e.g. 2-naphthylmethyl, 2-(1-naphthyl)ethyl, 2-(2-naphthyl)ethyl, etc), and the like.

This “aryl(lower)alkyl” may have one or more (preferably 1 to 3) suitable substituent(s) selected from the group consisting of lower alkyl (e.g. methyl, ethyl, butyl, etc), higher alkyl (e.g. pentyl, etc), lower alkoxy (e.g. methoxy, ethoxy, propoxy, butoxy, t-butoxy, pentyloxy, hexyloxy, etc), aryl (e.g. phenyl, naphthyl, etc), halogen (e.g. fluoro, chloro, bromo, iodo), and the like.

Suitable “aryl(higher)alkyl” may include mono-(or di- or tri-)phenyl(higher)alkyl (e.g. 7-phenylheptyl, 6-phenylheptyl, 4,6-diphenylheptyl, 3,5,7-triphenylheptyl, 8-phenyloctyl, etc), mono-(or di- or tri-)naphthyl(higher)alkyl (e.g. 7-(2-naphthyl)heptyl, 8-(1-naphthyl)octyl, etc), and the like.

Each of the “aryl(higher)alkyl” and “heterocyclic(lower)alkyl” may have one or more (preferably 1 to 3) suitable substituent(s) as exemplified for those of “aryl(lower)alkyl” above.

Suitable “higher alkoxy” moiety in the terms used in the present specification may include straight or branched one such as heptyloxy, 2-methylheptyloxy, octyloxy, nonyloxy, decyloxy, undecyloxy, dodecyloxy, tridecyloxy, 11-methyldodecyloxy, 12-methyltridecyloxy, tetradecyloxy, pentadecyloxy, hexadecyloxy, heptadecyloxy, octadecyloxy, nonadecyloxy, icosyloxy or the like, in which the preferred one may be (C₇-C₁₆)alkoxy and the more preferred one may be nonyloxy, or decyloxy.

Suitable “cyclo(lower)alkyl” moiety in the terms used in the present specification may include the ones having 3 to 6 carbon atoms such as cyclopropyl, cyclobutyl, cyclopentyl, or cyclohexyl.

In aforesaid “acyl group”, the preferred one may be

(1) lower alkoxycarbonyl, in which the more preferred one may be (C₁-C₄)alkoxycarbonyl, and the most preferred one may be t-butoxycarbonyl;

(2) aryl(lower)alkanoyl which may have one or more suitable substituent(s), in which the more preferred one may be phenyl(lower)alkanoyl or naphthyl(lower)alkanoyl, each of which may have 1 to 3 suitable substituent(s) selected from the group consisting of carboxy(lower)alkyl (e.g. carboxymethyl, 2-carboxyethyl, 1-carboxypropyl, 4-carboxybutyl, 3-carboxypentyl, 6-carboxyhexyl, etc), protected carboxy(lower)alkyl (e.g. methoxycarbonylmethyl, 2-methoxycarbonylethyl, 2-(t-butoxycarbonyl)ethyl, etc) which may be substituted by aryl (e.g. phenyl, naphthyl, etc), protected carboxy(lower)alkenyl (e.g. 2-methoxycarbonylvinyl, etc), amidated carboxy(lower)alkyl (e.g. 2-carbamoylethyl, etc), and aryl(lower)alkyl (e.g. benzyl, phenethyl, etc), the much more preferred one may be phenyl(C₁-C₄)alkanoyl which may have 1 to 3 suitable substituent(s) selected from the group consisting of carboxymethyl, 2-carboxyethyl, methoxycarbonylmethyl, benzyloxycarbonylmethyl, 2-methoxycarbonylethyl, 2-(t-butoxycarbonyl)ethyl, 2-methoxycarbonylvinyl, 2-carbamoylethyl, benzyl, and phenethyl, or naphthyl(C₁-C₄)alkanoyl which may have benzyl, the most preferred one may be benzoyl, 2-(carboxymethyl)benzoyl, 2-(2-carboxyethyl)benzoyl, 2-(methoxycarbonylmethyl)benzoyl, 2-(benzyloxycarbonylmethyl)benzoyl, 2-(2-methoxycarbonylethyl)benzoyl, 2-[2-(t-butoxycarbonyl)ethyl]benzoyl, 2-(2-methoxycarbonylvinyl)benzoyl, 2-(2-carbamoylethyl)benzoyl, 2-benzylbenzoyl, 3-benzylbenzoyl, 2-phenethylbenzoyl, 2-naphthoyl, or 3-benzylnaphthalen-2-ylcarbonyl; or

(3) heterocyclic(lower)alkanoyl which may have one or more suitable substituent(s), in which the more preferred one may be heterocyclic(lower)alkanoyl, wherein heterocyclic moiety is unsaturated condensed heterocyclic group containing 1 to 4 nitrogen atom(s), which may have 1 to 3 lower alkylaryl(lower)alkyl, haloaryl(lower)alkyl, or heterocyclic(lower)alkyl, wherein heterocyclic moiety is unsaturated 3 to 8-membered heteromonocyclic group containing 1 to 4 nitrogen atom(s), the more preferred one may be quinolyl(C₁-C₄)alkanoyl, isoquinolyl(C₁-C₄)alkanoyl, or indolyl(C₁-C₄)alkanoyl which may have (C₁-C₄)alkylphenyl(C₁-C₄)alkyl, halophenyl(C₁-C₄)alkyl, or pyridyl(C₁-C₄)alkyl, the much more preferred one may be quinolylcarbonyl, isoquinolylcarbonyl, or indolylcarbonyl which may have benzyl, 1-naphthylmethyl, 4-methylbenzyl, 2-chlorobenzyl, 3-chlorobenzyl, 4-chlorobenzyl or 2-pyridylmethyl, the most preferred one may be 2-(or 3-)quinolylcarbonyl, 1-(or 3-)isoquinolylcarbonyl, 1-benzylindol-2-(or 3-)ylcarbonyl, 1-(1-naphthylmethyl)indol-3-ylcarbonyl, 1-(4-methylbenzyl)indol-2-(or 3-)ylcarbonyl, 1-[2-(or 3- or 4-)chlorobenzyl]indol-3-ylcarbonyl or 1-(2-pyridylmethyl)indol-3-ylcarbonyl.

The preferred “acyl” moiety in the term “acyl(lower)alkyl” may be carboxy; protected carboxy, in which the more preferred one may be lower alkoxycarbonyl or aryl(lower)alkoxycarbonyl, the much more preferred one may be (C₁-C₄)alkoxycarbonyl or phenyl(C₁-C₄)alkoxycarbonyl, and the most preferred one may be methoxycarbonyl or benzyloxycarbonyl; or amidated carboxy, in which the more preferred one may be carbamoyl.

The preferred “substituent” in the terms “aryl(lower)alkyl which may have one or more suitable substituent(s)”, “aryl(higher)alkyl which may have one or more suitable substituent(s)” and “heterocyclic(lower)alkyl which may have one or more suitable substituent(s)” may include lower alkyl as exemplified above, preferably (C₁-C₄)alkyl, more preferably methyl, ethyl or butyl; higher alkyl as exemplified above, preferably (C₇-C₁₆)alkyl, more preferably heptyl;

aryl as exemplified above, preferably phenyl; or the like.

In the following, some of the preferred embodiments of the fatty acid derivative (I) of the present invention are shown.

(1) the derivative (I), wherein

R¹ is protected carboxy;

aryl(lower)alkanoyl which may have 1 to 3 suitable substituent(s) selected from the group consisting of lower alkoxy, aryl, carboxy(lower)alkyl, protected carboxy(lower)alkyl which may be substituted by aryl, protected carboxy(lower)alkenyl, amidated carboxy(lower)alkyl, and aryl(lower)alkyl which may have 1 to 3 suitable substituent(s) selected from the group consisting of lower alkyl, higher alkyl, lower alkoxy, aryl and halogen;

heterocyclic(lower)alkanoyl which may have 1 to 3 suitable substituent(s) selected from the group consisting of lower alkyl, aryl(lower)alkyl which may have 1 to 3 suitable substituent(s) selected from the group consisting of lower alkyl, higher alkyl, lower alkoxy, aryl and halogen, and heterocyclic(lower)alkyl which may have 1 to 3 suitable substituent(s) selected from the group consisting of lower alkyl, higher alkyl, lower alkoxy, aryl and halogen;

R² is carboxy(lower)alkyl or protected carboxy(lower)alkyl,

R³ is hydrogen;

aryl(lower)alkyl which may have 1 to 3 suitable substituent(s) selected from the group consisting of lower alkyl, higher alkyl, lower alkoxy, aryl and halogen;

aryl(higher)alkyl which may have 1 to 3 suitable substituent(s) selected from the group consisting of lower alkyl, higher alkyl, lower alkoxy, aryl and halogen;

heterocyclic(lower)alkyl which may have 1 to 3 suitable substituent(s) selected from the group consisting of lower alkyl, higher alkyl, lower alkoxy, aryl and halogen;

higher alkoxy(lower)alkyl;

lower alkyl; or

higher alkyl,

R⁴ is carbamoyl(lower)alkyl, and

X is —O—, —NH— or

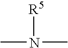

[wherein R⁵ is lower alkyl, [cyclo(lower)alkyl]-(lower)alkyl, aryl(lower)alkyl, or heterocyclic(lower)alkyl],

with proviso that X is

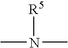

(wherein R⁵ is as defined above), when R³ is lower alkyl or higher alkyl.

(2) the derivative (I), wherein

R¹ is esterified carboxy (preferably lower alkoxycarbonyl);

phenyl(lower)alkanoyl or naphthyl(lower)alkanoyl, each of which may have 1 to 3 suitable substituent(s) selected from the group consisting of carboxy(lower)alkyl, esterified carboxy(lower)alkyl (preferably lower alkoxycarbonyl(lower)alkyl) which may be substituted by phenyl, esterified carboxy(lower)alkenyl (preferably lower alkoxycarbonyl(lower)alkenyl), carbamoyl(lower)alkyl and phenyl(lower)alkyl; or heterocyclic(lower)alkanoyl which may have 1 to 3 suitable substituent(s) selected from the group consisting of pyridyl(lower)alkyl, naphthyl(lower)alkyl and phenyl(lower)alkyl which may have 1 to 3 suitable substituent(s) selected from the group consisting of lower alkyl and halogen, in which the heterocyclic moiety is unsaturated condensed heterocyclic group containing 1 to 4 nitrogen atom(s),

R² is carboxy(lower)alkyl or esterified carboxy(lower)alkyl (preferably methoxycarbonyl(lower)alkyl or benzyloxycarbonyl(lower)alkyl),

R³ is hydrogen;

phenyl(lower)alkyl which may have 1 to 3 suitable substituent(s) selected from the group consisting of lower alkyl, higher alkyl and phenyl; or naphthyl(lower)alkyl which may be substituted by lower alkyl,

R⁴ is carbamoyl(lower)alkyl, and

X is —O—.

(3) the derivative (I), wherein

R¹, R², R³ and R⁴ are each as defined above in (2), and

X is —NH— or

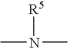

[wherein R⁵ is lower alkyl, phenyl(lower)alkyl, or pyridyl(lower)alkyl].

(4) the derivative (I), wherein

R¹, R², R⁴ and X are each as defined above in (2), and R³ is phenyl(higher)alkyl.

(5) the derivative (I), wherein

R¹, R², R³ and R⁴ are each as defined above in (4), and

X is —NH— or

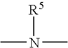

[wherein R⁵ is as defined above in (3)].

(6) the derivative (I), wherein

R¹, R², R⁴ and X are each as defined above in (2), and

R³ is heterocyclic(lower)alkyl, in which the heterocyclic moiety is unsaturated condensed heterocyclic group containing 1 to 2 oxygen atom(s).

(7) the derivative (I), wherein

R¹, R², R³ and R⁴ are each as defined above in (6), and

X is —NH— or

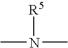

[wherein R⁵ is as defined above in (3)].

(8) the derivative (I), wherein

R¹, R², R⁴ and X are each as defined above in (2), and

R³ is higher alkoxy(lower)alkyl.

(9) the derivative (I), wherein

R¹, R², R³ and R⁴ are each as defined above in (8), and

X is —NH— or

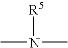

[wherein R⁵ is as defined above in (3)].

(10) the derivative (I), wherein

R¹, R² and R⁴ are each as defined above in (2), and

R³ is lower alkyl, and

X is

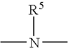

[wherein R⁵ is as defined above in (3)].

(11) the derivative (I), wherein

R¹, R² and R⁴ are each as defined above in (2), and

R³ is higher alkyl, and

X is

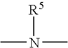

[wherein R⁵ is as defined above in (3)].

(12) the derivative (I), wherein

R¹ is (C₁-C₄)alkoxycarbonyl;

phenyl(C₁-C₄)alkanoyl or naphthyl(C₁-C₄)alkanoyl, each of which may have carboxy(C₁-C₄)alkyl, (C₁-C₄)alkoxycarbonyl(C₁-C₄)alkyl which may be substituted by phenyl, (C₁-C₄)alkoxycarbonyl-(C₂-C₄)alkenyl, carbamoyl(C₁-C₄)alkyl or phenyl(C₁-C₄)alkyl;

heterocyclic(C₁-C₄)alkanoyl which may have pyridyl(C₁-C₄)alkyl, naphthyl(C₁-C₄)alkyl or phenyl(C₁-C₄)alkyl which may have 1 to 3 suitable substituent(s) selected from the group consisting of (C₁-C₄)alkyl and chloro, in which the heterocyclic moiety is indolyl, quinolyl or isoquinolyl,

R² is carboxy(C₁-C₄)alkyl, methoxycarbonyl(C₁-C₄)alkyl, or benzyloxy(C₁-C₄)alkyl,

R³ is hydrogen;

phenyl(C₁-C₄)alkyl which may have (C₁-C₄)alkyl, (C₇-C₁₆)alkyl or phenyl; or

naphthyl(C₁-C₄)alkyl which may have (C₁-C₄)alkyl,

R⁴ is carbamoyl(C₁-C₄)alkyl, and

X is —O—.

(13) the derivative (I), wherein

R¹, R², R³ and R⁴ are each as defined above in (12), and

X is —NH— or

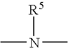

[wherein R⁵ is (C₁-C₅)alkyl, phenyl(C₁-C₄)alkyl, or pyridyl(C₁-C₄)alkyl].

(14) the derivative (I), wherein

R¹, R², R⁴ and X are each as defined above in (12), and

R³ is phenyl(C₇-C₁₆)alkyl.

(15) the derivative (I), wherein

R¹, R², R³ and R⁴ are each as defined above in (14), and

X is —NH— or

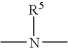

[wherein R⁵ is as defined above in (13)].

(16) the derivative (I), wherein

R¹, R², R⁴ and X are each as defined above in (12), and

R³ is benzofuranyl(C₁-C₄)alkyl.

(17) the derivative (I), wherein

R¹, R², R³ and R⁴ are each as defined above in (16), and

X is —NH— or

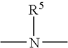

[wherein R⁵ is as defined above in (13)].

(18) the derivative (I), wherein

R¹, R², R⁴ and X are each as defined above in (12), and

R³ is (C₇-C₁₆)alkoxy(C₁-C₄)alkyl.

(19) the derivative (I), wherein

R¹, R², R³ and R⁴ are each as defined above in (18), and

X is —NH— or

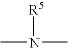

[wherein R⁵ is as defined above in (13)].

(20) the derivative (I), wherein

R¹, R², R⁴ and X are each as defined above in (12), and

R³ is (C₃-C₆)alkyl.

(21) the derivative (I), wherein

R¹, R², R³ and R⁴ are each as defined above in (20), and

X is

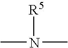

[wherein R⁵ is as defined above in (13)].

(22) the derivative (I), wherein

R¹, R², R⁴ and X are each as defined above in (12), and

R³ is (C₇-C₁₆)alkyl.

(23) the derivative (I), wherein

R¹, R², R³ and R⁴ are each as defined above in (22), and

X is

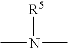

[wherein R⁵ is as defined above in (15)].

The processes for preparing the object compound (I) of the present invention are explained in detail in the following.

Process 1

The compound (I) or a salt thereof can be prepared by reacting the compound (II) or a reactive derivative at the carboxy group or a salt thereof with the compound (III) or a salt thereof.

Suitable salts of the compounds (II) and (III) can be referred to the ones as exemplified for the compound (I).

Suitable reactive derivative at the carboxy group of the compound (II) may include an acid halide, an acid anhydride, an activated amide, an activated ester, and the like. Suitable examples of the reactive derivatives may be an acid chloride; an acid azide; a mixed acid anhydride with an acid such as substituted phosphoric acid [e.g. dialkylphosphoric acid, phenylphosphoric acid, diphenylphosphoric acid, dibenzylphosphoric acid, halogenated phosphoric acid, etc], dialkylphosphorous acid, sulfurous acid, thiosulfuric acid, sulfuric acid, sulfonic acid [e.g. methanesulfonic acid, etc], aliphatic carboxylic acid [e.g. acetic acid, propionic acid, butyric acid, isobutyric acid, pivalic acid, pentanoic acid, isopentanoic acid, 2-ethylbutyric acid, trichloroacetic acid, etc] or aromatic carboxylic acid [e.g. benzoic acid, etc]; a symmetrical acid anhydride; an activated amide with imidazole, 4-substituted imidazole, dimethylpyrazole, triazole, tetrazole or 1-hydroxy-1H-benzotriazole; or an activated ester [e.g. cyanomethyl ester, methoxymethyl ester, dimethyliminomethyl [(CH₃)₂ [symbol] ═CH—]ester, vinyl ester, propargyl ester, p-nitrophenyl ester, 2,4-dinitrophenyl ester, trichlorophenyl ester, pentachlorophenyl ester, mesylphenyl ester, phenylazophenyl ester, phenyl thioester, p-nitrophenyl thioester, p-cresyl thioester, carboxymethyl thioester, pyranyl ester, pyridyl ester, piperidyl ester, 8-quinolyl thioester, etc] or an ester with a N-hydroxy compound [e.g. N,N-dimethylhydroxylamine, 1-hydroxy-2-(1H)-pyridone, N-hydroxysuccinimide, N-hydroxyphthalimide, 1-hydroxy-1H-benzotriazole, etc], and the like. These reactive derivatives can optionally be selected from them according to the kind of the compound (II) to be used.

In the case that the group X is

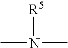

in the compound (III), the compound (III) can be used in the form of its reactive derivative at the amino group.

Suitable said reactive derivative at the amino group may include Schiff's base type imino or its tautomeric enamine type isomer formed by the reaction of the compound (III) with a carbonyl compound such as aldehyde, ketone or the like; a silyl derivative formed by the reaction of the compound (III) with a silyl compound such as bis(trimethylsilyl)acetamide, mono(trimethylsilyl)acetamide, bis(trimethylsilyl)urea or the like; a derivative formed by reaction of the compound (III) with phosphorus trichloride or phosgene, and the like.

The reaction is usually carried out in a conventional solvent such as water, alcohol [e.g. methanol, ethanol, etc], acetone, dioxane, acetonitrile, chloroform, methylene chloride, ethylene chloride, tetrahydrofuran, ethyl acetate, N,N-dimethylformamide, pyridine or any other organic solvent which does not adversely influence the reaction. These conventional solvents may also be used in a mixture with water.

In this reaction, when the compound (II) is used in a free acid form or its salt form, the reaction is preferably carried out in the presence of a conventional condensing agent such as N,N′-dicyclohexylcarbodiimide; 1-ethyl-3-(3-dimethylaminopropyl)carbodiimide; benzotriazole-1-yloxy-tris-pyrrolidinophosphonium hexafluorophosphate, or the like.

The reaction may also be carried out in the presence of an inorganic or organic base such as an alkali metal carbonate, alkali metal bicarbonate, tri(lower)alkylamine (e.g. triethylamine, etc), pyridine, di(lower)alkylamino-pyridine (e.g. N,N-dimethylaminopyridine, etc), N-(lower)-alkylmorpholine, N,N-di(lower)alkylbenzylamine, or the like.

The reaction temperature is not critical, and the reaction is usually carried out under cooling to warming.

Process 2

The compound (I) or a salt thereof can be prepared by reacting the compound (IV) or a reactive derivative at the amino group or a salt thereof with the compound (V) or a reactive derivative or a salt thereof.

Suitable salts of the compounds (IV) and (V) can be referred to the ones as exemplified for the compound (I).

The reaction of this process can be carried out according to a similar manner to that of Process 1, and so the reaction condition can be referred to the explanation therein.

Process 3

The compound (Ib) or a salt thereof can be prepared by subjecting a compound (Ia) or a salt thereof to elimination reaction of carboxy protective group.

This reaction is carried out in accordance with a conventional method such as hydrolysis, reduction or the like.

The hydrolysis is preferably carried out in the presence of a base or an acid including Lewis acid.

Suitable base may include an inorganic base and an organic base such as an alkali metal [e.g. sodium, potassium, etc], an alkaline earth metal [e.g. magnesium, calcium, etc], the hydroxide or carbonate or bicarbonate thereof, trialkylamine [e.g. trimethylamine, triethylamine, etc], picoline, 1,5-diazabicyclo[4.3.0]non-5-ene, 1,4-diazabicyclo[2.2.2]octane, 1,8-diazabicyclo[5.4.0]undec-7-ene, or the like.

Suitable acid may include an organic acid [e.g. formic acid, acetic acid, propionic acid, trichloroacetic acid, trifluoroacetic acid, etc] and an inorganic acid [e.g. hydrochloric acid, hydrobromic acid, sulfuric acid, hydrogen chloride, hydrogen bromide, etc].

The elimination using Lewis acid such as trihaloacetic acid [e.g. trichloroacetic acid, trifluoroacetic acid, etc], aluminium halide [e.g. aluminium chloride, etc] or the like is preferably carried out in the presence of cation trapping agents [e.g. anisole, phenol, etc].

The reaction is usually carried out in a solvent such as water, an alcohol [e.g. methanol, ethanol, etc], nitromethane, methylene chloride, tetrahydrofuran, a mixture thereof or any other solvent which does not adversely influence the reaction. A liquid base or acid can be also used as the solvent. The reaction temperature is not critical and the reaction is usually carried out under cooling to warming.

The reduction method applicable for the elimination reaction may include chemical reduction and catalytic reduction.

Suitable reducing agents to be used in chemical reduction are a combination of metal [e.g. tin, zinc, iron, etc] or metallic compound [e.g. chromium chloride, chromium acetate, etc] and an organic or inorganic acid [e.g. formic acid, acetic acid, propionic acid, trifluoroacetic acid, p-toluenesulfonic acid, hydrochloric acid, hydrobromic acid, etc].

Suitable catalysts to be used in catalytic reduction are conventional ones such as platinum catalysts [e.g. platinum plate, spongy platinum, platinum black, colloidal platinum, platinum oxide, platinum wire, etc], palladium catalysts [e.g. spongy palladium, palladium black, palladium oxide, palladium on carbon, colloidal palladium, palladium on barium sulfate, palladium on barium carbonate, etc], nickel catalysts [e.g. reduced nickel, nickel oxide, Raney nickel, etc], cobalt catalysts [e.g. reduced cobalt, Raney cobalt, etc], iron catalysts [e.g. reduced iron, Raney iron, etc], copper catalysts [e.g. reduced copper, Raney copper, Ullman copper, etc] and the like.

The reduction is usually carried out in a conventional solvent which does not adversely influence the reaction such as water, methanol, ethanol, propanol, dioxane, N,N-dimethylformamide, or a mixture thereof. Additionally, in case that the above-mentioned acids to be used in chemical reduction are in liquid, they can also be used as a solvent. Further, a suitable solvent to be used in catalytic reduction may be the above-mentioned solvent, and other conventional solvent such as diethyl ether, dioxane, tetrahydrofuran, etc, or a mixture thereof.

The reaction temperature of this reduction is not critical and the reaction is usually carried out under cooling to warming.

It is to be noted the compound (I) or a salt thereof can be prepared by the methods other than aforesaid Processes 1 to 3, for examples, by the other methods disclosed in Examples in this specification.

Biological Property of the Compound (I)

In order to show the utility of the object compound (I), the biological test data on phospholipase A₂ assay of the representative compound of the compound (I) is shown in the following.

Test on the Inhibitory Effect Against Phospholipase A₂ (PLA₂)

[I] Test Method

PLA₂ activity was assayed with a fluorescent phospholipid analogue [1-palmitoyl-2-(10-pyrenyldecanoyl)-sn-glycero-3-monomethylphosphatidic acid (10-pyrene PA-monomethyl ester)] as a substrate, according to Radvanyi et al. (1989) with several modifications. Briefly, the reaction medium was prepared by sequential additon of 1160 μl of 50 mM Tris-HCl (pH 7.4) buffer containing 100 mM NaCl and 1 mM EDTA, 10 μl of 120 μM 10-pyrene PA-monomethyl ester in ethanol, 10 μl of drug sample in methanol and 10 μl of 1.2 μg/ml human recombinant PLA₂ group II enzyme. The enzymatic reaction was then initiated with 10 μl of 0.84 M CaCl₂. Following incubation at room temperature for 10 minutes, the reaction was terminated by addition of 25 μl of 1 M EDTA and 10 μl of 10 mg/ml β-cyclodextrin. Fluorescence measurements were carried out with a JASCO Corporation FP-777 spectrofluorometer. Excitation and emission wavelengths were 345 nm and 380 nm, respectively. All data are the average of at least duplicate determinations corrected for the spontaneous fluorescence of the reaction medium. Data were expressed as percent inhibition.

### REFERENCE

F. Radvanyi, L. Jordan, F. Russo-Marie and C. Bon: A sensitive and continuous fluorometric assay for phospholipase A₂ using pyrene-labeled phospholipids in the presence of serum albumin. [Anal. Biochem. 177 pages 103-109 (1989)]

[II] Test Compound

(3S)-3-[(2S)-2-(3-Benzylnaphthalen-2-ylcarbonylamino)-5-carboxypentanoyl]oxy-4-(2-naphthyl)butanamide (the compound of Example 24)

[III] Test Result

| Percent inhibition | |
|---|---|
| dose (M) | inhibition (%) |
| 1 × 10⁻⁶ | 100 |

The pharmaceutical composition of the present invention can be used in the form of a pharmaceutical preparation, for example, in solid, semisolid or liquid form, which contains the object compound (I) or a pharmaceutically acceptable salt thereof, as an active ingredient in admixture with an organic or inorganic carrier or excipient suitable for rectal, pulmonary (nasal or buccal inhalation), nasal, ocular, external (topical), oral or parenteral (including subcutaneous, intravenous, intramuscular and intra-articular) administrations or insufflation.

The active ingredient may be compounded, for example, with the usual non-toxic, pharmaceutically acceptable carriers for tablets, pellets, troches, capsules, suppositories, creams, ointments, aerosols, powders for insufflation, solutions, emulsions, suspensions, and any other form suitable for use. And, if necessary, in addition, auxiliary, stabilizing, thickening and coloring agents and perfumes may be used.

The object compound (I) or a pharmaceutically acceptable salt thereof is/are included in the pharmaceutical composition in an amount sufficient to produce the desired effect upon the process or condition of the diseases.

The pharmaceutical composition of the present invention can be manufactured by the conventional method in this field of the art. If necessary, the technique generally used in this field of the art for improving the bioavailability of a drug can be applied to the pharmaceutical composition of the present invention.

For applying the composition to a human being or an animal, it is preferable to apply it by intravenous (including i.v. infusion), intramuscular, pulmonary, or oral administration, or insufflation including aerosols from metered dose inhalator, nebulizer or dry powder inhalator.

While the dosage of therapeutically effective amount of the object compound (I) varies from and also depends upon the age and condition of each individual patient to be treated, in the case of intravenous administration, a daily dose of 0.001-100 mg of the object compound (I) per kg weight of a human being or an animal, in the case of intramuscular administration, a daily dose of 0.001-100 mg of the object compound (I) per kg weight of a human being or an animal, in the case of oral administration, a daily dose of 0.001-200 mg of the object compound (I) per kg weight of a human being or an animal is generally given for the prevention and/or the treatment of aforesaid diseases in a human being or an animal.

The following preparations and examples are given only for the purpose of illustrating the present invention in more detail.

Preparation 1

2-(6-Ethylnaphthalen-2-yl)acetic acid (1.88 g) was dissolved in thionyl chloride (9.6 ml) and the mixture was stirred at room temperature for 1 hour and then the mixture was concentrated in vacuo. The residue was dissolved in methylene chloride (20 ml) and the solution was added dropwise to a stirring solution of Meldrum's acid (1.26 g) and pyridine (1.56 ml) in methylene chloride (20 ml) at room temperature. After being stirred overnight at the same temperature, the mixture was washed with 1N hydrochloric acid and the organic layer was dried over magnesium sulfate and concentrated in vacuo. The residue was dissolved in methanol (40 ml) and refluxed for 2 hours and the mixture was concentrated in vacuo. The residue was dissolved in ethyl acetate and the solution was washed with 1N hydrochloric acid, water, aqueous sodium bicarbonate and brine, successively. The organic layer was dried over magnesium sulfate and concentrated in vacuo. The residue was purified by silica gel column chromatography (ethyl acetate:hexane=1:9, as an eluent) to give methyl 4-(6-ethyl-2-naphthyl)-3-oxobutanoate (0.61 g).

NMR (CDCl₃, δ): 7.74 (2H, dd, J=8.0, 7.5 Hz), 7.64 (1H, s), 7.60 (1H, s), 7.36 (1H, d, J=8.0 Hz), 7.28 (1H, d, J=8.0 Hz), 3.96 (2H, s), 3.70 (3H, s), 3.46 (2H, s), 2.80 (2H, q, J=7.5 Hz), 1.30 (3H, t, J=7.5 Hz)

ESI-MS: 271 [M+H]

Preparation 2

Methyl 4-(2-naphthyl)-3-oxobutanoate was obtained according to a similar manner to that of Preparation 1.

NMR (CDCl₃, δ): 7.82 (3H, m), 7.70 (1H, s), 7.48 (2H, m), 7.32 (1H, m), 4.00 (2H, s), 3.70 (3H, s), 3.48 (2H, s)

Preparation 3

Methyl 4-(6-ethyl-2-naphthyl)-3-oxobutanoate (0.60 g), D-camphorsulfonic acid (4.1 mg) and [Ru2Cl2((S)-BINAP)₂]NEt3 (di[(S)-2,2′-bis(diphenylphosphino)-1,1′-binaphthyl]-dichlorodirhutenium triethylamine complex) (4.5 mg) in methanol (6 ml) was hydrogenated at 65° C. under hydrogen atmosphere under 10 atmosphere for 5 hours. After cooling at room temperature, the solvent was removed in vacuo and the residue was purified by silica gel column chromatography (ethyl acetate:hexane=1:4, as an eluent) to give methyl (3S)-4-(6-ethyl-2-naphthyl)-3-hydroxybutanoate (0.55 g).

NMR (CDCl₃, δ): 7.72 (2H, dd, J=8.0, 6.0 Hz), 7.64 (1H, s), 7.60 (1H, s), 7.32 (2H, t, J=7.5 Hz), 4.36 (1H, br s), 3.70 (3H, s), 2.96 (2H, ABX), 2.80 (2H, a, J=7.5 Hz), 2.52 (2H, ABX), 1.30 (3H, t, J=7.5 Hz)

ESI-MS: 273 [M+H]

The following compounds (Preparations 4 and 5) were obtained according to a similar manner to that of Preparation 3.

Preparation 4

Methyl (3R)-3-hydroxy-4-(2-naphthyl)butanoate

NMR (CDCl₃, δ): 7.74-7.84 (3H, m), 7.66 (1H, s), 7.4-7.5 (2H, m), 7.34 (1H, d, J=8.0 Hz), 4.36 (1H, m), 3.70 (3H, s), 2.98 (2H, ABX), 2.87 (1H, d, J=5.0 Hz), 2.52 (2H, ABX)

ESI-MS: 245 [M+H]

Preparation 5

Methyl (3S)-3-hydroxy-4-(2-naphthyl)butanoate

NMR (CDCl₃, δ): 7.74-7.84 (3H, m), 7.66 (1H, s), 7.4-7.5 (2H, m), 7.34 (1H, d, J=8.0 Hz), 4.36 (1H, m), 3.70 (3H, s), 2.98 (2H, ABX), 2.87 (1H, d, J=5.0 Hz), 2.52 (2H, ABX)

ESI-MS: 245 [M+H]

Preparation 6

To a solution of methyl (3R)-3-hydroxy-4-(2-naphthyl)butanoate (3.02 g) and methanesulfonyl chloride (2.12 g) in methylene chloride (60 ml) was added triethylamine (2.5 g) at 0° C. After being stirred at room temperature for 1.5 hours, the mixture was diluted with ethyl ether and the mixture was washed with 0.5N hydrochloric acid, water, aqueous sodium bicarbonate and brine, successively. The organic layer was dried over magnesium sulfate and concentrated in vacuo. The residue was dissolved in DMF (50 ml) and sodium azide (2.02 g) was added to this solution. After being stirred at 60° C. for 1 hour, the mixture was diluted with ethyl ether and the mixture was washed with water and brine. The organic layer was dried with magnesium sulfate and concentrated in vacuo. The residue was dissolved in methanol (100 ml) and hydrogenated at room temperature under hydrogen atmosphere under atmospheric pressure for 6 hours. The catalyst was filtered off and the solvent was removed under reduced pressure. The residue was dissolved in 4N hydrogen chloride in ethyl acetate (50 ml) at room temperature. After being stirred at the same temperature for 10 minutes, the mixture was concentrated in vacuo and the residue was triturated with ethyl ether to give methyl (3S)-3-amino-4-(2-naphthyl)butanoate hydrochloride (0.44 g).

NMR (CDCl₃—CD₃OD, δ): 7.74-7.84 (3H, m), 7.72 (1H, s), 7.42-7.52 (2H, m), 7.34 (1H, d, J=8.0 Hz), 3.90 (1H, m), 3.46 (1H, dd, J=15.0, 5.0 Hz), 3.12 (1H, dd, J=15.0, 10.0 Hz), 2.80 (2H, ABX)

Preparation 7

Methyl (3S)-4-(6-ethyl-2-naphthyl)-3-hydroxybutanoate (0.54 g) was dissolved in 15N ammonia in methanol (5 ml) at room temperature and the mixture was allowed to stand for six days. The solvent was evaporated in vacuo and the residue was triturated with isopropyl ether to give (3S)-4-(6-ethyl-2-naphthyl)-3-hydroxybutanamide (0.49 g).

NMR (DMSO-d₆, δ): 7.76 (2H, dd, J=8.0, 4.0 Hz), 7.64 (2H, s), 7.34 (2H, d, J=8.0 Hz), 7.28 (1H, br s), 6.80 (1H, br s), 4.86 (1H, d, J=4.0 Hz), 4.14 (1H, m), 2.80 (2H, d, J=7.5 Hz), 2.74 (2H, q, J=7.5 Hz), 2.16 (2H, d, J=7.5 Hz), 1.26 (3H, t, J=7.5 Hz)

The following compounds (Preparations 8 and 9) were obtained according to a similar manner to that of Preparation 7.

Preparation 8

(3S)-3-Amino-4-(2-naphthyl)butanamide

NMR (CDCl₃, δ): 7.8 (4H, m), 7.64 (1H, s), 7.44 (3H, m), 7.30 (1H, d, J=10.0 Hz), 3.5 (1H, m), 3.00 (1H, dd, J=12.0, 7.5 Hz), 2.76 (1H, dd, J=12.0, 10.0 Hz), 2.48 (1H, dd, J=15.0, 5.0 Hz), 2.25 (1H, dd, J=15.0, 10.0 Hz)

ESI-MS: 229 [M+H]

Preparation 9

(3S)-3-Hydroxy-4-(2-naphthyl)butanamide

NMR (DMSO-d₆, δ): 7.78-7.90 (3H, m), 7.70 (1H, s), 7.34-7.52 (3H, m), 7.30 (1H, br s), 6.82 (1H, br s), 4.90 (1H, d, J=5.0 Hz), 4.14 (1H, m), 2.74-2.90 (2H, m), 2.15 (2H, d, J=5.0 Hz)

Preparation 10

A mixture of 2-acetyl-6-ethylnaphthalene (9.09 g) and morpholine (6 ml) and sulfur (2.2 g) was heated at 120° C. for one hour and then refluxed for ten hours. The mixture was cooled to room temperature and diluted with ethyl acetate. The mixture was washed with 1N hydrochloric acid, aqueous sodium bicarbonate and brine, successively. The organic layer was dried over magnesium sulfate and concentrated in vacuo. The residue was purified by silica gel column chromatography (ethyl acetate:hexane=1:1, as an eluent) to give 6-ethyl-2-naphthylacetothiomorpholide. The thiomorpholide thus obtained was dissolved in acetic acid (20 ml), concentrated sulfuric acid (3 ml) and water (4.5 ml) and the mixture was refluxed for five hours. The mixture was cooled to room temperature and poured into ethyl acetate and the mixture was washed with water and brine, successively. The organic layer was dried over magnesium sulfate and concentrated in vacuo. The residue was triturated with isopropyl ether to give 2-(6-ethylnaphthalen-2-yl)acetic acid (1.90 g).

NMR (CDCl₃, δ): 7.74 (2H, t, J=7.5 Hz), 7.70 (1H, s), 7.60 (1H, s), 7.36 (2H, t, J=7.5 Hz), 3.80 (2H, s), 2.80 (2H, q, J=7.5 Hz), 1.30 (3H, t, J=7.5 Hz)

ESI-MS: 213 [M−H]

Preparation 11

To an ice-cooled suspension of sodium hydride (60% in oil dispersion, 6.75 g) in tetrahydrofuran (50 ml) was added 5-hydroxy-1-pentene (10.1 g) in tetrahydrofuran (50 ml). After stirring for 30 minutes, 1-bromononane (31.1 g) in tetrahydrofuran (100 ml) was added. This mixture was refluxed overnight, poured into saturated aqueous ammonium chloride (300 ml), and extracted with diethyl ether (300 ml). The organic phase was separated, washed with water (300 ml) and brine (200 ml), dried over magnesium sulfate, and evaporated to dryness. The residue was chromatographed on a silica gel (1000 cc), eluting with ethyl acetate in n-hexane (0-10%) to give 4-pentenyl nonyl ether (17.2 g).

NMR (CDCl₃, δ): 5.83 (1H, m), 4.92-5.07 (2H, m), 3.35-3.45 (4H, m), 2.12 (2H, m), 1.48-1.73 (4H, m), 1.17-1.39 (12H, m), 0.87 (3H, t, J=7 Hz)

Preparation 12

A solution of 4-pentenyl nonyl ether (7.0 g) in a mixture of methanol (150 ml) and dichloromethane (50 ml) was cooled to −78° C. Ozone was passed through this solution keeping temperature below −60° C. until the color turned to be light blue. Then, methylsulfide (12.1 ml) was added dropwise, and this solution was warmed to room temperature over 3 hours. The resulting solution was concentrated and partitioned between diethyl ether (150 ml) and water (100 ml). The ethereal solution was dried over magnesium sulfate and evaporated to dryness to give a crude product of 1,1-dimethoxy-4-nonyloxybutane (7.76 g).

NMR (CDCl₃, δ): 4.38 (1H, t, J=5 Hz), 3.42 (2H, t, J=6 Hz), 3.38 (2H, t, J=6 Hz), 3.32 (6H, s), 1.37-1.73 (6H, m), 1.17-1.41 (12H, m), 0.88 (3H, t, J=7 Hz)

Preparation 13

To an ice-cooled solution of 1,1-dimethoxy-4-nonyloxybutane (3.00 g) in acetone (150 ml) was added 2N Jones' reagent drop by drop. After stirring for 1 hour at 4° C., isopropyl alcohol was added until the orange color disappeared. This solution was neutralized with 1N aqueous sodium hydroxide, concentrated in vacuo, acidified with 1N hydrochloric acid, saturated with ammonium chloride, and extracted with ethyl acetate (50 ml). The organic phase was washed with brine, dried over magnesium sulfate, and evaporated to dryness to give 4-nonyloxybutyric acid (2.68 g).

NMR (CDCl₃, δ): 3.35-3.52 (4H, m), 2.48 (2H, t, J=7 Hz), 1.90 (2H, m), 1.47-1.66 (2H, m), 1.16-1.41 (12H, m), 0.88 (3H, t, J=7 Hz)

Preparation 14

To a solution of 4-nonyloxybutyric acid (2.66 g) and a drop of dimethylformamide in dichloromethane (50 ml) was added oxalyl chloride (1.11 ml). This solution was stirred for 1 hour and concentrated under reduced pressure. The residue was dissolved in dichloromethane (10 ml), and added to a solution of Meldrum's acid (1.66 g) and pyridine (1.87 ml) in dichloromethane (25 ml) at 4° C. This solution was stirred at room temperature overnight. The resulting mixture was washed with 10% hydrochloric acid (50 ml×3) and water, dried over magnesium sulfate, and concentrated in vacuo. The residue was dissolved in methanol, and refluxed for 3 hours. Then, the mixture was evaporated in dryness, and chromatographed on a silica gel (150 cc) eluting with 10% ethyl acetate in n-hexane to give methyl 6-nonyloxy-3-oxohexanoate (0.96 g).

NMR (CDCl₃, δ): 3.74 (3H, s), 3.47 (2H, s), 3.41 (2H, t, J=6 Hz), 3.36 (2H, t, J=6 Hz), 2.63 (2H, t, J=7 Hz), 1.86 (2H, m), 1.47-1.61 (2H, m), 1.18-1.40 (12H, m), 0.88 (3H, t, J=7 Hz)

Preparation 15

Methyl (3S)-3-hydroxy-6-nonyloxyhexanoate was obtained according to a similar manner to that of Preparation 3.

NMR (CDCl₃, δ): 4.04 (1H, m), 3.71 (3H, s), 3.49 (1H, d, J=3 Hz), 3.45 (2H, t, J=6 Hz), 3.41 (2H, t, J=7 Hz), 2.41-2.53 (2H, m), 1.46-1.80 (6H, m), 1.17-1.38 (12H, m), 0.88 (3H, t, J=7 Hz)

Preparation 16

(3S)-3-Hydroxy-6-nonyloxyhexanamide was obtained according to a similar manner to that of Preparation 7.

NMR (CDCl₃, δ): 6.37 (1H, br s), 5.33 (1H, br s), 4.39 (1H, d, J=2 Hz), 3.99 (1H, m), 3.40-3.53 (4H, m), 2.30-2.44 (2H, m), 1.49-1.82 (6H, m), 1.18-1.41 (12H, m), 0.87 (3H, t, J=7 Hz)

Preparation 17

To an ice-cooled solution of methyl (3R)-3-hydroxyhexadecanoate (5.35 g) and triethylamine (5.21 ml) in dichloromethane (50 ml) was added methanesulfonyl chloride (2.17 ml). After stirring in an ice-water bath for 35 minutes, this solution was poured into a mixture of ethyl acetate (150 ml) and 1N hydrochloric acid (150 ml). The organic phase was separated and washed with 1N hydrochloric acid (100 ml), saturated aqueous sodium bicarbonate (100 ml), and brine (100 ml). Dryness over magnesium sulfate and evaporation gave methyl (3R)-3-methanesulfonyloxyhexadecanoate (6.77 g).

NMR (CDCL₃, δ): 5.04 (1H, m), 3.72 (3H, s), 3.02 (3H, m), 2.78 (1H, dd, J=16, 8 Hz), 2.65 (1H, dd, J=16, 5 Hz), 1.77 (2H, m), 1.15-1.55 (22H, m), 0.88 (3H, t, J=7 Hz)

Preparation 18

A solution of methyl (3R)-3-methanesulfonyloxyhexadecanoate (6.77 g) and sodium azide (2.33 g) in dimethylformamide (60 ml) was heated to 60° C. for 40 minutes. This solution was poured into a mixture of ethyl acetate (300 ml) and water (500 ml). The organic phase was separated and washed with water (500 ml) and brine (300 ml). The resulting solution was dried over magnesium sulfate and evaporated to dryness to give methyl (3S)-3-azidohexadecanoate and some by-products. This crude product (5.0 g) was used in the next step without any further purification.

Preparation 19

Methyl (3S)-3-azidohexadecanoate (5.0 g) in methanol (25 ml) was hydrogenated over 10% palladium on carbon (0.50 g) under atmospheric pressure of hydrogen for 4 hours at room temperature. Then, the catalyst was filtered off with celite and the filtrate was concentrated under reduced pressure. The residue was dissolved with 4N hydrogen chloride in ethyl acetate (20 ml), evaporated, and triturated with diisopropyl ether (20 ml) to give methyl (3S)-3-aminohexadecanoate hydrochloride (930 mg).

NMR (CDCl₃, δ): 3.75 (3H, s), 3.60 (1H, m), 2.74-2.93 (2H, m), 1.57-1.98 (4H, m), 1.14-1.51 (20H, m), 0.87 (3H, t, J=7 Hz)

Preparation 20

To a suspension of methyl (3S)-3-aminohexadecanoate hydrochloride (890 mg) in water (1.8 ml) was added formalin (0.67 ml) and cyclopentadiene (1.14 ml) successively. The mixture was sonicated for 15 minutes, and stirred for 2.0 hours. The resulting mixture was washed with n-hexane, made basic with saturated sodium bicarbonate, and extracted with chloroform (×3). The combined organic phase was dried over magnesium sulfate, and concentrated under reduced pressure. To the residue in dichloromethane (12 ml) and trifluoroacetic acid (12 ml) was added triethylsilane (1.32 ml). This mixture was stirred overnight, and evaporated. This residue was dissolved in ethyl acetate (30 ml), washed with saturated aqueous sodium bicarbonate (20 ml), and dried over magnesium sulfate. After evaporation, the residue was purified on a silica gel (20 cc) to give methyl (3S)-3-(methylamino)hexadecanoate (686 mg).

NMR (CDCl₃, δ): 3.69 (3H, s), 2.98 (1H, m), 2.89 (1H, br s), 2.50 (2H, m), 2.45 (3H, s), 1.18-1.63 (24H, m), 0.87 (3H, t, J=7 Hz)

Preparation 21

Methyl 4-(3-benzo[b]furanyl)-3-oxobutanoate was obtained according to a similar manner to that of Preparation 1.

NMR (CDCl₃, δ): 7.64 (1H, s), 7.43-7.57 (2H, m), 7.21-7.36 (2H, m), 3.92 (2H, s), 3.71 (3H, s), 3.52 (2H, s)

Preparation 22

Methyl (3S)-4-(3-benzo[b]furanyl)-3-hydroxybutanoate was obtained according to a similar manner to that of Preparation 3.

NMR (CDCl₃, δ): 7.58 (1H, d, J=7 Hz), 7.52 (1H, s), 7.47 (1H, d, J=7 Hz), 7.19-7.34 (2H, m), 4.39 (1H, m), 3.68 (3H, s), 2.80-3.08 (3H, m), 2.42-2.65 (2H, m)

Preparation 23

To an ice-cooled solution of methyl (3S)-4-(3-benzo[b]furanyl)-3-hydroxybutanoate (250 mg) in methanol (2 ml) was added 1N aqueous sodium hydroxide (1.1 ml). This solution was stirred at room temperature overnight. Then it was diluted with water (20 ml), washed with diethyl ether (10 ml), acidified with 1N hydrochloric acid (1.4 ml), extracted with ethyl acetate (10 ml×3), and dried over magnesium sulfate. After evaporation, the residue was dissolved in dimethylformamide (2 ml). To this solution, HOBt (1-hydroxybenzotriazole) (136 mg), WSCD.HCl [1-ethyl-3-(3-dimethylaminopropyl)carbodiimide hydrochloride] (193 mg) was added successively. After 30 minutes, 28% ammonium hydroxide (91 μl) was added, and the mixture was stirred overnight. Then, the resulting mixture was diluted with 1N hydrochloric acid (20 ml) and extracted with ethyl acetate (20 ml). The organic phase was washed with 1N hydrochloric acid (20 ml), saturated aqueous sodium bicarbonate (20 ml) and brine. Dried over magnesium sulfate, evaporated to dryness, and chromatographed on a silica gel (20 cc) to give (3S)-4-(3-benzo[b]furanyl)-3-hydroxybutanamide (110 mg).

NMR (DMSO-d₆, δ): 7.76 (1H, s), 7.65 (1H, d, J=7 Hz), 7.53 (1H, d, J=8 Hz), 7.19-7.40 (3H, m), 6.82 (1H, br s), 4.94 (1H, d, J=6 Hz), 4.17 (1H, m), 2.79 (1H, dd, J=15, 5 Hz), 2.70 (1H, dd, J=15, 7 Hz), 2.11 (2H, d, J=7 Hz)

Preparation 24

To a suspension of methyl triphenylphosphoranylidene-acetate (2.45 g) in tetrahydrofuran (20 ml) was added 2-carboxybenzaldehyde (1.0 g) at 4° C. The resulting clear solution was stirred at room temperature for 30 minutes, and concentrated under reduced pressure. The residue was diluted with chloroform (20 ml) and extracted with saturated aqueous sodium bicarbonate (20 ml×2). The combined aqueous phase was washed with diethyl ether (20 ml), acidified with 1N hydrochloric acid (pH 5-6), and extracted with ethyl acetate (20 ml×2). The combined organic phase was washed with water (20 ml), brine (20 ml), dried over magnesium sulfate, and evaporated to dryness. The residue was triturated with diisopropyl ether to give methyl 2-carboxycinnamate (350 mg).

NMR (CDCl₃, δ): 8.55 (1H, d, J=16 Hz), 8.12 (1H, d, J=8 Hz), 7.56-7.67 (2H, m), 7.49 (1H, m), 6.34 (1H, d, J=16 Hz), 3.83 (3H, s)

Preparation 25

t-Butyl 2-carboxycinnamate was obtained according to a similar manner to that of Preparation 24.

NMR (CDCl₃, δ): 8.47 (1H, d, J=16 Hz), 8.10 (1H, d, J=8 Hz), 7.54-7.67 (2H, m), 7.47 (1H, m), 6.27 (1H, d, J=16 Hz), 1.55 (9H, s)

Preparation 26

A solution of methyl 2-carboxycinnamate (100 mg), palladium(II) acetate (5 mg) and potassium formate (108 mg) in dimethylformamide (1 ml) was stirred at 60° C. under nitrogen flow. The mixture was diluted with saturated aqueous ammonium chloride (20 ml), and extracted with ethyl acetate (20 ml). The organic phase was washed with water (20 ml) and brine (20 ml), dried over magnesium sulfate, and evaporated to dryness. The residue was triturated with diisopropyl ether to give methyl 3-(2-carboxyphenyl)propionate (82 mg).

NMR (CDCl₃, δ): 8.06 (1H, m), 7.49 (1H, m), 7.33 (3H, m), 3.34 (2H, t, J=8 Hz), 2.72 (2H, t, J=8 Hz)

Preparation 27

t-Butyl 3-(2-carboxyphenyl)propionate was obtained according to a similar manner to that of Preparation 26.

NMR (CDCl₃, δ): 8.04 (1H, d, J=7 Hz), 7.47 (1H, t, J=7 Hz), 7.27 (2H, m), 3.29 (2H, t, J=7 Hz), 2.53 (2H, t, J=7 Hz), 1.42 (9H, s)

Preparation 28

3-(2-Carboxyphenyl)propionamide was obtained according to a similar manner to that of Preparation 7.

NMR (DMSO-d₆, δ): 7.77 (1H, d, J=8 Hz), 7.45 (1H, dd, J=7, 6 Hz), 7.17-7.38 (3H, m), 6.74 (1H, br s), 3.11 (2H, t, J=8 Hz), 2.37 (2H, t, J=8 Hz)

Preparation 29

In a three-necked flask, under nitrogen flow, was placed magnesium turnings (1.26 g). In this flask, was added a solution of 1-bromohexane (8.59 g) in tetrahydrofuran (100 ml) dropwise. When the addition was completed, the whole was stirred for 30 minutes. The resulting mixture was added to an ice-cooled mixture of 4-bromobenzyl bromide (10.0 g) in tetrahydrofuran (100 ml) and 0.1M dilithium tetrachlorocuprate in tetrahydrofuran (10 ml). This mixture was stirred at 4° C. for 1.5 hours and at room temperature overnight. Then, it was poured into a mixture of ice and 1N hydrochloric acid (300 ml), and extracted with diethyl ether (300 ml). The etheral solution was washed with water (300 ml), saturated aqueous sodium bicarbonate (150 ml), and brine, dried over magnesium sulfate, and concentrated under reduced pressure. The residue was purified on a silica gel (200 cc) eluting with n-hexane to give 1-bromo-4-heptylbenzene (7.61 g).

NMR (CDCl₃, δ): 7.38 (2H, d, J=8 Hz), 7.04 (2H, d, J=8 Hz), 2.55 (2H, dd, J=7, 8 Hz), 1.48-1.67 (2H, m), 1.17-1.38 (8H, m), 0.88 (3H, t, J=7 Hz)

Preparation 30

1-Allyl-4-heptylbenzene was obtained according to a similar manner to that of Preparation 29.

NMR (CDCl₃, δ): 7.12 (2H, d, J=8 Hz), 7.08 (2H, d, J=8 Hz), 5.97 (1H, m), 5.02-5.13 (2H, m), 3.36 (2H, d, J=7 Hz), 2.58 (2H, t, J=8 Hz), 1.60 (2H, m), 1.19-1.40 (8H, m), 0.88 (3H, t, J=7 Hz)

Preparation 31

To a mixture of 1-allyl-4-heptylbenzene (2.4 g), acetone (40 ml) and water (40 ml) was added sodium metaperiodate (11.9 g) and potassium permanganate (70 mg). This mixture was stirred at room temperature overnight. Then, the mixture was filtered and the filtrate was concentrated under reduced pressure. The resulting aqueous solution was extracted with ethyl acetate (60 ml), and the organic phase was washed with water (60 ml) and brine (30 ml). This solution was dried over magnesium sulfate, and evaporated to dryness. The residue was purified on a silica gel (40 cc) eluting with 0%-5% methanol in chloroform to give 1-allyl-4-heptylbenzene (1.82 g) and 2-(4-heptylphenyl)acetic acid (300 mg).

NMR (CDCl₃, δ): 7.11-7.23 (4H, m), 3.62 (2H, s), 2.57 (2H, dd, J=8, 7 Hz), 1.59 (2H, m), 1.18-1.42 (8H, m), 0.87 (3H, t, J=7 Hz)

Preparation 32

Methyl (3S)-4-(4-heptylphenyl)-3-hydroxybutanoate was obtained according to a similar manner to that of Preparation 3.

NMR (CDCl₃, δ): 7.12 (4H, s), 4.25 (1H, m), 3.69 (3H, s), 2.70-2.88 (3H, m), 2.39-2.61 (4H, m), 1.59 (2H, m), 1.20-1.39 (8H, m), 0.87 (3H, t, J=7 Hz)

Preparation 33

Methyl 4-(4-heptylphenyl)-3-oxobutanoate was obtained according to a similar manner to that of Preparation 1.

NMR (CDCl₃, δ): 7.15 (2H, d, J=8 Hz), 7.10 (2H, d, J=8 Hz), 3.78 (2H, s), 3.70 (3H, s), 3.45 (2H, s), 2.58 (2H, m), 1.59 (2H, m), 1.20-1.40 (8H, m), 0.88 (3H, t, J=7 Hz)

Preparation 34

(3S)-4-(4-Heptylphenyl)-3-hydroxybutanamide was obtained according to a similar manner to that of Preparation 7.

NMR (CDCl₃, δ): 7.12 (4H, s), 5.88 (1H, br s), 5.41 (1H, br s), 4.23 (1H, m), 3.24 (1H, d, J=3 Hz), 2.84 (1H, dd, J=14, 7 Hz), 2.76 (1H, dd, J=14, 7 Hz), 2.57 (2H, dd, J=8, 7 Hz), 2.44 (1H, dd, J=15, 3 Hz), 2.33 (1H, dd, J=15, 8 Hz), 1.51-1.67 (2H, m), 1.18-1.40 (8H, m), 0.88 (3H, t, J=7 Hz)

### EXAMPLE 1

(2S)-5-Benzyloxycarbonyl-2-(tert-butoxycarbonylamino)pentanoic acid dicyclohexylammonium salt (1.00 g) was suspended with ethyl acetate and the mixture was washed with 0.5N sulfuric acid, water and brine, successively. The organic layer was dried over magnesium sulfate and concentrated in vacuo. The residue was dissolved in methylene chloride (10 ml) and to this was added DMAP (N,N-dimethylaminopyridine) (469 mg), PyBOP (benzotriazole-1-yloxy-tris-pyrrolidinophosphonium hexafluorophosphate) (1.07 g) and (3S)-3-hydroxy-4-(2-naphthyl)butanamide (430 mg) at room temperature. After being stirred overnight at the same temperature, the mixture was diluted with ethyl acetate and washed with 1N hydrochloric acid, aqueous ammonium chloride, aqueous sodium bicarbonate and brine, successively. The organic layer was dried over magnesium sulfate and concentrated in vacuo. The residue was purified by silica gel column chromatography (2% methanol in chloroform, as an eluent) to give (3S)-3-[(2S)-5-benzyloxycarbonyl-2-(tert-butoxycarbonylamino)pentanoyl]oxy-4-(2-naphthyl)butanamide (0.96 g).

NMR (CDCl₃, δ): 7.80 (3H, m), 7.66 (1H, s), 7.44 (2H, m), 7.34 (9H, m), 5.86 (1H, br s), 5.54 (1H, m), 5.30 (1H, br s), 5.06 (2H, s), 5.00 (1H, d, J=10 Hz), 4.18 (1H, m), 3.14 (2H, dd, J=8.0, 3.0 Hz), 2.46 (2H, m), 2.22 (2H, m), 1.5-1.6 (4H, m), 1.42 (9H, s)

The following compounds (Examples 2 to 5) were obtained according to a similar manner to that of Example 1.

### EXAMPLE 2

(3S)-4-(3-Benzo[b]furanyl)-3-[(2S)-5-benzyloxycarbonyl-2-(tert-butoxycarbonylamino)pentanoyl]oxybutanamide

NMR (CDCl₃, δ): 7.64 (1H, m), 7.53 (1H, s), 7.48 (1H, d, J=8 Hz) 7.22-7.41 (7H, m), 5.82 (1H, br s), 5.64 (1H, m), 5.27 (1H, br s), 5.10 (2H, s), 4.97 (1H, m), 4.22 (1H, m), 3.10 (2H, d, J=6 Hz), 2.50 (2H, d, J=7 Hz), 2.35 (2H, m), 1.62-1.84 (4H, m), 1.44 (9H, s)

### EXAMPLE 3

(3S)-3-[(2S)-5-Benzyloxycarbonyl-2-(tert-butoxycarbonylamino)pentanoyl]oxy-4-(4-heptylphenyl)butanamide

NMR (CDCl₃, δ): 7.30-7.39 (5H, m), 7.09 (4H, s), 5.82 (1H, br s), 5.43 (1H, m), 5.26 (1H, br s), 5.10 (2H, s), 5.01 (2H, s), 4.22 (1H, m), 2.97 (1H, dd, J=14, 7 Hz), 2.89 (1H, dd, J=14, 7 Hz), 2.55 (2H, dd, J=8, 7 Hz), 2.32-2.45 (4H, m), 1.50-1.72 (6H, m), 1.44 (9H, s), 1.21-1.36 (8H, m), 0.88 (3H, t, J=7 Hz)

### EXAMPLE 4

(3S)-3-[(2S)-5-Benzyloxycarbonyl-2-tert-butoxycarbonylaminopentanoyl]oxy-6-nonyloxyhexanamide NMR (CDCl₃, δ): 7.31-7.42 (5H, m), 5.90 (1H, br s), 5.25-5.34 (2H, m), 5.11 (2H, s), 5.05 (1H, d, J=8 Hz), 4.23 (1H, m), 3.40 (2H, t, J=6 Hz), 3.37 (2H, t, J=7 Hz), 2.47 (2H, d, J=6 Hz), 2.41 (2H, m), 1.49-1.92 (6H, m), 1.44 (9H, s), 1.21-1.37 (12H, m), 0.88 (3H, t, J=7 Hz)

### EXAMPLE 5

(3S)-3-[N-Methyl-{(2S)-5-benzyloxycarbonyl-2-(tert-butoxycarbonylamino)pentanoyl}amino]hexadecanamide

NMR (CDCl₃, δ): 7.25-7.4 (5H, m), 6.22 (1H, br s), 5.40 (1H, br s), 5.25 (1H, d, J=8.0 Hz), 5.10 (2H, s), 4.75 (1H, m), 4.52 (1H, m), 2.90 (3H, s), 2.3-2.5 (4H, m), 1.45-1.75 (6H, m), 1.43 (9H, s), 1.15-1.35 (22H, m), 0.88 (3H, t, J=7.5 Hz)

ESI-MS: 618 [M+H]

Preparation 35

(3S)-3-[(2S)-5-Benzyloxycarbonyl-2-(tert-butoxycarbonylamino)pentanoyl]oxy-4-(2-naphthyl)butanamide (0.94 g) was dissolved in 4N hydrogen chloride in ethyl acetate (50 ml) at room temperature. After being stirred for 1 hour at the same temperature, the mixture was diluted with ethyl acetate and the resulting solid was collected by filtration to give (3S)-3-[(2S)-2-amino-5-benzyloxycarbonylpentanoyl]oxy-4-(2-naphthyl)butanamide hydrochloride (0.67 g).

NMR (DMSO-d₆, δ): 8.60 (3H, br s), 7.88 (3H, m), 7.80 (1H, s), 7.3-7.55 (9H, m), 6.90 (1H, br s), 5.50 (1H, m), 5.10 (2H, s), 3.94 (1H, m), 3.10 (2H, m), 2.42 (2H, d, J=7.5 Hz), 2.28 (2H, m), 1.72 (2H, m), 1.54 (2H, m)

The following compounds (Preparations 36 to 41) were obtained according to a similar manner to that of Preparation 35.

Preparation 36

(3S)-4-(3-Benzo[b]furanyl)-3-[(2S)-5-benzyloxycarbonyl-2-aminopentanoyl]oxybutanamide

NMR (DMSO-d₆, δ): 8.52 (2H, br s), 7.88 (1H, s), 7.72 (1H, dd, J=6, 3 Hz), 7.56 (1H, d, J=8 Hz), 7.47 (1H, br s), 7.21-7.41 (7H, m), 6.91 (1H, br s), 5.48 (1H, m), 5.08 (2H, s), 3.98 (1H, m), 2.96-3.09 (2H, m), 2.45 (2H, d, J=6 Hz), 2.36 (2H, t, J=7 Hz), 1.47-1.89 (4H, m)

Preparation 37

(3S)-3-[(2S)-5-Benzyloxycarbonyl-2-aminopentanoyl]oxy-4-(4-heptylphenyl)butanamide hydrochloride

NMR (DMSO-d₆, δ): 8.42 (2H, br s), 7.43 (1H, br s), 7.31-7.41 (5H, m), 7.11 (4H, s), 6.87 (1H, br s), 5.38 (1H, m), 5.07 (2H, s), 3.97 (1H, m), 2.87 (2H, m), 2.29-2.40 (4H, m), 1.42-1.83 (6H, m), 1.16-1.35 (8H, m), 0.85 (3H, m)

Preparation 38

(3S)-3-[(2S)-5-Benzyloxycarbonyl-2-aminopentanoyl]oxy-6-nonyloxyhexanamide hydrochloride

NMR (DMSO-d₆, δ): 8.42 (2H, br s), 7.45 (1H, br s), 7.28-7.41 (5H, m), 6.87 (1H, br s), 5.23 (1H, m), 5.09 (2H, s), 3.99 (2H, s), 3.25-3.36 (4H, m), 2.31-2.44 (4H, m), 1.35-1.90 (10H, m), 1.10-1.32 (12H, m), 0.85 (3H, m)

Preparation 39

(3S)-3-[(2S)-2-Amino-5-benzyloxycarbonylpentanoyl]amino-4-(2-naphthyl)butanamide hydrochloride

NMR (CDCl₃, δ): 8.66 (1H, br s), 8.46 (3H, br s), 7.56 (5H, br s), 7.1-7.4 (9H, m), 4.86 (2H, s), 4.42 (1H, m), 4.06 (1H, m), 3.22 (1H, m), 2.94 (1H, m), 2.76 (1H, m), 2.36 (1H, m), 1.16-2.0 (6H, m)

Preparation 40

(3S)-3-[N-Methyl-{(2S)-2-amino-5-benzyloxycarbonylpentanoyl}amino]hexadecanamide hydrochloride

NMR (CDCl₃, δ): 8.3-8.5 (3H, m), 8.14 (1H, br s), 7.2-7.4 (5H, m), 7.14 (1H, br s), 5.08 (2H, s), 4.85 (1H, m), 4.14 (1H, m), 2.85 (3H, s), 1.4-2.8 (10H, m), 1.1-1.3 (22H, m), 0.88 (3H, t, J=7.5 Hz)

Preparation 41

(3S)-3-[(2S)-5-Benzyloxycarbonyl-2-aminopentanoyl]aminohexadecanamide hydrochloride

NMR (DMSO-d₆, δ): 8.25 (1H, d, J=8 Hz), 8.12 (2H, m), 7.29-7.34 (6H, m), 6.80 (1H, br s), 5.08 (2H, s), 4.04 (1H, m), 3.80 (1H, m), 2.38 (2H, m), 2.21 (2H, d, J=7 Hz), 1.51-1.86 (4H, m), 1.10-1.47 (24H, m), 0.85 (3H, t, J=7 Hz)

Preparation 42

(2S)-5-Benzyloxycarbonyl-2-(tert-butoxycarbonylamino)pentanoic acid dicyclohexylammonium salt (1.07 g) was suspended with ethyl acetate and the mixture was washed with 0.5N sulfuric acid, water and brine, successively. The organic layer was dried over magnesium sulfate and concentrated in vacuo. The residue was dissolved in methylene chloride (10 ml) and to this was added DMAP (469 mg), PyBOP (1.05 g) and (3S)-4-(6-ethyl-2-naphthyl)-3-hydroxybutanamide (0.47 g) at room temperature. After being stirred overnight at the same temperature, the mixture was diluted with ethyl acetate and washed with 1N hydrochloric acid, aqueous ammonium chloride, aqueous sodium bicarbonate and brine, successively. The organic layer was dried over magnesium sulfate and concentrated in vacuo. The residue was dissolved in 4N hydrogen chloride in ethyl acetate (20 ml) at room temperature. After being stirred for 1 hour at the same temperature, the mixture was diluted with ethyl acetate and the resulting solid was collected by filtration to give (3S)-3-[(2S)-2-amino-5-benzyloxycarbonylpentanoyl]oxy-4-(6-ethyl-2-naphthyl)butanamide hydrochloride (0.70 g).

NMR (DMSO-d₆, δ): 8.52 (3H, br s), 7.80 (2H, dd, J=8.0, 6.0 Hz), 7.72 (1H, s), 7.66 (1H, s), 7.48 (1H, br s), 7.3-7.4 (7H, m), 6.90 (1H, br s), 5.50 (1H, m), 5.08 (2H, s), 3.94 (1H, m), 3.06 (2H, m), 2.74 (2H, q, J=7.5 Hz), 2.40 (2H, d, J=7.5 Hz), 2.28 (2H, m), 1.4-1.8 (4H, m), 1.24 (3H, t, J=7.5 Hz)

### EXAMPLE 6

To a stirring solution of (3S)-3-[(2S)-2-amino-5-benzyloxycarbonylpentanoyl]oxy-4-(6-ethyl-2-naphthyl)butanamide hydrochloride (0.20 g), 1-benzylindole-3-carboxylic acid (105 mg) and HOBt (62 mg) in DMF (2 ml) was added WSCD (71 mg) at 0° C. After being stirred at room temperature overnight, the mixture was diluted with ethyl acetate and washed with 1N hydrochloric acid, aqueous ammonium chloride, aqueous sodium bicarbonate and brine, successively. The organic layer was dried over magnesium sulfate and concentrated in vacuo to give (3S)-3-[(2S)-2-(1-benzylindol-3-ylcarbonylamino)-5-benzyloxycarbonylpentanoyl]oxy-4-(6-ethyl-2-naphthyl)butanamide (0.26 g).

NMR (CDCl₃, δ): 8.20 (1H, s), 8.1 (2H, m), 7.1-7.8 (19H, m), 6.66 (1H, d, J=10 Hz), 6.1 (1H, br s), 5.6 (1H, m), 5.34 (2H, s), 5.08 (2H, s), 4.7 (1H, m), 3.1 (2H, m), 2.74 (2H, q, J=7.5 Hz), 2.1-2.6 (4H, m), 1.4-1.9 (4H, m), 1.28 (3H, t, J=7.5 Hz)

ESI-MS: 724 [M+H]

The following compounds (Examples 7 to 20) were obtained according to a similar manner to that of Example 6.

### EXAMPLE 7

(3S)-3-[(2S)-2-(1-Benzylindol-2-ylcarbonylamino)-5-benzyloxycarbonylpentanoyl]oxy-4-(2-naphthyl)butanamide NMR (CDCl₃, δ): 7.72 (4H, m), 7.62 (1H, s), 7.1-7.45 (14H, m), 7.02 (3H, m), 6.92 (1H, d, J=7.5 Hz), 5.82 (1H, br s), 5.78 (2H, ABq), 5.54 (1H, m), 5.20 (1H, br s), 5.10 (2H, s), 4.54 (1H, m), 3.08 (2H, m), 2.05-2.45 (4H, m), 1.70 (2H, m), 1.44 (2H, m)

ESI-MS 696 [M+H]

### Example 8

(3S)-3-[(2S)-2-(1-Benzylindol-3-ylcarbonylamino)-5-benzyloxycarbonylpentanoyl]oxy-4-(2-naphthyl)butanamide NMR (CDCl₃, δ): 8.06 (1H, m), 7.7 (4H, m), 7.62 (1H, s), 7.2-7.4 (15H, m), 7.16 (1H, d, J=70.5 Hz), 6.66 (1H, d, J=7.5 Hz), 6.10 (1H, br s), 5.60 (1H, m), 5.34 (2H, s), 5.26 (1H, br s), 5.08 (2H, s), 4.66 (1H, m), 3.14 (2H, d, J=7.5 Hz), 2.52 (2H, ABX), 2.1-2.3 (2H, m), 1.65-1.85 (2H, m), 1.45-1.6 (2H, m)

ESI-MS: 696 [M+H]

### EXAMPLE 9

(3S)-3-[(2S)-5-Benzyloxycarbonyl-2-(2-quinolylcarbonylamino)pentanoyl]oxy-4-(2-naphthyl)butanamide

NMR (CDCl₃, δ): 8.70 (1H, d, J=10 Hz), 7.3-8.5 (18H, m), 5.92 (1H, br s), 5.62 (1H, m), 5.32 (1H, br s), 5.10 (2H, s), 4.74 (1H, m), 3.16 (2H, d, J=7.5 Hz), 2.52 (2H, m), 2.30 (2H, m), 1.6-2.0 (4H, m)

ESI-MS 618 [M+H]

### EXAMPLE 10

(3S)-3-[(2S)-2-(3-Benzylnaphthalen-2-ylcarbonylamino)-5-benzyloxycarbonylpentanoyl]oxy-4-(2-naphthyl)butanamide NMR (CDCl₃, δ): 7.05-7.86 (23H, m), 6.34 (1H, d, J=8.0 Hz), 5.80 (1H, br s), 5.58 (1H, m), 5.28 (1H, br s), 5.04 (2H, s), 4.50 (1H, m), 4.34 (2H, ABq), 3.16 (2H, d, J=7.5 Hz), 2.48 (2H, m), 2.16 (2H, m), 1.44-1.70 (2H, m), 1.28-1.40 (2H, m)

ESI-MS: 707 [M+H]

### EXAMPLE 11

(3S)-4-(2-Naphthyl)-3-[(2S)-5-benzyloxycarbonyl-2-[2-(2-methoxycarbonylethyl)benzoylamino]pentanoyl]oxybutanamide

NMR (CDCl₃, δ): 7.68-7.82 (3H, m), 7.65 (1H, br s), 7.21-7.46 (12H, m), 6.93 (1H, d, J=8 Hz), 6.02 (1H, br s), 5.61 (1H, m), 5.31 (1H, br s), 5.08 (2H, s), 4.59 (1H, m), 3.59 (3H, s), 3.18 (2H, m), 3.05 (2H, m), 2.72 (2H, m), 2.54 (2H, d, J=7 Hz), 2.16-2.34 (2H, m), 1.48-1.86 (4H, m)

### EXAMPLE 12

(3S)-4-(2-Naphthyl)-3-[(2S)-5-benzyloxycarbonyl-2-[2-(2-t-butoxycarbonylethyl)benzoylamino]pentanoyl]oxybutanamide

NMR (CDCl₃, δ): 7.68-7.81 (3H, m), 7.65 (1H, br s), 7.10-7.48 (13H, m), 6.10 (1H, br s), 5.60 (1H, m), 5.28 (1H, br s), 5.06 (2H, s), 4.56 (1H, m), 3.18 (2H, m), 2.93-3.13 (2H, m), 2.50-2.69 (4H, m), 2.22 (2H, m), 1.50-1.84 (4H, m), 1.36 (9H, s)

### EXAMPLE 13

(3S)-4-(2-Naphthyl)-3-[(2S)-5-benzyloxycarbonyl-2-[2-(2-carbamoylethyl)benzoylamino]pentanoyl]oxybutanamide

NMR (DMSO-d₆, δ): 8.85 (1H, d, J=7 Hz), 7.79-7.88 (3H, m), 7.75 (1H, br s), 7.18-7.50 (12H, m), 6.68 (1H, br s), 6.82 (1H, br s), 5.42 (1H, m), 5.06 (2H, s), 4.31 (1H, m), 3.13 (1H, dd, J=14 Hz, 5 Hz), 3.02 (1H, dd, J=14, 6 Hz), 2.92 (2H, dd, J=16, 8 Hz), 2.33-2.46 (4H, m), 2.23 (2H, m), 1.44-1.73 (4H, m)

### EXAMPLE 14

(3S)-3-[(2S)-5-Benzyloxycarbonyl-2-(2-quinolylcarbonylamino)pentanoyl]oxy-4-(4-heptylphenyl)butanamide

NMR (CDCl₃, δ): 8.69 (1H, d, J=8 Hz), 8.33 (1H, d, J=8 Hz), 8.26 (1H, d, J=8 Hz), 8.18 (1H, d, J=8 Hz), 7.90 (1H, d, J=8 Hz), 7.80 (1H, m), 7.75 (1H, m), 7.28-7.38 (5H, m), 7.11 (2H, d, J=8 Hz), 7.02 (1H, d, J=8 Hz), 5.85 (1H, br s), 5.49 (1H, m), 5.30 (1H, br s), 5.10 (2H, s), 4.76 (1H, m), 2.53-2.86 (2H, m), 2.35-2.53 (6H, m), 1.40-2.10 (6H, m), 1.15-1.34 (8H, m), 0.88 (3H, t, J=7 Hz)

### EXAMPLE 15

(3S)-4-(3-Benzo[b]furanyl)-3-[(2S)-5-benzyloxycarbonyl-2-(2-quinolylcarbonylamino)pentanoyl]oxybutanamide

NMR (CDCl₃, δ): 8.68 (1H, d, J=8 Hz), 8.34 (1H, d, J=8 Hz), 8.27 (1H, d, J=8 Hz), 8.19 (1H, d, J=8 Hz), 7.91 (1H, d, J=8 Hz), 7.81 (1H, d, J=8 Hz, 7 Hz), 7.62-7.71 (2H, m), 7.54 (1H, s), 7.43 (1H, m), 7.23-7.37 (7H, m), 5.86 (1H, br s), 5.58 (1H, m), 5.28 (1H, br s), 5.09 (2H, s), 4.75 (1H, m), 3.10 (2H, d, J=7 Hz), 2.53 (2H, m), 2.38 (2H, m), 1.64-2.03 (4H, m)

### EXAMPLE 16

(3S)-3-[(2S)-5-Benzyloxycarbonyl-2-(2-quinolylcarbonylamino)pentanoyl]oxy-6-nonyloxyhexanamide

NMR (CDCl₃, δ): 8.74 (1H, d, J=8 Hz), 8.32 (1H, d, J=8 Hz), 8.25 (1H, d, J=8 Hz), 8.16 (1H, d, J=9 Hz), 7.88 (1H, d, J=8 Hz), 7.77 (1H, t, J=8 Hz), 7.62 (1H, t, J=8 Hz), 7.28-7.37 (5H, m), 5.93 (1H, br s), 5.36 (1H, m), 5.28 (1H, br s), 5.11 (2H, s), 4.78 (1H, m), 3.30-3.41 (4H, m), 2.41-2.53 (4H, m), 1.45-2.15 (10H, m), 1.15-1.33 (12H, m), 0.86 (3H, t, J=7 Hz)

### EXAMPLE 17

(3S)-3-[(2S)-5-Benzyloxycarbonyl-2-(tert-butoxycarbonylamino)pentanoyl]amino-4-(2-naphthyl)butanamide

NMR (CDCl₃, δ): 7.74-7.82 (3H, m), 7.64 (1H, s), 7.3-7.5 (8H, m), 7.14 (1H, d, J=10 Hz), 5.76 (1H, br s), 5.56 (1H, br s), 5.08 (1H, d, J=8.0 Hz), 5.06 (2H, s), 4.50 (1H, m), 4.00 (1H, m), 3.16 (1H, dd, J=12.0, 7.5 Hz), 3.02 (1H, dd, J=12.0, 7.5 Hz), 2.42 (2H, ABX), 2.26 (2H, m), 1.6-1.75 (2H, m), 1.45-1.6 (2H, m), 1.4 (9H, s)

ESI-MS: 562 [M+H]

### EXAMPLE 18

(3S)-3-[(2S)-2-(1-Benzylindol-3-ylcarbonylamino)-5-benzyloxycarbonylpentanoyl]amino-4-(2-naphthyl)butanamide

NMR (DMSO-d₆, δ): 8.26 (1H, s), 8.20 (1H, d, J=8.0 Hz), 8.02 (1H, d, J=8.0 Hz), 7.86 (1H, d, J=8.0 Hz), 7.64-7.74 (4H, m), 7.54 (1H, d, J=8.0 Hz), 7.1-7.4 (16H, m), 6.84 (1H, br s), 5.46 (2H, s), 5.06 (2H, s), 4.44 (1H, m), 4.30 (1H, m), 2.82-3.0 (2H, m), 2.2-2.4 (4H, m), 1.4-1.8 (4H, m)

ESI-MS: 695 [M+H]

### EXAMPLE 19

(3S)-3-[N-Methyl-{(2S)-5-benzyloxycarbonyl-2-(2-quinolylcarbonylamino)pentanoyl}amino]hexadecanamide

NMR (CDCl₃, δ): 8.90 (1H, d, J=8.0 Hz), 8.1-8.35 (3H, m), 7.85 (1H, d, J=8.0 Hz), 7.75 (1H, t, J=8.0 Hz), 7.62 (1H, t, J=8.0 Hz), 7.25-7.35 (5H, m), 6.32 (1H, br s), 5.52 (1H, br s), 5.16 (1H, m), 5.10 (2H, s), 4.82 (1H, m), 3.00 (3H, s), 2.4-2.55 (4H, m), 1.4-2.05 (6H, m), 1.05-1.4 (22H, m), 0.88 (3H, t, J=70.5 Hz)

ESI-MS 673 [M+H]

### EXAMPLE 20

(3S)-3-[(2S)-5-Benzyloxycarbonyl-2-(2-quinolylcarbonylamino)pentanoyl]aminohexadecanamide

NMR (CDCl₃, δ): 8.71 (1H, d, J=8 Hz), 8.32 (1H, d, J=8 Hz), 8.25 (1H, d, J=8 Hz), 8.15 (1H, d, J=9 Hz), 7.88 (1H, d, J=8 Hz), 7.78 (1H, t, J=8 Hz), 7.63 (1H, t, J=8 Hz), 7.26-7.36 (5H, m), 6.82 (1H, d, J=8 Hz), 6.06 (1H, br s), 5.35 (1H, br s), 5.12 (2H, s), 4.62 (1H, m), 4.18 (1H, m), 2.37-2.54 (4H, m), 1.48-2.18 (6H, m), 1.02-1.36 (22H, m), 0.87 (3H, t, J=7 Hz)

### EXAMPLE 21

To a stirring solution of (3S)-3-[(2S)-2-(1-benzylindol-3-ylcarbonylamino)-5-benzyloxycarbonylpentanoyl]oxy-4-(6-ethyl-2-naphthyl)butanamide (0.24 g) and anisole (717 mg) in methylene chloride (2.5 ml) was added aluminum chloride (442 mg) in nitromethane (2.5 ml) at room temperature. After being stirred for two hours at the same temperature, the mixture was diluted with ethyl acetate and washed with 1N hydrochloric acid, water and brine, successively. The organic layer was dried over magnesium sulfate and concentrated in vacuo. The resulting solid was triturated with ethyl ether to give (3S)-3-[(2S)-2-(1-benzylindol-3-ylcarbonylamino)-5-carboxypentanoyl]oxy-4-(6-ethyl-2-naphthyl)butanamide (113 mg).

NMR (DMSO-d₆, δ): 8.28 (1H, s), 8.16 (2H, dd, J=8.0, 3.0 Hz), 7.1-7.8 (14H, m), 6.86 (1H, br s), 5.50 (2H, s), 5.40 (1H, m), 4.40 (1H, m), 3.04 (2H, ABX), 2.74 (2H, a, J=7.5 Hz), 2.34 (2H, d, J=7.5 Hz), 2.18 (2H, t, J=7.5 Hz), 1.5-1.8 (4H, m), 1.24 (3H, t, J=7.5 Hz)

The following compounds (Examples 22 to 32) were obtained according to a similar manner to that of Example 21.

### EXAMPLE 22

(3S)-3-[(2S)-2-(1-Benzylindol-3-ylcarbonylamino)-5-carboxypentanoyl]oxy-4-(2-naphthyl)butanamide

NMR (DMSO-d₆, δ): 8.28 (1H, s), 8.20 (2H, d, J=7.5 Hz), 7.8 (3H, m), 7.54 (1H, d, J=7.5 Hz), 7.1-7.6 (11H, m), 6.86 (1H, br s), 5.46 (2H, s), 5.40 (1H, m) 4.40 (1H, m), 3.05 (2H, ABX), 2.32 (2H, d, J=7.5 Hz), 2.16 (2H, t, J=70.5 Hz), 1.70 (2H, m), 1.55 (2H, m)

ESI-MS: 606 [M+H]

### EXAMPLE 23

(3S)-3-[(2S)-5-Carboxy-2-(2-quinolylcarbonylamino)pentanoyl]oxy-4-(2-naphthyl)butanamide

NMR (CDCl₃, δ): 8.72 (1H, d, J=10 Hz), 7.15-8.3 (13H, m), 7.04 (1H, br s), 6.50 (1H, br s), 5.60 (1H, m), 4.76 (1H, m), 2.9-3.2 (2H, m), 2.44 (2H, m), 2.24 (2H, m), 1.35-2.1 (4H, m)

ESI-MS: 528 [M+H]

### EXAMPLE 24

(3S)-3-[(2S)-2-(3-Benzylnaphthalen-2-ylcarbonylamino)-5-carboxypentanoyl] oxy-4-(2-naphthyl)butanamide

NMR (DMSO-d₆, δ): 8.84 (1H, d, J=8.0 Hz), 7.06-7.98 (19H, m), 6.88 (1H, s), 5.42 (1H, m), 4.34 (1H, m), 4.28 (2H, ABq), 3.10 (2H, ABX), 2.36 (2H, d, J=7.5 Hz), 2.10 (2H, t, J=7.5 Hz), 1.4-1.75 (4H, m)

ESI-MS 617 [M+H]

### EXAMPLE 25

(3S)-4-(2-Naphthyl)-3-[(2S)-5-carboxy-2-[2-(2-methoxycarbonylethyl)benzoylamino]pentanoyl]oxybutanamide

NMR (DMSO-d₆, δ): 8.75 (1H, d, J=8 Hz), 7.80-7.91 (3H, m), 7.78 (1H, br s), 7.22-7.50 (8H, m), 6.86 (1H, br s), 5.41 (1H, m), 4.32 (1H, m), 3.50 (3H, s), 2.90-3.18 (4H, m), 2.63 (2H, dd, J=8, 7 Hz), 2.35 (2H, d, J=7 Hz), 2.13 (2H, t, J=7 Hz), 1.44-1.72 (4H, m)

### EXAMPLE 26

(3S)-4-(2-Naphthyl)-3-[(2S)-5-carboxy-2-[2-(2-carboxyethyl)benzoylamino]pentanoyl]oxybutanamide

NMR (DMSO-d₆, δ): 8.74 (1H, d, J=7 Hz), 7.80-7.92 (3H, m), 7.77 (1H, br s), 7.22-7.53 (8H, m), 6.87 (1H, br s), 5.42 (1H, m), 4.31 (1H, m), 3.15 (1H, dd, J=14, 6 Hz), 2.86-3.08 (3H, m), 2.57 (2H, m), 2.37 (2H, d, J=7 Hz), 2.13 (2H, t, J=7 Hz), 1.42-1.72 (4H,

### EXAMPLE 27

(3S)-4-(2-Naphthyl)-3-[(2S)-5-carboxy-2-[2-(2-carbamoylethyl)benzoylamino]pentanoyl]oxybutanamide

NMR (DMSO-d₆, δ): 8.86 (1H, d, J=7 Hz), 7.80-7.92 (3H, m), 7.75 (1H, br s), 7.15-7.53 (10H, m), 6.88 (1H, br s), 6.73 (1H, br s), 5.42 (1H, m), 4.32 (1H, m), 2.82-3.18 (4H, m), 2.35-2.47 (4H, m), 2.11 (2H, t, J=7 Hz), 1.42-1.77 (4H, m)

### EXAMPLE 28

(3S)-3-[(2S)-5-Carboxy-2-(2-quinolylcarbonylamino)pentanoyl]oxy-4-(4-heptylphenyl)butanamide

NMR (DMSO-d₆, δ): 8.95 (1H, d, J=8 Hz), 8.62 (1H, d, J=8 Hz), 8.20 (1H, d, J=9 Hz), 8.17 (1H, d, J=8 Hz), 8.12 (1H, d, J=8 Hz), 7.90 (1H, t, J=8 Hz), 7.74 (1H, t, J=8 Hz), 7.37 (1H, br s), 7.08 (1H, d, J=8 Hz), 6.92 (1H, d, J=8 Hz), 6.86 (1H, br s), 5.33 (1H, m), 4.51 (1H, m), 2.87 (1H, dd, J=14, 6 Hz), 2.78 (1H, dd, J=14, 7 Hz), 2.19-2.40 (4H, m), 1.84 (2H, m), 1.53 (2H, m), 1.36 (2H, m), 1.10-1.30 (8H, m), 0.83 (3H, t, J=7 Hz)

### EXAMPLE 29

(3S)-3-[(2S)-5-Carboxy-2-(2-quinolylcarbonylamino)pentanoyl]oxy-6-nonyloxyhexanamide

NMR (CDCl₃, δ): 8.84 (1H, d, J=8 Hz), 8.32 (1H, d, J=8 Hz), 8.26 (1H, d, J=8 Hz), 8.17 (1H, d, J=8 Hz), 7.88 (1H, d, J=8 Hz), 7.78 (1H, m), 7.63 (1H, m), 7.13 (1H, br s), 6.25 (1H, br s), 5.43 (1H, m), 4.83 (1H, m), 3.31-3.46 (4H, m), 2.34-2.77 (4H, m), 1.90-2.20 (2H, m), 1.40-1.88 (10H, m), 1.14-1.37 (12H, m), 0.86 (3H, t, J=7 Hz)

### EXAMPLE 30

(3S)-3-[(2S)-2-(1-Benzylindol-3-ylcarbonylamino)-5-carboxypentanoyl]amino-4-(2-naphthyl)butanamide

NMR (DMSO-d₆, δ): 8.24 (1H, s), 8.20 (1H, d, J=8.0 Hz), 8.04 (1H, d, J=8.0 Hz), 7.84 (1H, d, J=8.0 Hz), 7.64-7.74 (4H, m), 7.54 (1H, d, J=8.0 Hz), 7.1-7.4 (11H, m), 6.84 (1H, br s), 5.44 (2H, s), 4.42 (1H, m), 4.30 (1H, m), 2.8-3.0 (2H, m), 2.26 (2H, d, J=7.5 Hz), 2.16 (2H, t, J=7.5 Hz), 1.4-1.75 (4H, m)

ESI-MS: 605 [M+H]

### EXAMPLE 31

(3S)-3-[N-Methyl-{(2S)-5-carboxy-2-(2-quinolylcarbonylamino)pentanoyl}amino]hexadecanamide

NMR (CDCl₃, δ): 9.00 (1H, d, J=8.0 Hz), 8.26 (2H, ABq), 8.16 (1H, d, J=8.0 Hz), 7.86 (1H, d, J=8.0 Hz), 7.75 (1H, t, J=8.0 Hz), 7.60 (1H, t, J=8.0 Hz), 7.10 (1H, br s), 6.80 (1H, br s), 5.20 (1H, m), 5.10 (1H, m), 3.05 (3H, s), 2.58 (1H, dd, J=15.0, 5 Hz), 2.34-2.50 (3H, m), 1.4-2.1 (6H, m), 1.0-1.35 (22H, m), 0.86 (3H, t, J=7.5 Hz)

ESI-MS: 583 [M+H]

### EXAMPLE 32

(3S)-3-[(2S)-5-Carboxy-2-(2-quinolylcarbonylamino)pentanoyl]aminohexadecanamide

NMR (DMSO-d₆, δ): 8.75 (1H, d, J=9 Hz), 8.09 (1H, d, J=9 Hz), 8.17 (2H, d, J=8 Hz), 8.10 (1H, d, J=8 Hz), 8.07 (1H, d, J=9 Hz), 7.88 (1H, t, J=8 Hz), 7.73 (1H, t, J=8 Hz), 7.27 (1H, br s), 6.79 (1H, br s), 4.55 (1H, m), 4.06 (1H, m), 2.12-2.30 (4H, m), 1.32-1.91 (6H, m), 0.95-1.30 (18H, m), 0.83 (3H, t, J=7 Hz)

### EXAMPLE 33

A solution of (3S)-3-[(2S)-2-(1-benzylindol-2-ylcarbonylamino)-5-benzyloxycarbonylpentanoyl]oxy-4-(2-naphthyl)butanamide (0.22 g) in water (0.4 ml) and methanol (4 ml) was hydrogenated over 10% palladium on carbon (40 mg) under atmospheric pressure of hydrogen for two days at room temperature. Then the catalyst was filtered off with celite and filtrate was concentrated under reduced pressure. The residue was triturated with ether and chloroform to give (3S)-3-[(2S)-2-(1-benzylindol-2-ylcarbonylamino)-5-carboxypentanoyl]oxy-4-(2-naphthyl)butanamide (168 mg).

NMR (DMSO-d₆, δ): 8.92 (1H, d, J=8.0 Hz), 7.05-7.85 (18H, m), 6.86 (1H, br s), 5.80 (2H, ABq), 5.40 (1H, m), 4.32 (1H, m), 3.02 (2H, ABX), 2.50 (2H, s), 2.32 (2H, d, J=7.5 Hz), 2.15 (2H, t, J=7.5 Hz), 1.65-1.8 (2H, m), 1.4-1.65 (2H, m)

ESI-MS: 606 [M+H]

### EXAMPLE 34

A mixture of (3S)-4-(3-benzo[b]furanyl)-3-[(2S)-5-benzyloxycarbonyl-2-(2-quinolylcarbonylamino)pentanoyl]-oxybutanamide (184 mg), cyclohexene (0.31 ml), and 10% palladium on carbon (130 mg) in dioxane (1 ml) was refluxed for 2.5 hours. After filtration with celite, and filtrate was partitioned between ethyl acetate (20 ml) and water (20 ml). The organic phase was washed with brine, dried over magnesium sulfate, and concentrated in vacuo. The residue was triturated in diethyl ether (5 ml) to give (3S)-4-(3-benzo[b]furanyl)-3-[(2S)-5-carboxy-2-(2-quinolylcarbonylamino)pentanoyl]oxybutanamide (107 mg).

NMR (DMSO-d₆, δ): 9.02 (1H, d, J=8 Hz), 8.61 (1H, d, J=8 Hz), 8.21 (1H, d, J=8 Hz), 8.17 (1H, d, J=8 Hz), 8.12 (1H, d, J=8 Hz), 7.91 (1H, t, J=8 Hz), 7.70-7.79 (2H, m), 7.52 (1H, dd, J=7, 2 Hz), 7.41 (1H, br s), 7.12-7.34 (2H, m), 6.89 (1H, br s), 5.45 (1H, m), 4.53 (1H, m), 3.03 (2H, m), 2.42 (2H, m), 2.23 (2H, t, J=7 Hz), 1.86 (2H, m), 1.55 (2H, m)

### EXAMPLE 35

A solution of (3S)-4-(2-naphthyl)-3-[(2S)-5-benzyloxycarbonyl-2-{2-(2-t-butoxycarbonylethyl)benzoylamino}pentanoyl]oxybutanamide (160 mg) in 4N hydrogen chloride in ethyl acetate (1 ml) was stirred at room temperature for four hours. Then, the mixture was concentrated in vacuo, dissolved in ethyl acetate (20 ml), washed with water (20 ml×2) and brine (20 ml), and dried over magnesium sulfate. After evaporation, the residue was triturated in diisopropyl ether to give (3S)-4-(2-naphthyl)-3-[(2S)-5-benzyloxycarbonyl-2-{2-(2-carboxyethyl)benzoylamino}pentanoyl]oxybutanamide (91 mg).

NMR (CDCl₃, δ): 7.66-7.80 (3H, m), 7.60 (1H, br s), 7.17-7.46 (12H, m), 7.04 (1H, d, J=8 Hz), 6.33 (1H, br s), 6.21 (1H, br s), 5.60 (1H, m), 5.05 (2H, s), 4.62 (1H, m), 2.92-3.20 (4H, m), 2.50-2.69 (4H, m), 2.19-2.32 (2H, m), 1.45-1.81 (4H, m)

Preparation 43

(3R)-3-Hydroxyhexadecanamide was obtained according to a similar manner to that of Preparation 23.

NMR (DMSO-d6, δ): 7.26 (1H, br s), 6.88 (1H, br s), 4.58 (1H, d, J=5 Hz), 3.75 (1H, m), 2.11 (2H, d, J=6 Hz), 1.14-1.40 (24H, m), 0.84 (3H, t, J=7 Hz)

Preparation 44

To a solution of (3R)-3-hydroxyhexadecanamide (6.4 g) and triethylamine (6.57 ml) in dichloromethane (130 ml) and dimethylsulfoxide (130 ml) was added methanesulfonyl chloride (2.74 ml). This mixture was stirred at room temperature for 6 hours, and the resulting precipitate was filtered off. The liquor was concentrated, and dissolved in ethyl acetate (500 ml). This solution was washed with 1N-hydrochloric acid (800 ml×2), saturated sodium carbonate (500 ml), and brine (200 ml). Drying over magnesium sulfate and concentration, the residue was chromatographed on a silica gel, eluting with a mixture of dichloromethane and methanol (50:1) to give (3R)-3-(methanesulfonyloxy)hexadecanamide (4.16 g).

NMR (CDCl₃, δ): 5.68 (1H, br s), 5.45 (1H, br s), 5.04 (1H, m), 3.04 (3H, s), 2.62 (2H, d, J=7 Hz), 1.81 (2H, m), 1.35-1.50 (22H, m), 0.87 (3H, t, J=7 Hz)

Preparation 45

A solution of (3R)-3-(methanesulfonyloxy)hexadecanamide (4.16 g) and sodium azide (1.55 g) in dimethylformamide (50 ml) was heated at 60° C. for 2 hours. The cooled mixture was partitioned between ethyl acetate (300 ml) and water (500 ml). The organic phase was washed with water (300 ml), and brine (200 ml). Dried, concentrated under vacuum, the residue was purified on a silica gel (200 cc) to give (3S)-3-azidohexadecanamide (2.20 g).

NMR (CDCl₃, δ): 5.62 (1H, br s), 5.40 (1H, br s), 3.85 (1H, m), 2.42 (1H, dd, J=15 Hz, 6 Hz), 2.34 (1H, dd, J=15 Hz, 8 Hz), 1.18-1.64 (24H, m), 0.88 (3H, t, J=7 Hz)

Preparation 46

A mixture of (3S)-3-azidohexadecanamide (2.18 g) and 10% palladium on carbon (320 mg) was hydrogenated at atmospheric pressure for 10 hours. The catalyst was filtered off with celite, and the liquor was concentrated under reduced pressure. The residue was triturated in diisopropyl ether (30 ml) to give (3S)-3-aminohexadecanamide.

NMR (DMSO-d6, δ): 7.36 (1H, br s), 6.72 (1H, br s), 2.89 (1H, m), 2.08 (1H, dd, J=14 Hz, 5 Hz), 1.94 (1H, dd, J=14 Hz, 8 Hz), 1.13-1.52 (24H, m), 0.85 (3H, t, J=7 Hz)

(3S)-3-aminohexadecanamide thus obtained was dissolved in 4N hydrogen chloride in ethyl acetate (15 ml), and concentrated under reduced pressure. The residue was triturated in diisopropyl ether to give (3S)-3-aminohexadecanamide hydrochloride (1.55 g).

Preparation 47

To an ice-cooled solution of (3S)-3-aminohexadecanamide (300 mg) and propionaldehyde (71 μl) in methanol (6 ml) was added sodium cyanoborohydride (61 mg). After 2 hours, propionaldehyde (71 μl) and sodium cyanoborohydride (61 mg) was added. The solution was stirred overnight. Then, the solvent was evaporated, diluted with water (20 ml), and extracted with chloroform (20 ml). The organic phase was washed with brine (20 ml), dried over magnesium sulfate, and evaporated to dryness. The residue was purified on a silica gel (20 cc) eluting with 1˜10% methanol in chloroform to give (3S)-3-(propylamino)hexadecanamide (240 mg).

NMR (CDCl₃, δ): 7.52 (1H, br s), 5.61 (1H, br s), 3.05 (1H, m), 2.34-2.88 (5H, m), 1.49-1.73 (4H, m), 1.16-1.45 (22H, m), 0.99 (3H, t, J=7 Hz), 0.88 (3H, m)

### EXAMPLE 36

(3S)-3-[N-Propyl-{(2S)-5-benzyloxycarbonyl-2-(tert-butoxycarbonylamino)pentanoyl}amino]hexadecanamide was obtained according to a similar manner to that of Example 1.

Preparation 48

(3S)-3-[N-Propyl-{(2S)-2-amino-5-benzyloxycarbonylpentanoyl}amino]hexadecanamide was obtained according to a similar manner to that of Preparation 35.

### EXAMPLE 37

(3S)-3-[N-Propyl-{(2S)-5-benzyloxycarbonyl-2-(2-quinolylcarbonylamino)pentanoyl}amino]hexadecanamide was obtained according to a similar manner to that of Example 6.

### EXAMPLE 38

(3S)-3-[N-Propyl-{(2S)-5-carboxy-2-(2-quinolylcarbonylamino)pentanoyl}amino]hexadecanamide was obtained according to a similar manner to that of Example 21.

NMR (CDCl₃, δ): 8.83 (1H, d, J=9 Hz), 8.12-8.34 (3H, m), 7.86 (1H, d, J=8 Hz), 7.76 (1H, t, J=7 Hz), 7.61 (1H, t, J=7 Hz), 6.52 (2H, br s), 5.10 (2H, m), 3.08-3.50 (2H, m), 2.33-2.72 (4H, m), 1.47-2.16 (8H, m), 1.05-1.40 (22H, m), 1.00 (3H, t, J=7 Hz), 0.87 (3H, t, J=7 Hz)

The following compounds (Preparations 49 and 50) were obtained according to a similar manner to that of Preparation 1.

Preparation 49

Methyl 4-(4-biphenylyl)-3-oxobutanoate

mp: 80-82° C.

Preparation 50

Methyl 3-oxo-10-phenyldecanoate

NMR (CDCl₃, δ): 7.1-7.25 (5H, m), 3.74 (3H, s), 3.44 (2H, s), 2.5-2.7 (4H, m), 1.5-1.7 (4H, m), 1.2-1.4 (6H, m)

FAB-MS: 277 [M+H]

The following compounds (Preparations 51 to 60) were obtained according to a similar manner to that of Preparation 3.

Preparation 51

Methyl (3R)-4-(4-n-heptyl)phenyl-3-hydroxybutanoate

NMR (CDCl₃, δ): 7.12 (4H, s), 4.25 (1H, m), 3.70 (3H, s), 2.7-2.9 (3H, m), 2.4-2.6 (4H, m), 1.55-1.65 (2H, m), 1.2-1.4 (8H, m), 0.88 (3H, t, J=7 Hz)

Preparation 52

Methyl (3R)-4-(4-n-butyl)phenyl-3-hydroxybutanoate

NMR (CDCl₃, δ): 7.1 (4H, s), 4.24 (1H, m), 3.70 (3H, s), 2.7-2.85 (3H, m), 2.4-2.6 (4H, m), 1.5-1.65 (2H, m), 1.25-1.45 (2H, m), 0.90 (3H, t, J=7 Hz)

Preparation 53

Methyl (3R)-4-(4-methylphenyl)-3-hydroxybutanoate

NMR (CDCl₃, δ): 7.1 (4H, s), 4.22 (1H, m), 3.68 (3H, s), 2.76 (2H, ABX), 2.48 (2H, ABX), 2.32 (3H, s)

Preparation 54

Methyl (3S)-3-hydroxy-5-(2-naphthyl)pentanoate

NMR (CDCl₃, δ): 7.74-7.83 (3H, m), 7.64 (1H, s), 7.38-7.50 (2H, m), 7.35 (1H, d, J=8 Hz), 4.06 (1H, m), 3.71 (3H, s), 2.82-3.06 (3H, m), 2.42-2.60 (2H, m), 1.77-2.03 (2H, m)

Preparation 55

Methyl (3S)-5-(n-decyloxy)-3-hydroxypentanoate

NMR (CDCl₃, δ): 4.24 (1H, m), 3.71 (3H, s), 3.56-3.68 (2H, m), 3.53 (1H, d, J=3 Hz), 3.42 (2H, t, J=7 Hz), 2.48-2.54 (2H, m), 1.69-1.86 (2H, m), 1.50-1.61 (2H, m), 1.18-1.38 (14H, m), 0.87 (3H, t, J=7 Hz)

Preparation 56

Methyl (3S)-4-(4-biphenylyl)-3-hydroxybutanoate

Preparation 57

Methyl (3R)-3-hydroxydodecanoate

NMR (CDCl₃, δ): 4.0 (1H, m), 3.8 (3H, s), 2.82 (1H, d, J=3 Hz), 2.44 (2H, ABX), 1.2-1.55 (14H, m), 0.88 (3H, t, J=7 Hz)

Preparation 58

Methyl (3R)-3-hydroxynonanoate

NMR (CDCl₃, δ): 4.0 (1H, m), 3.72 (3H, s), 2.85 (1H, d, J=2 Hz), 2.45 (2H, ABX), 1.2-1.6 (10H, m), 0.86 (3H, t, J=7 Hz)

Preparation 59

Methyl (3R).-3-hydroxyheptanoate

NMR (CDCl₃, δ): 4.0 (1H, m), 3.7 (3H, s), 2.82 (1H, d, J=3 Hz), 2.44 (2H, ABX), 1.2-1.55 (6H, m), 0.88 (3H, t, J=7 Hz)

Preparation 60

Methyl (3R)-4-(6-ethyl-2-naphthyl)-3-hydroxybutanoate

NMR (CDCl₃, δ): 7.74-7.84 (3H, m), 7.66 (1H, s), 7.4-7.5 (2H, m), 7.34 (1H, d, j=8 Hz), 4.36 (1H, m), 3.7 (2H, ABX), 2.87 (1H, d, J=5 Hz), 2.52 (2H, ABX)

ESI-MS: 245 [M+H]

The following compounds (Preparations 61 to 72) were obtained according to a similar manner to that of Preparation 7.

Preparation 61

(3R)-4-(4-n-Heptyl)phenyl-3-hydroxybutanamide

NMR (CDCl₃, δ): 7.12 (4H, s), 5.88 (1H, br s), 5.46 (1H, br s), 4.22 (1H, m), 3.26 (1H, d, J=3 Hz), 2.7-2.9 (2H, m), 2.54-2.62 (2H, m), 2.38 (2H, ABX), 2.54-2.66 (2H, m), 1.2-1.4 (8H, m), 0.88 (3H, t, J=7 Hz)

Preparation 62

(3R)-4-(4-n-Butyl)phenyl-3-hydroxybutanamide

NMR (DMSO-d₆, δ): 7.26 (1H, br s), 7.06 (4H, s), 6.78 (1H, br s), 4.76 (1H, d, J=5 Hz), 4.02 (1H, m), 2.60 (2H, d, J=7 Hz), 2.5 (2H, m), 2.10 (2H, d, J=7 Hz), 1.45-1.6 (2H, m), 1.2-1.35 (2H, m), 0.88 (3H, t, J=7 Hz)

Preparation 63

(3R)-4-(4-Methylphenyl)-3-hydroxybutanamide

NMR (DMSO-d₆, δ): 7.26 (1H, br s), 7.06 (4H, s), 6.78 (1H, br s), 4.76 (1H, d, J=5 Hz), 4.00 (1H, m), 2.60 (2H, d, J=7 Hz), 2.24 (3H, s), 2.10 (2H, d, J=7 Hz)

Preparation 64

(3R)-3-Hydroxy-4-(2-naphthyl)butanamide

NMR (DMSO-d₆, δ): 7.78-7.90 (3H, m), 7.70 (1H, s), 7.34-7.52 (3H, m), 7.30 (1H, br s), 6.82 (1H, br s), 4.90 (1H, d, J=5.0 Hz), 4.14 (1H, m), 2.74-2.90 (2H, m), 2.15 (2H, d, J=5.0 Hz)

Preparation 65

(3S)-3-Hydroxy-5-(2-naphthyl)pentanamide

NMR (DMSO-d₆, δ): 7.80-7.90 (3H, m), 7.69 (1H, s), 7.36-7.52 (3H, m), 7.28 (1H, s), 6.80 (1H, br s), 4.79 (1H, d, J=5 Hz), 3.85 (1H, m), 2.68-2.96 (2H, m), 2.21 (2H, d, J=6 Hz), 1.60-1.84 (2H, m)

Preparation 66

(3S)-5-(n-Decyloxy)-3-hydroxypentanamide

Rf=0.16 (2% methanol in chloroform)

Preparation 67

(3S)-3-Hydroxy-10-phenyldecanamide

NMR (CDCl₃, δ): 7.1-7.35 (5H, m), 5.80 (1H, br s), 5.50 (1H, br s), 3.98 (1H, m), 3.30 (1H, d, J=3 Hz), 2.5-2.65 (2H, m), 2.2-2.45 (2H, m), 1.2-1.7 (10H, m)

FAB-MS: 264 [M+H]

Preparation 68

(3S)-4-(4-Biphenylyl)-3-hydroxybutanamide

NMR (DMSO-d₆, δ): 7.25-7.7 (10H, m), 6.85 (1H, br s), 4.86 (1H, d, J=7.5 Hz), 4.10 (1H, m), 2.52 (2H, s), 2.15 (2H, d, J=8.0 Hz)

FAB-MS: 256 [M+H]

Preparation 69

(3R)-3-Hydroxydodecanamide

NMR (CDCl₃, δ): 5.85 (1H, br s), 5.52 (1H, br s), 4.0 (1H, m), 3.3 (1H, d, J=3 Hz), 2.4 (2H, ABX), 1.2-1.6 (16H, m), 0.90 (3H, t, J=7 Hz)

Preparation 70

(3R)-3-Hydroxynonanamide

NMR (CDCl₃, δ): 5.85 (1H, br s), 5.52 (1H, br s), 4.0 (1H, m), 3.3 (1H, br s), 2.38 (2H, ABX), 1.2-1.7 (10H, m), 0.88 (3H, t, J=7 Hz)

Preparation 71

(3R)-3-Hydroxyheptanamide

NMR (CDCl₃, δ): 5.85 (1H, br s), 5.6 (1H, br s), 4.0 (1H, m), 3.32 (1H, br s), 2.38 (2H, ABX), 1.2-1.8 (8H, m), 0.88 (3H, t, J=7 Hz)

Preparation 72

(3R)-4-(6-Ethyl-2-naphthyl)-3-hydroxybutanamide

NMR (DMSO-d₆, δ): 7.76 (2H, dd, J=8, 4 Hz), 7.64 (2H, s), 7.34 (2H, d, J=8 Hz), 7.28 (2H, br s), 6.80 (1H, br s), 4.86 (1H, d, J=4 Hz), 4.14 (1H, m), 2.8 (2H, d, J=7.5 Hz), 2.74 (2H, q, J=7.5 Hz), 2.16 (2H, d, J=7.5 Hz), 1.26 (3H, t, J=7.5 Hz)

Preparation 73

[4-(n-Butyl)phenyl]acetic acid was obtained according to

a similar manner to that of Preparation 10.

NMR (CDCl₃, δ): 7.10-7.22 (4H, m), 3.60 (2H, s), 2.58 (2H, dd, J=8, 7 Hz), 1.41-1.64 (2H, m), 1.27-1.42 (2H, m), 0.92 (3H, t, J=7 Hz)

The following compounds (Preparations 74 to 77) were obtained according to a similar manner to that of Preparation 14.

Preparation 74

Methyl 4-(4-n-butyl)phenyl-3-oxobutanoate NMR (CDCl₃, δ): 7.05-7.15 (4H, m), 3.76 (2H, s), 3.70 (3H, s), 3.42 (2H, s), 2.58 (2H, t, J=7 Hz), 1.5-1.65 (2H, m), 1.25-1.4 (2H, m), 0.92 (3H, t, J=7 Hz)

Preparation 75

Methyl 4-(4-methylphenyl)-3-oxobutanoate

NMR (CDCl₃, δ): 7.0-7.15 (4H, m), 3.74 (2H, s), 3.68 (3H, s), 3.42 (2H, s), 2.3 (3H, s)

Preparation 76

Methyl 5-(2-naphthyl)-3-oxopentanoate

NMR (CDCl₃, δ): 7.74-7.83 (3H, m), 7.63 (1H, br s), 7.38-7.49 (2H, m), 7.32 (1H, d, J=8 Hz), 3.72 (3H, s), 3.46 (2H, s), 3.09 (2H, t, J=7 Hz), 2.97 (2H, t, J=7 Hz)

Preparation 77

Methyl 5-(n-decyloxy)-3-oxopentanoate

NMR (CDCl₃, δ): 3.74 (3H, s), 3.67 (2H, t, J=7 Hz), 3.51 (2H, s), 3.39 (2H, t, J=7 Hz), 2.78 (2H, t, J=7 Hz), 1.46-1.58 (2H, m), 1.18-1.36 (14H, m), 0.87 (3H, t, J=7 Hz)

The following compounds (Preparations 78 to 85) were obtained according to a similar manner to that of Preparation 17.

Preparation 78

(3R)-4-(4-n-Heptyl)phenyl-3-methanesulfonyloxybutanamide

NMR (CDCl₃, δ): 7.05-7.2 (4H, m), 5.6 (1H, br s), 5.4 (1H, br s), 5.12 (1H, m), 3.08 (2H, d, J=7 Hz), 2.70 (3H, s), 2.52-2.62 (4H, m), 1.5-1.65 (2H, m), 1.2-1.35 (8H, m), 0.88 (3H, t, J=7 Hz)

Preparation 79

(3R)-4-(4-n-Butyl)phenyl-3-methanesulfonyloxybutanamide

NMR (CDCl₃, δ): 7.05-7.2 (4H, m), 5.64 (1H, br s), 5.54 (1H, br s), 5.14 (1H, m), 3.08 (2H, d, J=7 Hz), 2.70 (3H, s), 2.54-2.64 (4H, m), 1.5-1.65 (2H, m), 1.25-1.4 (2H, m), 0.92 (3H, t, J=7 Hz)

Preparation 80

(3R)-4-(4-Methylphenyl)-3-methanesulfonyloxybutanamide

NMR (CDCl₃, δ): 7.05-7.15 (4H, m), 5.60 (1H, br s), 5.44 (1H, br s), 5.14 (1H, m), 3.08 (2H, d, J=7 Hz), 2.70 (3H, s), 2.64 (2H, d, J=7 Hz), 2.30 (3H, s)

Preparation 81

(3R)-3-Methanesulfonyloxy-4-(2-naphthyl)butanamide

Preparation 82

(3R)-3-Methanesulfonyloxydodecanamide

NMR (CDCl₃, δ): 5.72 (1H, br s), 5.52 (1H, br s), 5.05 (1H, m), 3.04 (3H, s), 2.62 (2H, d, J=7 Hz), 1.83 (2H, m), 1.2-1.5 (14H, m), 0.90 (3H, t, J=7 Hz)

Preparation 83

(3R)-3-Methanesulfonyloxynonanamide

NMR (CDCl₃, δ): 5.84 (1H, br s), 5.76 (1H, br s), 5.02 (1H, m), 3.02 (3H, s), 2.6 (2H, d, J=7 Hz), 1.7-1.9 (2H, m), 1.2-1.45 (8H, m), 0.88 (3H, t, J=7 Hz)

Preparation 84

(3R)-3-Methanesulfonyloxyheptanamide

NMR (CDCl₃, δ): 5.9 (1H, br s), 5.8 (1H, br s), 5.04 (1H, m), 3.04 (3H, s), 2.62 (2H, d, J=7 Hz), 1.7-1.9 (2H, m), 1.2-1.4 (4H, m), 0.90 (3H, t, J=7 Hz)

Preparation 85

(3R)-4-(6-Ethyl-2-naphthyl)-3-methanesulfonyloxybutanamide

NMR (DMSO-d₆, δ): 7.8 (2H, d, J=8, 4 Hz), 7.7 (2H, d, J=8 Hz), 7.48 (1H, br s), 7.28 (2H, dd, J=8, 3 Hz), 7.04 (1H, br s), 5.22 (1H, m), 3.2 (2H, ABX), 2.95 (3H, s), 2.76 (2H, q, J=7 Hz), 2.46 (2H, d, J=7 Hz), 1.26 (3H, t, J=7 Hz)

The following compounds (Preparations 86 to 93) were obtained according to a similar manner to that of Preparation 18.

Preparation 86

(3S)-3-Azido-4-(4-n-heptylphenyl)butanamide

Preparation 87

(3S)-3-Azido-4-(4-n-butylphenyl)butanamide

Preparation 88

(3S)-3-Azido-4-(4-methylphenyl)butanamide

Preparation 89

(3S)-3-Azido-4-(2-naphthyl)butanamide

Preparation 90

(3S)-3-Azidododecanamide

NMR (CDCl₃, δ): 5.66 (1H, br s), 5.55 (1H, br s), 3.84 (1H, m), 2.3-2.5 (2H, m), 1.58 (2H, t, J=7 Hz), 1.2-1.5 (14H, m), 0.88 (3H, t, J=7 Hz)

Preparation 91

(3S)-3-Azidononanamide

NMR (CDCl₃, δ): 5.65 (1H, br s), 5.55 (1H, br s), 3.86 (1H, m), 2.4 (2H, ABX), 1.2-1.65 (10H, m), 0.88 (3H, t, J=7 Hz)

Preparation 92

(3S)-3-Azidoheptanamide

NMR (CDCl₃, δ): 5.65 (1H, br s), 5.55 (1H, br s), 3.82 (1H, m), 2.4 (2H, ABX), 1.2-1.7 (6H, m), 0.9 (3H, t, J=7 Hz)

Preparation 93

(3S)-3-Azido-4-(6-ethyl-2-naphthyl)butanamide

The following compounds (Preparations 94 to 101) were obtained according to a similar manner to that of Preparation 19.

Preparation 94

(3S)-3-Amino-4-(4-n-heptylphenyl)butanamide hydrochloride

NMR (DMSO-d₆, δ): 8.04 (3H, br s), 7.62 (1H, br s), 7.05-7.2 (5H, m), 3.56 (1H, m), 2.96 (1H, dd, J=12, 5 Hz), 2.74 (1H, dd, J=12, 8 Hz), 2.5-2.6 (2H, m), 2.35 (2H, d, J=7 Hz), 1.5-1.65 (2H, m), 1.2-1.35 (8H, m), 0.86 (3H, t, J=7 Hz)

Preoaration 95

(3S)-3-Amino-4-(4-n-butylphenyl)butanamide hydrochloride

NMR (DMSO-d₆, δ): 8.06 (3H, br s), 7.62 (1H, br s), 7.1-7.2 (5H, m), 3.58 (1H, m), 2.96 (1H, dd, J=12, 8 Hz), 2.74 (1H, dd, J=12, 5 Hz), 2.54 (2H, t, J=7 Hz), 2.36 (2H, d, J=7 Hz), 1.48-1.62 (2H, m), 1.22-1.38 (2H, m), 0.90 (3H, t, J=7 Hz)

Preparation 96

(3S)-3-Amino-4-(4-methylphenyl)butanamide hydrochloride

NMR (DMSO-d₆, δ): 8.06 (3H, br s), 7.62 (1H, br s), 7.05-7.2 (5H, m), 3.56 (1H, m), 2.96 (1H, dd, J=12, 5 Hz), 2.74 (1H, dd, J=12, 8 Hz), 2.74 (2H, t, J=7 Hz), 2.34 (2H, d, J=7 Hz), 2.28 (3H, s)

Preparation 97

(3S)-3-Amino-4-(2-naphthyl)butanamide

NMR (CD₃OD-CDCl₃, 5): 7.75-7.87 (3H, m), 7.64 (1H, s), 7.42-7.54 (2H, m), 7.32 (1H, d, J=8 Hz), 7.11 (0.25H, br s), 5.62 (0.25H, br s), 3.54 (1H, m), 2.98 (1H, dd, J=14, 6 Hz), 2.76 (1H, dd, J=14, 8 Hz), 2.46 (1H, dd, J=15, 3 Hz), 2.26 (1H, dd, J=15, 8 Hz)

Preparation 98

(3S)-3-Aminododecanamide hydrochloride

NMR (DMSO-d₆, δ): 8.04 (3H, br s), 7.66 (1H, br s), 7.12 (1H, br s), 3.32 (1H, m), 2.42 (2H, d, J=7 Hz), 1.38-1.6 (2H, m), 1.15-1.38 (14H, m), 0.84 (3H, t, J=7 Hz)

Preparation 99

(3S)-3-Aminononanamide hydrochloride

NMR (DMSO-d₆, δ): 8.08 (3H, br s), 7.68 (1H, br s), 7.12 (1H, br s), 3.32 (1H, m), 2.44 (2H, d, J=7 Hz), 1.4-1.65 (2H, m), 1.2-1.4 (8H, m), 0.86 (3H, t, J=7 Hz)

Preparation 100

(3S)-3-Aminoheptanamide

NMR (DMSO-d₆, δ): 7.36 (1H, br s), 6.72 (1H, br s), 2.94 (1H, m), 2.10 (1H, dd, J=12, 5 Hz), 1.96 (1H, dd, J=12, 8 Hz), 1.15-1.4 (6H, m), 0.84 (3H, t, J=7 Hz)

Preparation 101

(3S)-3-Amino-4-(6-ethyl-2-naphthyl)butanamide

NMR (DMSO-d₆, δ): 7.8 (2H, m), 7.7 (2H, d, J=7 Hz), 7.62 (1H, br s), 7.36 (2H, m), 7.1 (1H, br s), 3.7 (1H, m), 3.16 (1H, dd, J=12, 5 Hz), 2.95 (1H, dd, J=12, 7 Hz), 2.76 (2H, q, J=7 Hz), 2.4 (2H, d, J=7 Hz), 1.26 (3H, t, J=7 Hz)

The following compounds (Preparations 102 to 127) were obtained according to a similar manner to that of Preparation 47.

Preparation 102

(3S)-4-(2-Naphthyl)-3-(n-propylamino)butanamide

NMR (CDCl₃, δ): 8.20 (1H, br s), 7.76-7.86 (3H, m), 7.60 (1H, s), 7.4-7.5 (2H, m), 7.30 (1H, d, J=8 Hz), 5.34 (1H, br s), 3.26 (1H, m), 2.98 (2H, dd, J=7, 2 Hz), 2.65 (2H, t, J=7 Hz), 2.52 (1H, dd, J=12, 3 Hz), 2.26 (1H, dd, J=12, 5 Hz), 1.4-1.6 (2H, m), 0.88 (3H, t, J=7 Hz)

ESI-MS: 271 [M+H]

Preparation 103

(3S)-3-(n-Butyl)amino-4-(4-n-heptylphenyl)butanamide

NMR (CDCl₃, δ): 8.26 (1H, br s), 7.0-7.2 (4H, m), 5.34 (1H, br s), 3.12 (1H, m), 2.76 (2H, d, J=7 Hz), 2.54-2.7 (4H, m), 2.48 (1H, dd, J=12, 3 Hz), 2.20 (1H, dd, J=12, 5 Hz), 1.5-1.7 (6H, m), 1.2-1.5 (8H, m), 0.85-0.95 (6H, m)

Preparation 104

(3S)-3-(n-Butyl)amino-4-(4-n-butylphenyl)butanamide

NMR (CDCl₃, δ): 8.08 (1H, br s), 7.0-7.2 (4H, m), 5.38 (1H, br s), 3.15 (1H, m), 2.0-2.9 (9H, m), 1.25-1.65 (8H, m), 0.85-0.95 (6H, m)

Preparation 105

(3S)-4-(4-n-Butyl)phenyl-3-(n-propylamino)butanamide NMR (CDCl₃, δ): 7.82 (1H, br s), 7.0-7.2 (4H, m), 5.46 (1H, br s), 3.20 (1H, m), 2.25-2.95 (9H, m), 1.25-1.65 (6H, m), 0.85-0.95 (6H, m)

Preparation 106

(3S)-4-(4-Methylphenyl)-3-(n-propylamino)butanamide

NMR (CDCl₃, δ): 8.22 (1H, br s), 7.0-7.2 (4H, m), 5.34 (1H, br s), 3.12 (1H, m), 2.76 (2H, dd, J=7, 3Hz), 2.62 (2H, dt, J=7, 2 Hz), 2.46 (1H, dd, J=12, 3 Hz), 2.32 (3H, s), 2.20 (1H, dd, J=12, 7 Hz), 1.4-1.55 (2H, m), 0.88 (3H, t, J=7 Hz)

Preparation 107

(3S)-3-(n-Butyl)amino-4-(2-naphthyl)butanamide

NMR (CDCl₃, δ): 8.20 (1H, br s), 7.76-7.86 (3H, m), 7.60 (1H, s), 7.4-7.5 (2H, m), 7.30 (1H, d, J=8 Hz), 5.34 (1H, br s), 3.24 (1H, m), 2.98 (2H, dd, J=7, 2 Hz), 2.65 (2H, t, J=7 Hz), 2.52 (1H, dd, J=12, 3 Hz), 2.24 (1H, dd, J=12, 5 Hz), 1.2-1.5 (4H, m), 0.88 (3H, t, J=7 Hz)

Preparation 108

(3S)-3-Ethylamino-4-(2-naphthyl)butanamide

Preparation 109

(3S)-3-Isopentylaminohexadecanamide

NMR (CDCl₃, δ): 8.12 (1H, br s), 5.38 (1H, br s), 2.86 (1H, m), 2.1-2.25 (2H, m), 2.50 (1H, dd, J=12, 3 Hz), 2.26 (1H, dd, J=12, 8 Hz), 1.2-1.7 (27H, m), 0.8-0.9 (9H, m)

ESI-MS: 341 [M+H]

Preparation 110

(3S)-3-Isobutylaminohexadecanamide

NMR (CDCl₃, δ): 8.22 (1H, br s), 5.32 (1H, br s), 2.84 (1H, m), 2.35-2.6 (3H, m), 2.22 (1H, dd, J=12, 8 Hz), 1.2-1.8 (25H, m), 0.92-0.98 (6H, m), 0.86 (3H, t, J=7 Hz)

ESI-MS: 327 [M+H]

Preparation 111

(3S)-4-(2-Naphthyl)-3-(n-pentylamino)butanamide NMR (CDCl₃, δ): 8.20 (1H, br s), 7.76-7.87 (3H, m), 7.62 (1H, s), 7.42-7.52 (1H, m), 7.31 (1H, dd, J=8, 3 Hz), 5.35 (1H, br s), 3.25 (1H, m), 2.98 (2H, d, J=7 Hz), 2.67 (2H, t, J=7 Hz), 2.53 (1H, dd, J=16, 3 Hz), 2.24 (1H, dd, J=16, 7 Hz), 1.36-1.50 (2H, m), 1.18-1.34 (4H, m), 0.84 (3H, t, J=7 Hz)

ESI-MS: 299 [M+H]

Preparation 112

(3S)-3-Ethylaminohexadecanamide

Preparation 113

(3S)-3-(n-Butylamino)hexadecanamide

NMR (CDCl₃, δ): 7.48 (1H, s), 5.65 (1H, s), 3.09 (1H, m), 2.87 (1H, m), 2.43-2.79 (3H, m), 1.08-1.76 (28H, m), 0.96 (3H, t, J=7 Hz), 0.88 (3H, t, J=7 Hz)

ESI-MS: 327 [M+H]

Preparation 114

(3S)-3-Phenethylamino)hexadecanamide

NMR (CDCl₃, δ): 7.95 (1H, br s), 7.15-7.35 (5H, m), 5.08 (1H, br s), 2.74-3.02 (4H, m), 2.38 (1H, dd, J=16, 3 Hz), 2.24 (1H, dd, J=16, 8 Hz), 1.08-1.69 (24H, m), 0.88 (3H, t, J=7 Hz)

ESI-MS: 375 [M+H]

Preparation 115

(3S)-3-(2-Pyridylmethylamino)hexadecanamide

NMR (CDCl₃, δ): 8.56 (1H, m), 8.12 (1H, br s), 7.65 (1H, dd, J=8, 7 Hz), 7.16-7.29 (2H, m), 5.31 (1H, br s), 3.95 (1H, s), 2.95 (1H, m), 2.52 (1H, dd, J=16, 4 Hz), 2.24 (1H, dd, J=16, 7 Hz), 1.16-1.68 (24H, m), 0.88 (3H, t, J=7 Hz)

ESI-MS: 362 [M+H]

Preparation 116

(3S)-3-Benzylaminohexadecanamide

NMR (CDCl₃, δ): 8.09 (1H, br s), 7.23-7.39 (5H, m), 5.28 (1H, br s), 3.83 (1H, d, J=14 Hz), 3.76 (1H, d, J=14 Hz), 2.95 (1H, m), 2.51 (1H, dd, J=13, 4 Hz), 2.24 (1H, dd, J=13, 8 Hz), 1.17-1.67 (24H, m), 0.88 (3H, t, J=7 Hz)

ESI-MS: 361 [M+H]

Preparation 117

(3S)-3-(n-Pentylamino)dodecanamide

Preparation 118

(3S)-3-(n-Propylamino)dodecanamide

Preparation 119

(3S)-3-(n-Pentylamino)nonanamide

Preparation 120

(3S)-3-(n-Butylamino)nonanamide

Preparation 121

(3S)-3-(n-Propylamino)nonanamide

Preparation 122

(3S)-3-Ethylaminononanamide

ESI-MS: 201 [M+H]

Preparation 123

(3S)-3-(n-Propylamino)heptanamide

Preparation 124

(3S)-3-(n-Butylamino)heptanamide

ESI-MS: 201 [M+H]

Preparation 125

(3S)-3-(n-Propyl)amino-4-(6-ethyl-2-naphthyl)butanamide

Preparation 126

(3S)-3-(n-Butyl)amino-4-(6-ethyl-2-naphthyl)butanamide

Preparation 127

(3S)-3-(n-Pentylamino)hexadecanamide

NMR (CDCl₃, δ): 7.80 (1H, br s), 5.52 (1H, br s), 3.00 (1H, m), 2.63-2.86 (2H, m), 2.57 (1H, dd, J=13, 8 Hz), 2.38 (1H, dd, J=13, 8 Hz), 1.18-1.68 (30H, m), 0.84-0.98 (6H, m)

ESI-MS: 341 [M+H]

The following compounds (Preparations 128 to 157) were obtained according to a similar manner to that of Preparation 35.

Preparation 128

(3S)-3-[N-(n-Butyl)-{(2S)-2-amino-5-methoxycarbonylpentanoyl}amino]-4-(4-n-heptylphenyl)butanamide hydrochloride

ESI-MS: 490 [M+H]

Preparation 129

(3S)-3-[N-(n-Propyl)-{(2S)-2-amino-4-benzyloxycarbonylbutanoyl}amino]-4-(2-naphthyl)butanamide hydrochloride

ESI-MS 490 [M(free)+H]

Preparation 130

(3S)-3-[N-(n-Butyl)-{(2S)-2-amino-5-benzyloxycarbonyl-pentanoyl}amino]-4-(4-n-butylphenyl)butanamide hydrochloride

ESI-MS: 524 [M(free)+H]

Preparation 131

(3S)-3-[N-(n-Propyl)-{(2S)-2-amino-5-benzyloxycarbonylpentanoyl}amino]-4-(4-n-butylphenyl)butanamide hydrochloride

ESI-MS: 510 [M(free)+H]

Preparation 132

(3S)-3-[N-(n-Propyl)-{(2S)-2-amino-5-benzyloxycarbonylpentanoyl}amino]-4-(4-methylphenyl)butanamide hydrochloride

ESI-MS 468 [M(free)+H]

Preparation 133

(3S)-3-[N-(n-Butyl)-{(2S)-2-amino-5-benzyloxycarbonylpentanoyl}amino]-4-(2-naphthyl)butanamide hydrochloride

ESI-MS: 518 [M(free)+H]

Preparation 134

(3S)-3-[N-Ethyl-{(2S)-2-amino-5-benzyloxycarbonylpentanoyl}amino]-4-(2-naphthyl)butanamide hydrochloride

ESI-MS: 490 [M(free)+H]

Preparation 135

(3S)-3-[N-Isopropyl-{(2S)-2-amino-5-benzyloxycarbonylpentanoyl}amino]hexadecanamide hydrochloride

ESI-MS: 574 [M(free)+H]

Preparation 136

(3S)-3-[N-Isobutyl-{(2S)-2-amino-5-benzyloxycarbonylpentanoyl}amino]hexadecanamide hydrochloride

ESI-MS: 560 [M(free)+H]

Preparation 137

(3S)-3-[N-(n-Pentyl)-{(2S)-2-amino-5-benzyloxycarbonylpentanoyl}amino]-4-(2-naphthyl)butanamide hydrochloride

ESI-MS 532 [M(free)+H]

Preparation 138

(3S)-3-[N-(n-Propyl)-{(2S)-2-amino-5-benzyloxycarbonylpentanoyl}amino]-4-(2-naphthyl)butanamide hydrochloride

ESI-MS: 504 [M(free)+H]

Preparation 139

(3S)-3-[N-Ethyl-{(2S)-2-amino-5-benzyloxycarbonylpentanoyl}amino]hexadecanamide hydrochloride

ESI-MS: 532 [M(free)+H]

Preparation 140

(3S)-3-[N-(n-Propyl)-{(2S)-2-amino-5-benzyloxycarbonylpentanoyl}amino]hexadecanamide hydrochloride

ESI-MS 560 [M+H]

Preparation 141

(3S)-3-[N-Phenethyl-{(2S)-2-amino-5-benzyloxycarbonylpentanoyl}amino]hexadecanamide hydrochloride

ESI-MS 608 [M+H]

Preparation 142

(3S)-3-[N-(n-Pentyl)-{(2S)-2-amino-5-benzyloxycarbonylpentanoyl}amino]hexadecanamide hydrochloride

ESI-MS: 574 [M(free)+H]

Preparation 143

(3S)-3-[N-Benzyl-{(2S)-2-amino-5-benzyloxycarbonylpentanoyl}amino]hexadecanamide hydrochloride

ESI-MS: 594 [M(free)+H]

Preparation 144

(3S)-3-[N-(2-Pyridylmethyl)-{(2S)-2-amino-5-benzyloxycarbonylpentanoyl}amino]hexadecanamide dihydrochloride

ESI-MS 595 [M(free)+H]

Preparation 145

(3S)-3-r[(2S)-2-Amino-5-benzyloxycarbonylpentanoyl]-oxy-5-(2-naphthyl)pentanamide hydrochloride

NMR (DMSO-d₆, δ): 8.57 (2H, br s), 7.80-7.92 (3H, m), 7.73 (1H, s), 7.30-7.45 (8H, m), 6.92 (1H, br s), 5.30 (1H, m), 5.09 (2H, s), 4.04 (1H, m), 2.72-2.92 (2H, m), 2.23-2.50 (4H, m), 1.52-2.07 (6H, m)

Preparation 146

(3S)-3-[(2S)-2-Amino-5-benzyloxycarbonylpentanoyl]-oxy-10-phenyldecanamide hydrochloride

NMR (CDCl₃, δ): 8.2 (2H, br), 7.1-7.4 (11H, m), 6.75 (1H, s), 5.3 (1H, m), 5.08 (2H, s), 4.02 (1H, t, J=5.0 Hz), 2.54 (3H, m), 2.40 (3H, m), 1.5-2.15 (8H, m), 1.2-1.35 (8H, m)

Preparation 147

3-[N-Methyl-{(2S)-2-amino-5-benzyloxycarbonylpentanoyl}-amino]propanamide hydrochloride

Preparation 148

(3S)-3-[N-(n-Pentyl)-{(2S)-2-amino-5-benzyloxycarbonylpentanoyl}amino]dodecanamide hydrochloride

Preparation 149

(3S)-3-[N-(n-Propyl)-{(2S)-2-amino-5-methoxycarbonylpentanoyl}amino]dodecanamide hydrochloride

ESI-MS: 414 [M(free)+H]

Preparation 150

(3S)-3-[N-(n-Pentyl)-{(2S)-2-amino-5-benzyloxycarbonylpentanoyl}amino]nonanamide hydrochloride

Preparation 151

(3S)-3-[N-(n-Butyl)-{(2S)-2-amino-5-benzyloxycarbonylpentanoyl}amino]nonanamide hydrochloride

Preparation 152

(3S)-3-[N-(n-Propyl)-{(2S)-2-amino-5-benzyloxycarbonylpentanoyl}amino]nonanamide hydrochloride

Preparation 153

(3S)-3-[N-Ethyl-{(2S)-2-amino-5-methoxycarbonylpentanoyl}amino]nonanamide hydrochloride

ESI-MS 358 [M(free)+H]

Preparation 154

(3S)-3-[N-(n-Propyl)-{(2S)-2-amino-5-benzyloxycarbonylpentanoyl}amino] heptanamide hydrochloride

Preparation 155

(3S)-3-[N-(n-Butyl)-{(2S)-2-amino-5-methoxycarbonylpentanoyl}amino]heptanamide hydrochloride

ESI-MS 358 [M(free)+H]

Preparation 156

(3S)-3-[N-(n-Propyl)-{(2S)-2-amino-5-benzyloxycarbonylpentanoyl}amino]-4-(6-ethyl-2-naphthyl)butanamide hydrochloride

Preparation 157

(3S)-3-[N-(n-Butyl)-{(2S)-2-amino-5-benzyloxycarbonylpentanoyl}amino]-4-(6-ethyl-2-naphthyl)butanamide hydrochloride

The following compounds (Examples 39 to 43) were obtained according to a similar manner to that of Example 1.

### EXAMPLE 39

(3S)-3-[(2S)-5-Benzyloxycarbonyl-2-(tert-butoxycarbonylamino)pentanoyl]oxy-10-phenyldecanamide

NMR (CDCl₃, δ): 7.1-7.4 (5H, m), 5.9 (1H, br s), 5.3 (1H, br s), 5.22 (1H, m), 5.1 (2H, s), 5.06 (1H, d, J=10.0 Hz), 4.22 (1H, m), 2.08 (2H, m), 2.3-2.5 (4H, m), 1.2-1.9 (30H, m)

FAB-MS: 619 [M+Na]

### EXAMPLE 40

(3S)-3-[(2S)-5-Benzyloxycarbonyl-2-(tert-butoxycarbonylamino)pentanoyl]oxy-4-(4-biphenylyl)butanamide

mp: 94-98° C.

### EXAMPLE 41

(3S)-3-[N-(n-Butyl)-{(2S)-5-benzyloxycarbonyl-2-(tert-butoxycarbonylamino)pentanoyl}amino]-4-(4-n-butylphenyl)butanamide

ESI-MS 624 [M+H]

### EXAMPLE 42

(3S)-3-[(2S)-5-Benzyloxycarbonyl-2-(tert-butoxycarbonylamino)pentanoyl]oxy-5-(2-naphthyl)pentanamide

NMR (CDCl₃, δ): 7.74-7.84 (3H, m), 7.63 (1H, s), 7.28-7.51 (8H, m), 5.93 (1H, br s), 5.33 (1H, m), 5.30 (1H, br s), 5.12 (2H, s), 4.97 (1H, d, J=7 Hz), 4.23 (1H, m), 2.84 (2H, t, J=7 Hz), 2.52 (2H, d, J=6 Hz), 2.33-2.42 (2H, m), 2.04-2.18 (2H, m), 1.60-1.90 (4H, m), 0.95 (9H, s)

### EXAMPLE 43

(3S)-3-[(2S)-5-Benzyloxycarbonyl-2-(tert-butoxycarbonylamino)pentanoyl]oxy-5-(n-decyloxy)pentanamide

NMR (CDCl₃, δ): 7.31-7.40 (5H, m), 6.02 (1H, br s), 5.36 (1H, m), 5.28 (1H, br s), 5.12 (2H, s), 5.06 (1H, d, J=8 Hz), 4.23 (1H, m), 3.34-3.57 (4H, m), 2.59 (1H, dd, J=15, 6 Hz), 2.51 (1H, dd, J=15, 7 Hz), 1.49-2.01 (8H, m), 1.44 (9H, s), 1.21-1.36 (14H, m), 0.88 (3H, t, J=7 Hz)

The following compounds (Examples 44 to 136) were obtained according to a similar manner to that of Example 6.

### EXAMPLE 44

(3S)-3-[(2S)-5-Benzyloxycarbonyl-2-[2-(methoxycarbonyl-methyl)benzoylamino]pentanoyl]oxy-4-(2-naphthyl)butanamide

NMR (CDCl₃, δ): 7.72-7.83 (3H, m), 7.65 (1H, s), 7.52 (1H, d, J=7 Hz), 7.22-7.47 (12H, m), 5.93 (1H, br s), 5.59 (1H, m), 5.25 (1H, br s), 5.08 (2H, s), 4.59 (1H, m), 3.93 (1H, d, J=15 Hz), 3.80 (1H, d, J=15 Hz), 3.67 (3H, s), 3.18 (2H, d, J=7 Hz), 2.43-2.59 (2H, m), 2.16-2.34 (2H, m), 1.48-1.83 (4H, m)

ESI-MS: 639 [M+H]

### EXAMPLE 45

(3S)-3-[(2S)-5-Benzyloxycarbonyl-2-[2-(benzyloxy-carbonylmethyl)benzoylamino]pentanoyl]oxy-4-(2-naphthyl)butanamide

NMR (CDCl₃, δ): 7.72-7.82 (3H, m), 7.66 (1H, s), 7.52 (1H, d, J=7 Hz), 7.22-7.47 (17H, m), 5.89 (1H, br s), 5.57 (1H, m), 5.22 (1H, br s), 5.12 (2H, s), 5.07 (2H, s), 4.57 (1H, m), 3.97 (1H, d, J=16 Hz), 3.86 (1H, d, J=16 Hz), 3.17 (2H, d, J=7 Hz), 2.42-2.57 (2H, m), 2.13-2.31 (2H, m), 1.48-1.79 (4H, m)

ESI-MS: 715 [M+H]

### EXAMPLE 46

(3S)-3-[(2S)-5-Benzyloxycarbonyl-2-[2-((2E)-2-methoxycarbonylvinyl)benzoylamino]pentanoyl]oxy-4-(2-naphthyl)butanamide

NMR (CDCl₃, δ): 7.98 (1H, d, J=16 Hz), 7.60-7.77 (5H, m), 7.29-7.53 (11H, m), 6.45 (1H, d, J=8 Hz), 6.34 (1H, d, J=16 Hz), 6.18 (1H, br s), 5.63 (1H, m), 5.37 (1H, br s), 5.09 (2H, s), 4.61 (1H, m), 3.75 (3H, s), 3.10-3.26 (2H, m), 2.54-2.69 (2H, m), 2.12-2.36 (2H, m), 1.46-1.86 (4H, m)

ESI-MS: 651 [M+H]

### EXAMPLE 47

(3S)-3-[(2S)-5-Benzyloxycarbonyl-2-(3-benzylnaphthalen-2-ylcarbonylamino)pentanoyl]oxy-4-(4-n-heptylphenyl)butanamide

NMR (CDCl₃, δ): 7.93 (1H, s), 7.75-7.88 (2H, m), 7.66 (1H, s), 7.46-7.58 (2H, m), 7.27-7.38 (5H, m), 7.06-7.24 (9H, m), 6.33 (1H, d, J=7 Hz), 5.75 (1H, br s), 5.46 (1H, m), 5.23 (1H, br s), 5.08 (2H, s), 4.53 (1H, m), 4.47 (1H, d, J=16 Hz), 4.28 (1H, d, J=16 Hz), 2.88-3.04 (2H, m), 2.47-2.56 (2H, m), 2.37-2.45 (2H, m), 2.22-2.32 (2H, m), 1.38-1.82 (6H, m), 1.15-1.34 (8H, m), 0.82-0.92 (3H, m)

ESI-MS: 755 [M+H]

### EXAMPLE 48

(3S)-3-[(2S)-5-Benzyloxycarbonyl-2-(2-quinolylcarbonylamino)pentanoyl]oxy-5-(2-naphthyl)pentanamide

NMR (CDCl₃, δ): 8.73 (1H, d, J=8 Hz), 8.33 (1H, d, J=8 Hz), 8.26 (1H, d, J=8 Hz), 8.18 (1H, d, J=8 Hz), 7.90 (1H, d, J=8 Hz), 7.61-7.83 (5H, m), 7.59 (1H, s), 7.25-7.46 (7H, m), 5.96 (1H, br s), 5.41 (1H, m), 5.29 (1H, br s), 5.12 (2H, s), 4.77 (1H, m), 2.86 (2H, t, J=8 Hz), 2.57 (2H, d, J=6 Hz), 2.45 (2H, d, J=7 Hz), 1.75-2.22 (6H, m)

### EXAMPLE 49

(3S)-3-[(2S)-5-Benzyloxycarbonyl-2-[1-(4-methylbenzyl)indol-2-ylcarbonylamino]pentanoyl]oxy-4-(2-naphthyl)butanamide

NMR (CDCl₃, δ): 7.67-7.78 (4H, m), 7.63 (1H, s), 7.27-7.45 (9H, m), 7.17 (1H, m), 6.86-7.05 (6H, m), 5.84 (1H, br s), 5.81 (1H, d, J=16 Hz), 5.68 (1H, d, J=16 Hz), 5.57 (1H, m), 5.16 (1H, br s), 5.11 (2H, s), 4.55 (1H, m), 3.08 (2H, m), 2.07-2.48 (4H, m), 2.25 (3H, s), 1.38-1.82 (4H, m)

ESI-MS: 710 [M+H]

### EXAMPLE 50

(3S)-3-[(2S)-5-Benzyloxycarbonyl-2-[1-(4-chlorobenzyl)-indol-3-ylcarbonylamino]pentanoyl]oxy-4-(2-naphthyl)butanamide

NMR (CDCl₃, δ): 8.07 (1H, m), 7.67-7.78 (4H, m), 7.53 (1H, s), 7.21-7.47 (13H, m), 7.07 (2H, d, J=8 Hz), 6.86 (1H, d, J=7 Hz), 6.07 (1H, br s), 5.62 (1H, m), 5.32 (1H, br s), 5.30 (2H, s), 5.08 (2H, s), 4.70 (1H, m), 3.16 (2H, d, j=7 Hz), 2.57 (1H, dd, J=15, 4 Hz), 2.48 (1H, dd, J=15, 7 Hz), 2.11-2.36 (2H, m), 1.45-1.90 (4H, m)

ESI-MS: 730 [M+H]

### EXAMPLE 51

(3S)-3-[(2S)-5-Benzyloxycarbonyl-2-[1-(4-methylbenzyl)indol-3-ylcarbonylamino]pentanoyl]oxy-4-(2-naphthyl)butanamide

NMR (CDCl₃, δ): 8.06 (1H, m), 7.67-7.77 (4H, m), 7.62 (1H, br s), 7.20-7.43 (11H, m), 7.13 (2H, d, J=8 Hz), 7.07 (2H, d, J=8 Hz), 6.73 (1H, d, J=7 Hz), 6.13 (1H, br s), 5.59 (1H, m), 5.29 (2H, s), 5.27 (1H, br s), 5.07 (2H, s), 4.67 (1H, m), 3.15 (2H, d, J=7 Hz), 2.57 (1H, dd, J=14, 4 Hz), 2.48 (1H, dd, J=14, 7 Hz), 2.32 (3H, s), 2.07-2.28 (2H, m), 1.43-1.88 (4H, m)

ESI-MS: 710 [M+H]

### EXAMPLE 52

(3S)-3-[N-(2-Pyridylmethyl)-{(2S)-5-benzyloxycarbonyl-2-(2-quinolylcarbonylamino)pentanoyl}amino]hexadecanamide

ESI-MS: 750 [M+H]

### EXAMPLE 53

(3S)-3-[N-Benzyl-{(2S)-5-benzyloxycarbonyl-2-(2-quinolylcarbonylamino)pentanoyl}amino]hexadecanamide

ESI-MS: 749 [M+H]

### EXAMPLE 54

(3S)-3-[N-(n-Pentyl)-{(2S)-5-benzyloxycarbonyl-2-(2-quinolylcarbonylamino)pentanoyl}amino]hexadecanamide

ESI-MS: 729 [M+H]

### EXAMPLE 55

(3S)-3-[N-Phenethyl-{(2S)-5-benzyloxycarbonyl-2-(2-quinolylcarbonylamino)pentanoyl}amino]hexadecanamide

ESI-MS: 763 [M+H]

### EXAMPLE 56

(3S)-3-[N-(n-Butyl)-{(2S)-5-benzyloxycarbonyl-2-(2-quinolylcarbonylamino)pentanoyl}amino]hexadecanamide ESI-MS: 737 [M+H]

### EXAMPLE 57

(3S)-3-[N-Ethyl-{(2S)-5-benzyloxycarbonyl-2-(2-quinolylcarbonylamino)pentanoyl}amino]hexadecanamide

ESI-MS: 687 [M+H]

### EXAMPLE 58

(3S)-3-[N-(n-Propyl)-{(2S)-2-(1-benzylindol-3-ylcarbonylamino)-5-benzyloxycarbonylpentanoyl}amino]-4-(2-naphthyl)butanamide

ESI-MS: 737 [M+H]

### EXAMPLE 59

(3S)-3-[N-(n-Propyl)-{(2S)-5-benzyloxycarbonyl-2-(1-(1-naphthylmethyl)indol-3-ylcarbonylamino)pentanoyl}amino]-4-(2-naphthyl)butanamide

ESI-MS 787 [M+H]

### EXAMPLE 60

(3S)-3-[N-(n-Pentyl)-{(2S)-5-benzyloxycarbonyl-2-(1-benzylindol-3-ylcarbonylamino)pentanoyl}amino]-4-(2-naphthyl)butanamide

ESI-MS: 765 [M+H]

### EXAMPLE 61

(3S)-3-[N-(n-Pentyl)-{(2S)-5-benzyloxycarbonyl-2-(1-(1-naphthylmethyl)indol-3-ylcarbonylamino)pentanoyl}amino]-4-(2-naphthyl)butanamide

ESI-MS 815 [M+H]

### EXAMPLE 62

(3S)-3-[N-Isobutyl-{(2S)-5-benzyloxycarbonyl-2-(2-quinolylcarbonylamino)pentanoyl}amino]hexadecanamide

ESI-MS: 715 [M+H]

### EXAMPLE 63

(3S)-3-[N-Isopentyl-{(2S)-5-benzyloxycarbonyl-2-(2-quinolylcarbonylamino)pentanoyl}amino]hexadecanamide

ESI-MS: 729 [M+H]

### EXAMPLE 64

(3S)-3-[N-Ethyl-{(2S)-5-benzyloxycarbonyl-2-(1-(1-benzylindol-3-ylcarbonylamino)pentanoyl}amino]-4-(2-naphthyl)butanamide

ESI-MS: 723 [M+H]

### EXAMPLE 65

(3S)-3-[N-Ethyl-{(2S)-5-benzyloxycarbonyl-2-(1-naphthylmethyl)indol-3-ylcarbonylamino)pentanoyl}amino]-4-(2-naphthyl)butanamide

ESI-MS: 773 [M+H]

### EXAMPLE 66

(3S)-3-[N-Ethyl-{(2S)-5-benzyloxycarbonyl-2-(1-(2-chlorobenzyl)indol-3-ylcarbonylamino)pentanoyl}amino]-4-(2-naphthyl)butanamide

ESI-MS: 757 [M+H]

### EXAMPLE 67

(3S)-3-[N-(n-Butyl)-{(2S)-5-benzyloxycarbonyl-2-(1-benzylindol-3-ylcarbonylamino)pentanoyl}amino]-4-(2-naphthyl)butanamide

ESI-MS: 751 [M+H]

### EXAMPLE 68

(3S)-3-[N-(n-Butyl)-{(2S)-5-benzyloxycarbonyl-2-(1-(2-chlorobenzyl)indol-3-ylcarbonylamino)pentanoyl}amino]-4-(2-naphthyl)butanamide

ESI-MS 785 [M+H]

### EXAMPLE 69

(3S)-3-[N-(n-Propyl)-{(2S)-2-(1-benzylindol-3-ylcarbonylamino)-5-benzyloxycarbonylpentanoyl}amino]-4-(4-methylphenyl)butanamide

### EXAMPLE 70

(3S)-3-[N-(n-Propyl)-{(2S)-5-benzyloxycarbonyl-2-(1-(2-chlorobenzyl)indol-3-ylcarbonylamino)pentanoyl}amino]-4-(4-methylphenyl)butanamide

### EXAMPLE 71

(3S)-3-[N-(n-Propyl)-{(2S)-5-benzyloxycarbonyl-2-(1-benzylindol-3-ylcarbonylamino)pentanoyl}amino]-4-(4-n-butylphenyl)butanamide ESI-MS: 743 [M+H]

### EXAMPLE 72

(3S)-3-[N-(n-Propyl)-{(2S)-5-benzyloxycarbonyl-2-(1-(2-chlorobenzyl)indol-3-ylcarbonylamino)pentanoyl}amino]-4-(4-n-butylphenyl)butanamide

ESI-MS 777 [M+H]

### EXAMPLE 73

(3S)-3-[N-(n-Butyl)-{(2S)-5-benzyloxycarbonyl-2-(1-benzylindol-3-ylcarbonylamino)pentanoyl}amino]-4-(4-n-butylphenyl)butanamide

ESI-MS: 757 [M+H]

### EXAMPLE 74

(3S)-3-[N-(n-Butyl)-{(2S)-5-benzyloxycarbonyl-2-(1-(2-chlorobenzyl)indol-3-ylcarbonylamino)pentanoyl}amino]-4-(4-n-butylphenyl)butanamide

ESI-MS 791 [M+H]

### EXAMPLE 75

(3S)-3-[N-(n-Propyl)-{(2S)-4-benzyloxycarbonyl-2-(1-(2-chlorobenzyl)indol-3-ylcarbonylamino)butanoyl}amino]-4-(2-naphthyl)butanamide

ESI-MS 757 [M+H]

### EXAMPLE 76

(3S)-3-[N-(n-Butyl)-{(2S)-5-methoxycarbonyl-2-(2-quinolylcarbonylamino)pentanoyl}amino]-4-(4-n-heptylphenyl)butanamide

ESI-MS: 667 [M+H]

### EXAMPLE 77

(3S)-3-[N-(n-Butyl)-{(2S)-5-methoxycarbonyl-2-(1-(2-chlorobenzyl)indol-3-ylcarbonylamino)pentanoyl}amino]-4-(4-n-heptylphenyl)butanamide

ESI-MS: 757 [M+H]

### EXAMPLE 78

(3S)-3-[(2S)-5-Benzyloxycarbonyl-2-(3-quinolylcarbonylamino)pentanoyl]oxy-10-phenyldecanamide

NMR (CDCl₃, δ): 9.34 (1H, s), 8.62 (1H, s), 8.16 (1H, d, J=10 Hz), 7.90 (1H, d, J=10 Hz), 7.82 (1H, m), 7.64 (1H, m), 7.1-7.4 (11H, m), 5.94 (1H, br s), 5.50 (1H, br s), 5.35 (1H, m), 5.12 (2H, s), 4.80 (1H, m), 2.4-2.7 (6H, m), 1.5-2.15 (8H, m), 1.2-1.4 (8H, m)

NMS 652 [M+H]

### EXAMPLE 79

(3S)-3-[(2S)-5-Benzyloxycarbonyl-2-(3-quinolylcarbonylamino)pentanoyl]oxy-4-(2-naphthyl)butanamide

NMR (CDCl₃, δ): 9.32 (1H, d, J=2 Hz), 8.56 (1H, d, J=2 Hz), 8.15 (1H, d, J=15 Hz), 7.6-7.9 (7H, m), 7.25-7.4 (8H, m), 5.9 (1H, br s), 5.62 (1H, m), 5.40 (1H, br s), 5.10 (2H, s), 4.68 (1H, m), 3.15 (2H, d, J=7 Hz), 2.45-2.6 (2H, m), 2.15-2.4 (2H, m), 1.75-1.9 (2H, m), 1.5-1.7 (2H, m)

FAB-MS 618 [M+H]

### EXAMPLE 80

(3S)-3-[(2S)-5-Benzyloxycarbonyl-2-(isoquinolin-3-ylcarbonylamino)pentanoyl]oxy-4-(2-naphthyl)butanamide

NMR (CDCl₃, δ): 9.2 (1H, s), 8.66 (1H, d, J=15 Hz), 8.56 (1H, s), 8.1 (1H, d, J=15 Hz), 8.0 (1H, d, J=15 Hz), 7.6-7.85 (5H, m), 7.25-7.4 (8H, m), 6.0 (1H, br s), 5.62 (1H, m), 5.35 (1H, br s), 5.10 (2H, s), 4.75 (1H, m), 3.15 (2H, d, J=7 Hz), 2.45-2.6 (2H, m), 2.15-2.4 (2H, m), 1.75-1.9 (2H, m), 1.5-1.7 (2H, m)

FAB-MS: 618 [M+H]

### EXAMPLE 81

(3S)-3-[(2S)-5-Benzyloxycarbonyl-2-(isoquinolin-1-ylcarbonylamino)pentanoyl]oxy-4-(2-naphthyl)butanamide

NMR (CDCl₃, δ): 9.5 (1H, d, J=15 Hz), 8.65 (1H, d, J=15 Hz), 8.5 (1H, d, J=10 Hz), 7.6-7.9 (10H, m), 7.2-7.4 (5H, m), 6.04 (1H, br s), 5.58 (1H, m), 5.42 (1H, br s), 5.05 (2H, s), 4.7 (1H, m), 3.12 (2H, m), 2.5 (2H, m), 2.2-2.3 (2H, m), 1.5-1.9 (2H, m)

FAB-MS: 618 [M+H]

### EXAMPLE 82

(3S)-3-[(2S)-2-(2-Benzylbenzoyl)amino-5-benzyloxycarbonylpentanoyl]oxy-4-(2-naphthyl)butanamide

NMR (CDCl₃, δ): 7.05-7.8 (21H, m), 6.22 (1H, d, J=10 Hz), 5.82 (1H, br s), 5.55 (1H, m), 5.26 (1H, m), 5.05 (2H, s), 4.5 (1H, m), 4.2 (2H, ABq), 3.15 (2H, t, J=7 Hz), 2.4-2.55 (2H, m), 2.05-2.25 (2H, m), 1.3-1.7 (4H, m)

ESI-MS 657 [M+H]

### EXAMPLE 83

(3S)-3-[(2S)-5-Benzyloxycarbonyl-2-(2-naphthylcarbonylamino)pentanoyl]oxy-4-(2-naphthyl)butanamide

NMR (DMSO-d₆, δ): 8.9 (1H, d, J=10 Hz), 8.52 (1H, s), 7.3-8.05 (14H, m), 6.85 (1H, br s), 5.46 (1H, m), 5.08 (2H, s), 4.44 (1H, m), 3.0-3.2 (2H, m), 2.3-2.4 (4H, m), 1.5-1.9 (4H, m)

ESI-MS: 617 [M+H]

### EXAMPLE 84

(3S)-3-[(2S)-2-Benzoylamino-5-benzyloxycarbonylpentanoyl]oxy-4-(2-naphthyl)butanamide

NMR (CDCl₃, δ): 8.16 (1H, d, J=10 Hz), 7.3-7.85 (11H, m), 6.9 (1H, d, J=8 Hz), 5.94 (1H, br s), 5.6 (1H, m), 5.35 (1H, br s), 5.1 (2H, s), 4.12 (1H, m), 3.12 (2H, d, J=7 Hz), 2.1-2.6 (4H, m), 1.45-1.9 (4H, m)

ESI-MS: 567 [M+H]

### EXAMPLE 85

(3S)-3-[(2S)-5-Benzyloxycarbonyl-2-(2-phenethylbenzoyl)aminopentanoyl]oxy-4-(2-naphthyl)butanamide

NMR (CDCl₃, δ): 7.1-7.8 (21H, m), 6.25 (1H, d, J=10 Hz), 5.78 (1H, br s), 5.6 (1H, m), 5.26 (1H, br s), 5.08 (2H, s), 4.6 (1H, m), 3.14 (2H, d, J=7 Hz), 3.0-3.1 (2H, m), 2.85-2.95 (2H, m), 2.44 (2H, d, J=7 Hz), 2.1-2.3 (2H, m), 1.45-1.8 (4H, m)

ESI-MS: 671 [M+H]

### EXAMPLE 86

(3S)-3-[(2S)-2-(3-Benzylbenzoyl)amino-5-benzyloxycarbonylpentanoyl]oxy-4-(2-naphthyl)butanamide

NMR (CDCl₃, δ): 8.1 (1H, m), 7.1-7.75 (20H, m), 6.92 (1H, d, J=8 Hz), 5.92 (1H, br s), 5.55 (1H, m), 5.36 (1H, br s), 5.05 (2H, s), 4.56 (1H, m), 4.02 (2H, s), 3.1 (2H, d, J=7 Hz), 2.4-2.55 (2H, m), 2.05-2.3 (2H, m), 1.4-1.8 (4H, m)

ESI-MS: 657 [M+H]

### EXAMPLE 87

(3S)-3-[(2S)-2-(3-Benzylnaphthalen-2-ylcarbonyl)amino-5-benzyloxycarbonylpentanoyl]oxy-4-(6-ethyl-2-naphthyl)butanamide

NMR (CDCl₃, δ): 7.05-7.85 (22H, m), 6.32 (1H, d, J=10 Hz), 5.82 (1H, br s), 5.55 (1H, m), 5.26 (1H, m), 5.05 (2H, s), 4.45 (1H, m), 4.32 (2H, ABq), 3.15 (2H, t, J=7 Hz), 2.75 (2H, a, J=7 Hz), 2.4-2.55 (2H, m), 2.05-2.25 (2H, m), 1.3-1.7 (4H, m), 1.26 (3H, t, J=7 Hz)

ESI-MS: 735 [M+H]

### EXAMPLE 88

3-[N-Methyl-{(2S)-2-(3-benzylnaphthalen-2-ylcarbonyl)amino-5-benzyloxycarbonylpentanoyl}amino]propanamide

ESI-MS: 580 [M+H]

### EXAMPLE 89

(3S)-3-[N-(n-Pentyl)-{(2S)-2-(1-benzylindol-3-ylcarbonyl)amino-5-benzyloxycarbonylpentanoyl}amino]-dodecanamide

ESI-MS 751 [M+H]

### EXAMPLE 90

(3S)-3-[N-(n-Propyl)-{(2S)-2-(1-benzylindol-3-yl-carbonyl)amino-5-methoxycarbonylpentanoyl}amino]dodecanamide

ESI-MS: 647 [M+H]

### EXAMPLE 91

(3S)-3-[N-(n-Propyl)-{(2S)-2-(1-(2-chlorobenzyl)indol-3-ylcarbonyl)amino-5-methoxycarbonylpentanoyl}amino]-dodecanamide

ESI-MS: 681 [M+H]

### EXAMPLE 92

(3S)-3-[N-(n-Pentyl)-{(2S)-2-(1-benzylindol-3-yl-carbonyl)amino-5-benzyloxycarbonylpentanoyl}amino]nonanamide ESI-MS: 709 [M+H]

### EXAMPLE 93

(3S)-3-[N-(n-Butyl)-{(2S)-2-(1-benzylindol-3-yl-carbonyl)amino-5-benzyloxycarbonylpentanoyl}amino]nonanamide

ESI-MS: 695 [M+H]

### EXAMPLE 94

(3S)-3-[N-(n-Butyl)-{(2S)-5-benzyloxycarbonyl-2-(1-(2-chlorobenzyl)indol-3-ylcarbonylamino)pentanoyl}amino]-nonanamide

ESI-MS: 729 [M+H]

### EXAMPLE 95

(3S)-3-[N-(n-Propyl)-{(2S)-2-(1-benzylindol-3-yl-carbonyl)amino-5-benzyloxycarbonylpentanoyl}amino]nonanamide

ESI-MS 681 [M+H]

### EXAMPLE 96

(3S)-3-[N-(n-Propyl)-{(2S)-5-benzyloxycarbonyl-2-[(1-(1-naphthylmethyl)indol-3-ylcarbonyl)amino]pentanoyl}amino]-nonanamide

ESI-MS: 731 [M+H]

### EXAMPLE 97

(3S)-3-[N-(n-Propyl)-{(2S)-5-benzyloxycarbonyl-2-[(1-(2-pyridylmethyl)indol-3-ylcarbonyl)amino]pentanoyl}amino]-nonanamide

ESI-MS: 682 [M+H]

### EXAMPLE 98

(3S)-3-[N-(n-Propyl)-{(2S)-5-benzyloxycarbonyl-2-[(1-(2-chlorobenzyl)indol-3-ylcarbonyl)amino]pentanoyl}amino]-nonanamide

ESI-MS 715 [M+H]

### EXAMPLE 99

(3S)-3-[N-(n-Propyl)-{(2S)-5-benzyloxycarbonyl-2-[(1-(3-chlorobenzyl)indol-3-ylcarbonyl)amino]pentanoyl}amino]-nonanamide

ESI-MS 715 [M+H]

### EXAMPLE 100

(3S)-3-[N-Ethyl-{(2S)-2-(1-(2-chlorobenzyl)indol-3-ylcarbonyl)amino-5-methoxycarbonylpentanoyl}amino]nonanamide

ESI-MS 625 [M+H]

### EXAMPLE 101

(3S)-3-[N-Ethyl-{(2S)-2-(1-benzylindol-3-ylcarbonyl)amino-5-methoxycarbonylpentanoyl}amino]nonanamide

ESI-MS: 591 [M+H]

### EXAMPLE 102

(3S)-3-[N-(n-Propyl)-{(2S)-2-(1-(2-chlorobenzyl)indol-3-ylcarbonyl)amino-5-benzyloxycarbonylpentanoyl}amino]-heptanamide

ESI-MS 687 [M+H]

### EXAMPLE 103

(3S)-3-[N-(n-Propyl)-{(2S)-2-(1-(1-naphthylmethyl)indol-3-ylcarbonyl)amino-5-benzyloxycarbonylpentanoyl}amino]-heptanamide

ESI-MS: 703 [M+H]

### EXAMPLE 104

(3S)-3-[N-(n-Butyl)-{(2S)-2-(1-(2-chlorobenzyl)indol-3-ylcarbonyl)amino-5-methoxycarbonylpentanoyl}amino]-heptanamide

ESI-MS: 625 [M+H]

### EXAMPLE 105

(3S)-3-[N-(n-Butyl)-{(2S)-2-(1-(1-naphthylmethyl)indol-3-ylcarbonyl)amino-5-methoxycarbonylpentanoyl}amino]-heptanamide

ESI-MS: 641 [M+H]

### EXAMPLE 106

(3S)-3-[N-(n-Propyl)-{(2S)-5-benzyloxycarbonyl-2-(1-(2-pyridylmethyl)indol-3-ylcarbonylamino)pentanoyl}amino]-4-(6-ethyl-2-naphthyl)butanamide

ESI-MS 766 [M+H]

### EXAMPLE 107

(3S)-3-[N-(n-Propyl)-{(2S)-2-(1-benzylindol-3-ylcarbonyl)amino-5-benzyloxycarbonylpentanoyl}amino]-4-(6-ethyl-2-naphthyl)butanamide

ESI-MS: 765 [M+H]

### EXAMPLE 108

(3S)-3-[N-(n-Butyl)-{(2S)-5-benzyloxycarbonyl-2-[(1-benzylindol-3-ylcarbonyl)amino]pentanoyl}amino]-4-(6-ethyl-2-naphthyl)butanamide

ESI-MS: 779 [M+H]

### EXAMPLE 109

(3S)-3-[N-(n-Butyl)-{(2S)-5-benzyloxycarbonyl-2-[(1-(2-chlorobenzyl)indol-3-ylcarbonyl)amino]pentanoyl}amino]-4-(6-ethyl-2-naphthyl)butanamide

ESI-MS: 813 [M+H]

### EXAMPLE 110

(3S)-3-[N-(2-Pyridylmethyl)-{(2S)-5-benzyloxycarbonyl-2-(tert-butoxycarbonylamino)pentanoyl}amino]hexadecanamide

ESI-MS: 695 [M+H]

### EXAMPLE 111

(3S)-3-[N-Benzyl-{(2S)-5-benzyloxycarbonyl-2-(tert-butoxycarbonylamino)pentanoyl}amino]hexadecanamide

ESI-MS 694 [M+H]

### EXAMPLE 112

(3S)-3-[N-(n-Pentyl)-{(2S)-5-benzyloxycarbonyl-2-(tert-butoxycarbonylamino)pentanoyl}amino]hexadecanamide

ESI-MS 674 [M+H]

### EXAMPLE 113

(3S)-3-[N-Phenethyl-{(2S)-5-benzyloxycarbonyl-2-(tert-butoxycarbonylamino)pentanoyl}amino]hexadecanamide

ESI-MS: 708 [M+H]

### EXAMPLE 114

(3S)-3-[N-(n-Butyl)-{(2S)-5-benzyloxycarbonyl-2-(tert-butoxycarbonylamino)pentanoyl}amino]hexadecanamide

ESI-MS: 660 [M+H]

### EXAMPLE 115

(3S)-3-[N-Ethyl-{(2S)-5-benzyloxycarbonyl-2-(tert-butoxycarbonylamino)pentanoyl}amino]hexadecanamide

ESI-MS: 632 [M+H]

### EXAMPLE 116

(3S)-3-[N-(n-Propyl)-{(2S)-5-benzyloxycarbonyl-2-(tert-butoxycarbonylamino)pentanoyl}amino]-4-(2-naphthyl)butanamide

ESI-MS 604 [M+H]

### EXAMPLE 117

(3S)-3-[N-(n-Pentyl)-{(2S)-5-benzyloxycarbonyl-2-(tert-butoxycarbonylamino)pentanoyl}amino]-4-(2-naphthyl)butanamide

ESI-MS: 632 [M+H]

### EXAMPLE 118

(3S)-3-[(N-Isobutyl-{(2S)-5-benzyloxycarbonyl-2-(tert-butoxycarbonylamino)pentanoyl}amino]hexadecanamide

ESI-MS: 660 [M+H]

### EXAMPLE 119

(3S)-3-[N-Isopentyl-{(2S)-5-benzyloxycarbonyl-2-(tert-butoxycarbonylamino)pentanoyl}amino]hexadecanamide

ESI-MS: 674 [M+H]

### EXAMPLE 120

(3S)-3-[N-Ethyl-{(2S)-5-benzyloxycarbonyl-2-(tert-butoxycarbonylamino)pentanoyl}amino]-4-(2-naphthyl)butanamide

ESI-MS: 590 [M+H]

### EXAMPLE 121

(3S)-3-[N-(n-Butyl)-{(2S)-5-benzyloxycarbonyl-2-(tert-butoxycarbonylamino)pentanoyl}amino]-4-(2-naphthyl)butanamide

ESI-MS 618 [M+H]

### EXAMPLE 122

(3S)-3-[N-(n-Propyl)-{(2S)-5-benzyloxycarbonyl-2-(tert-butoxycarbonylamino)pentanoyl}amino]-4-(4-methylphenyl)butanamide

ESI-MS 568 [M+H]

### EXAMPLE 123

(3S)-3-[(N-(n-Propyl)-{(2S)-5-benzyloxycarbonyl-2-(tert-butoxycarbonylamino)pentanoyl}amino]-4-(4-n-butylphenyl)butanamide

ESI-MS 610 [M+H]

### EXAMPLE 124

(3S)-3-[N-(n-Propyl)-{(2S)-4-benzyloxycarbonyl-2-(tert-butoxycarbonylamino)butanoyl}amino]-4-(2-naphthyl)butanamide

ESI-MS: 590 [M+H]

### EXAMPLE 125

(3S)-3-[N-(n-Butyl)-{(2S)-2-(tert-butoxycarbonyl)amino-5-methoxycarbonylpentanoyl}amino]-4-(4-n-heptylphenyl)butanamide

ESI-MS 590 [M+H]

### EXAMPLE 126

3-[N-Methyl-{(2S)-5-benzyloxycarbonyl-2-(tert-butoxycarbonylamino)pentanoyl}amino]propanamide

ESI-MS 436 [M+H]

### EXAMPLE 127

(3S)-3-[N-(n-Pentyl)-{(2S)-5-benzyloxycarbonyl-2-(tert-butoxycarbonylamino)pentanoyl}amino]dodecanamide

ESI-MS 618 [M+H]

### EXAMPLE 128

(3S)-3-[N-(n-Propyl)-{(2S)-2-(tert-butoxycarbonyl)amino-5-methoxycarbonylpentanoyl}amino]dodecanamide

ESI-MS: 514 [M+H]

### EXAMPLE 129

(3S)-3-[N-(n-Pentyl)-{(2S)-5-benzyloxycarbonyl-2-(tert-butoxycarbonylamino)pentanoyl}amino]nonanamide

ESI-MS 576 [M+H]

### EXAMPLE 130

(3S)-3-[N-(n-Butyl)-{(2S)-5-benzyloxycarbonyl-2-(tert-butoxycarbonylamino)pentanoyl}amino]nonanamide

ESI-MS 562 [M+H]

### EXAMPLE 131

(3S)-3-[N-(n-Propyl)-{(2S)-5-benzyloxycarbonyl-2-(tert-butoxycarbonylamino)pentanoyl}amino]nonanamide

ESI-MS: 548 [M+H]

### EXAMPLE 132

(3S)-3-[(N-Ethyl-{(2S)-2-(tert-butoxycarbonyl)amino-5-methoxycarbonylpentanoyl}amino]nonanamide

ESI-MS: 458 [M+H]

### EXAMPLE 133

(3S)-3-[N-(n-Propyl)-{(2S)-5-benzyloxycarbonyl-2-(tert-butoxycarbonylamino)pentanoyl}amino]heptanamide

ESI-MS: 520 [M+H]

### EXAMPLE 134

(3S)-3-[N-(n-Butyl)-{(2S)-2-(tert-butoxycarbonyl)amino-5-methoxycarbonylpentanoyl}amino]heptanamide

ESI-MS: 458 [M+H]

### EXAMPLE 135

(3S)-3-[N-(n-Propyl)-{(2S)-5-benzyloxycarbonyl-2-(tert-butoxycarbonylamino)pentanoyl}amino]-4-(6-ethyl-2-naphthyl)butanamide

ESI-MS 632 [M+H]

### EXAMPLE 136

(3S)-3-[N-(n-Butyl)-{(2S)-5-benzyloxycarbonyl-2-(tert-butoxycarbonylamino)pentanoyl}amino]-4-(6-ethyl-2-naphthyl)butanamide

ESI-MS: 646 [M+H]

The following compounds (Examples 137 to 194) were obtained according to a similar manner to that of Example 21.

### EXAMPLE 137

(3S)-3-[N-(n-Propyl)-{(2S)-4-carboxy-2-[(1-(2-chlorobenzyl)indol-3-ylcarbonyl)amino]butanoyl}amino]-4-(2-naphthyl)butanamide

ESI-MS: 667 [M+H]

mp: 85-92° C.

### EXAMPLE 138

(3S)-3-[N-(n-Butyl)-{(2S)-5-carboxy-2-[(1-(2-chlorobenzyl)indol-3-ylcarbonyl)amino]pentanoyl}amino]-4-(4-n-butylphenyl)butanamide

ESI-MS: 701 [M+H]

mp 166-168° C.

### EXAMPLE 139

(3S)-3-[N-(n-Butyl)-{(2S)-5-carboxy-2-[(1-benzylindol-3-ylcarbonyl)amino]pentanoyl}amino]-4-(4-n-butylphenyl)butanamide

ESI-MS 667 [M+H]

mp 77-82° C.

### EXAMPLE 140

(3S)-3-[N-(n-Propyl)-{(2S)-5-carboxy-2-[(1-(2-chlorobenzyl)indol-3-ylcarbonyl)amino]pentanoyl}amino]-4-(4-n-butylphenyl)butanamide

ESI-MS: 685 [M−H]

mp: 83-87° C.

### EXAMPLE 141

(3S)-3-[N-(n-Propyl)-{(2S)-5-carboxy-2-[(1-benzylindol-3-ylcarbonyl)amino]pentanoyl}amino]-4-(4-n-butylphenyl)butanamide

ESI-MS: 651 [M−H]

mp: 82-88° C.

### EXAMPLE 142

(3S)-3-[N-(n-Propyl)-{(2S)-5-carboxy-2-[(1-(2-chlorobenzyl)indol-3-ylcarbonyl)amino]pentanoyl}amino]-4-(4-methylphenyl)butanamide

ESI-MS: 643 [M−H]

mp: 79-96° C.

### EXAMPLE 143

(3S)-3-[N-(n-Propyl)-{(2S)-2-(1-benzylindol-3-ylcarbonyl)amino-5-carboxypentanoyl}amino]-4-(4-methylphenyl)butanamide

ESI-MS: 609 [M−H]

mp: 83-92° C.

### EXAMPLE 144

(3S)-3-[N-(n-Butyl)-{(2S)-5-carboxy-2-[(1-(2-chlorobenzyl)indol-3-ylcarbonyl)amino]pentanoyl}amino]-4-(2-naphthyl)butanamide

ESI-MS: 693 [M−H]

mp: 104-110° C.

### EXAMPLE 145

(3S)-3-[N-(n-Butyl)-{(2S)-5-carboxy-2-[(1-benzylindol-3-ylcarbonyl)amino]pentanoyl}amino]-4-(2-naphthyl)butanamide

ESI-MS: 659 [M−H]

Mp: 90-96° C.

### EXAMPLE 146

(3S)-3-[N-Ethyl-{(2S)-5-carboxy-2-[(1-(2-chlorobenzyl)indol-3-ylcarbonyl)amino]pentanoyl{amino]-4-(2-naphthyl)butanamide

ESI-MS: 665 [M−H]

mp 110-114° C.

### EXAMPLE 147

(3S)-3-[N-Ethyl-{(2S)-5-carboxy-2-[(1-(1-naphthylmethyl)indol-3-ylcarbonyl)amino]pentanoyl}amino]-4-(2-naphthyl)butanamide

ESI-MS: 681 [M−H]

mp: 118-126° C.

### EXAMPLE 148

(3S)-3-[N-Isopentyl-((2S)-5-carboxy-2-(2-quinolylcarbonylamino)pentanoyl}amino]hexadecanamide

ESI-MS 639 [M+H]

### EXAMPLE 149

(3S)-3-[N-Isobutyl-{(2S)-5-carboxy-2-(2-quinolylcarbonylamino)pentanoyl}amino]hexadecanamide

ESI-MS: 625 [M+H]

mp: 65-66° C.

### EXAMPLE 150

(3S)-3-[N-Phenethyl-{(2S)-5-carboxy-2-(2-quinolylcarbonylamino)pentanoyl}amino]hexadecanamide

ESI-MS 673 [M+H]

### EXAMPLE 151

(3S)-3-[N-Ethyl-{(2S)-5-carboxy-2-(1-benzylindol-3-ylcarbonylamino)pentanoyl}amino]-4-(2-naphthyl)butanamide

ESI-MS: 631 [M−H]

mp: 110-112° C.

### EXAMPLE 152

(3S)-3-[N-(n-Pentyl)-{(2S)-5-carboxy-2-[(1-benzylindol-3-ylcarbonyl)amino]pentanoyl}amino]-4-(2-naphthyl)butanamide

ESI-MS: 675 [M+H]

mp: 89-93° C.

### EXAMPLE 153

(3S)-3-[N-(n-Pentyl)-{(2S)-5-carboxy-2-[(1-(1-naphthylmethyl)indol-3-ylcarbonyl)amino]pentanoyl}amino]-4-(2-naphthyl)butanamide

ESI-MS: 725 [M+H]

mp 112-116° C.

### EXAMPLE 154

(3S)-3-[N-(n-Propyl)-{(2S)-5-carboxy-2-[(1-(1-naphthylmethyl)indol-3-ylcarbonyl)amino]pentanoyl}amino]-4-(2-naphthyl)butanamide

ESI-MS: 697 [M+H]

mp: 123-126° C.

### EXAMPLE 155

(3S)-3-[N-(n-Propyl)-{(2S)-2-(1-benzylindol-3-ylcarbonyl) amino-5-carboxypentanoyl}amino]-4-(2-naphthyl)butanamide

ESI-MS: 647 [M+H]

mp: 91-94° C.

### EXAMPLE 156

(3S)-3-[N-(2-Pyridylmethyl)-{(2S)-5-carboxy-2-(2-quinolylcarbonylamino)pentanoyl}amino]hexadecanamide dihydrochloride

ESI-MS: 660 [M+H]

mp: 74-81° C.

### EXAMPLE 157

(3S)-3-[N-Ethyl-{(2S)-5-carboxy-2-(2-quinolylcarbonylamino)pentanoyl}amino]hexadecanamide

ESI-MS 660 [M+H]

### EXAMPLE 158

(3S)-3-[N-(n-Propyl)-{(2S)-5-carboxy-2-(2-quinolylcarbonylamino)pentanoyl}amino]hexadecanamide hydrochloride

mp: 59-62° C.

### EXAMPLE 159

(3S)-3-[N-(n-Butyl)-{(2S)-5-carboxy-2-(2-quinolylcarbonylamino)pentanoyl}amino]hexadecanamide

ESI-MS 625 [M−H]

### EXAMPLE 160

(3S)-3-[N-(n-Pentyl)-{(2S)-5-carboxy-2-(2-quinolylcarbonylamino)pentanoyl}amino]hexadecanamide

ESI-MS 639 [M+H]

### EXAMPLE 161

(3S)-3-[N-Benzyl-((2S)-5-carboxy-2-(2-quinolylcarbonylamino)pentanoyl}amino]hexadecanamide

ESI-MS: 659 [M+H]

### EXAMPLE 162

(3S)-3-[N-(2-Pyridylmethyl)-{(2S)-5-carboxy-2-(2-quinolylcarbonylamino)pentanoyl}amino]hexadecanamide

ESI-MS 660 [M+H]

### EXAMPLE 163

(3S)-3-[(2S)-5-Carboxy-2-[{1-(4-methylbenzyl)indol-3-ylcarbonyl}amino]pentanoyl]oxy-4-(2-naphthyl)butanamide

NMR (DMSO-d₆, δ): 8.13-8.28 (3H, m), 7.70-7.86 (4H, m), 7.34-7.57 (5H, m), 7.08-7.23 (6H, m), 6.85 (1H, br s), 5.42 (2H, s), 5.39 (1H, m), 4.40 (1H, m), 2.96-3.16 (2H, m), 2.29-2.38 (2H, m), 2.25 (3H, s), 2.11-2.20 (2H, m), 1.44-1.83 (4H, m)

ESI-MS 620 [M+H]

### EXAMPLE 164

(3S)-3-[(2S)-5-Carboxy-2-[{1−(4-chlorobenzyl)indol-3-ylcarbonyl}amino]pentanoyl]oxy-4-(2-naphthyl)butanamide

NMR (DMSO-d₆, δ): 8.12-8.28 (3H, m), 7.72-7.87 (4H, m), 7.32-7.57 (6H, m), 7.10-7.31 (4H, m), 6.85 (1H, br s), 5.49 (2H, s), 5.42 (1H, m), 4.42 (1H, m), 2.95-3.16 (2H, m), 2.29-2.38 (2H, m), 2.12-2.22 (2H, m), 1.45-1.84 (4H, m)

ESI-MS: 640 [M+H]

### EXAMPLE 165

(3S)-3-[(2S)-5-Carboxy-2-[{1-(4-methylbenzyl)indol-2-ylcarbonyl}amino]pentanoyl]oxy-4-(2-naphthyl)butanamide

ESI-MS: 620 [M+H]

mp: 86-90° C.

### EXAMPLE 166

(3S)-3-[(2S)-5-Carboxy-2-(2-quinolylcarbonylamino)pentanoyl]oxy-5-(2-naphthyl)pentanamide

NMR (DMSO-d₆, δ): 9.12 (1H, d, J=8 Hz), 8.60 (1H, d, J=8 Hz), 8.22 (1H, t, J=8 Hz), 8.20 (1H, d, J=9 Hz), 8.12 (1H, d, J=8 Hz), 7.90 (1H, t, J=8 Hz), 7.68-7.86 (4H, m), 7.62 (1H, br s), 7.38-7.46 (3H, m), 6.88 (1H, br s), 5.20 (1H, m), 4.54 (1H, m), 2.70-2.86 (2H, m), 2.42-2.49 (2H, m), 2.27 (3H, t, J=7 Hz), 1.87-2.04 (4H, m), 1.53-1.67 (2H, m)

### EXAMPLE 167

(3S)-3-[(2S)-2-(3-Benzylnaphthalen-2-ylcarbonyl)amino-5-carboxypentanoyl]oxy-4-(4-n-heptylphenyl)butanamide

NMR (DMSO-d₆, δ): 8.82 (1H, d, J=7 Hz), 7.95 (1H, m), 7.93 (1H, s), 7.85 (1H, m), 7.75 (1H, s), 7.47-7.59 (2H, m), 7.38 (1H, br s), 7.05-7.28 (9H, m), 6.86 (1H, br s), 5.32 (1H, m), 4.37 (1H, d, J=14 Hz), 4.33 (1H, m), 4.29 (1H, d, J=14 Hz), 2.78-2.96 (2H, m), 2.24-2.40 (2H, m), 2.12-2.21 (2H, m), 1.42-1.75 (6H, m), 1.13-1.35 (8H, m), 0.78-0.88 (3H, m)

ESI-MS 665 [M+H]

### EXAMPLE 168

(3S)-3-[(2S)-5-Carboxy-2-[2-((2E)-2-methoxycarbonylvinyl)benzoylamino]pentanoyl]oxy-4-(2-naphthyl)butanamide

NMR (DMSO-d₆, δ): 8.92 (1H, d, J=7 Hz), 7.77-7.98 (6H, m), 7.42-7.57 (6H, m), 7.37 (1H, br s), 6.88 (1H, br s), 6.59 (1H, d, J=16 Hz), 5.43 (1H, m), 4.23 (1H, m), 3.66 (3H, s), 3.16 (1H, dd, J=14, 6 Hz), 3.06. (1H, dd, J=14, 6 Hz), 2.37 (2H, d, J=7 Hz), 2.15 (3H, t, J=7 Hz), 1.42-1.75 (4H, m)

ESI-MS: 561 [M+H]

### EXAMPLE 169

(3S)-3-[(2S)-5-Carboxy-2-[2-(carboxymethyl)benzoylamino]pentanoyl]oxy-4-(2-naphthyl)butanamide

NMR (DMSO-d₆, δ): 8.75 (1H, d, J=7 Hz), 7.81-7.92 (3H, m), 7.77 (1H, s), 7.22-7.54 (8H, m), 6.86 (1H, br s), 5.42 (1H, m), 4.30 (1H, m), 3.83 (1H, d, J=16 Hz), 3.71 (1H, d, J=16 Hz), 3.13 (1H, dd, J=14, 5 Hz), 3.03 (1H, dd, J=14, 6 Hz), 2.36 (2H, d, J=7 Hz), 2.13 (3H, t, J=7 Hz), 1.43-1.74 (4H, m)

ESI-MS: 535 [M+H]

### EXAMPLE 170

(3S)-3-[(2S)-5-Carboxy-2-[2-(methoxycarbonylmethyl)benzoylamino]pentanoyl]oxy-4-(2-naphthyl)butanamide

NMR (DMSO-d₆, δ): 8.71 (1H, d, J=7 Hz), 7.75-7.92 (4H, m), 7.28-7.54 (8H, m), 6.87 (1H, br s), 5.42 (1H, m), 4.28 (1H, m), 3.91 (1H, d, J=16 Hz), 3.81 (1H, d, J=16 Hz), 3.53 (3H, s), 3.13 (1H, dd, J=14, 5 Hz), 3.04 (1H, dd, J=14, 6 Hz), 2.36 (2H, d, J=7 Hz), 2.14 (3H, t, J=7 Hz), 1.42-1.74 (4H, m)

ESI-MS: 549 [M+H]

### EXAMPLE 171

(3S)-3-[(2S)-5-Carboxy-2-(3-quinolylcarbonylamino)pentanoyl]oxy-4-(2-naphthyl)butanamide

NMR (CDCl₃—CD₃OD, δ): 9.36 (1H, s), 8.72 (1H, s), 8.15 (1H, d, J=15 Hz), 7.94 (1H, d, J=15 Hz), 7.6-7.9 (6H, m), 7.3-7.45 (3H, m), 5.6 (1H, m), 4.62 (1H, m), 3.15 (2H, d, J=7 Hz), 2.4-2.6 (2H, m), 2.1-2.4 (2H, m), 1.75-1.9 (2H, m), 1.5-1.6 (2H, m)

### EXAMPLE 172

(3S)-3-[%(2S)-5-Carboxy-2-(isoquinolin-3-ylcarbonylamino)pentanoyl]oxy-4-(2-naphthyl)butanamide

NMR (CDCl₃, δ): 9.1 (1H, s), 8.7 (1H, d, J=15 Hz), 8.50 (1H, s), 7.9-8.05 (2H, m), 7.6-7.8 (5H, m), 7.25-4 (3H, m), 6.9 (1H, br s), 6.3 (1H, br s), 5.6 (1H, m), 4.8 (1H, m), 3.0-3.3 (2H, m), 2.4-2.6 (2H, m), 2.1-2.4 (2H, m), 1.75-1.9 (2H, m), 1.5-1.6 (2H, m)

FAB-MS 528 [M+H]

### EXAMPLE 173

(3S)-3-[(2S)-5-Carboxy-2-(isoquinolin-1-ylcarbonylamino)pentanoyl]oxy-4-(2-naphthyl)butanamide

NMR (CDCl₃—CD₃OD, δ): 9.45 (1H, d, J=10 Hz), 8.72 (1H, d, J=10 Hz), 8.4 (1H, d, J=7 Hz), 7.6-7.9 (11H, m), 7.0 (1H, br s), 6.4 (1H, br s), 5.6 (1H, m), 4.7 (1H, m), 3.0-3.3 (2H, m), 2.4-2.6 (2H, m), 2.1-2.4 (2H, m), 1.75-1.9 (2H, m), 1.5-1.6 (2H, m)

FAB-MS 528 [M+H]

### EXAMPLE 174

(3S)-3-[(2S)-2-(2-Benzylbenzoyl)amino-5-carboxypentanoyl]oxy-4-(2-naphthyl)butanamide

NMR (DMSO-d₆, δ): 8.75 (1H, d, J=10 Hz), 7.1-7.9 (17H, m), 6.88 (1H, br s), 5.4 (1H, m), 4.32 (1H, m), 4.1 (2H, ABq), 2.95-3.15 (2H, m), 2.32 (2H, d, J=7 Hz), 2.1 (2H, m), 1.4-1.7 (4H, m)

ESI-MS: 567 [M+H]

### EXAMPLE 175

(3S)-3-[(2S)-5-Carboxy-2-(2-naphthylcarbonylamino)pentanoyl]oxy-4-(2-naphthyl)butanamide

NMR (DMSO-d₆, δ): 8.9 (1H, d, J=10 Hz), 8.52 (1H, s), 7.35-8.1 (14H, m), 6.9 (1H, br s), 5.45 (1H, m), 4.44 (1H, m), 3.0-3.2 (2H, m), 2.35 (2H, d, J=7 Hz), 2.2 (2H, t, J=7 Hz), 1.7-1.85 (2H, m), 1.5-1.7 (2H, m)

ESI-MS: 527 [M+H]

### EXAMPLE 176

(3S)-3-[(2S)-2-Benzoylamino-5-carboxypentanoyl]oxy-4-(2-naphthyl)butanamide

NMR (DMSO-d₆, δ): 8.74 (1H, d, J=10 Hz), 7.3-8.0 (13H, m), 6.88 (1H, br s), 5.4 (1H, m), 4.4 (1H, m), 3.0-3.2 (2H, m), 2.32 (2H, d, J=7 Hz), 2.18 (2H, t, J=7 Hz), 1.4-1.8 (4H, m)

ESI-MS: 477 [M+H]

### EXAMPLE 177

(3S)-3-[(2S)-5-Carboxy-2-(2-phenethylbenzoylaminio)pentanoyl]oxy-4-(2-naphthyl)butanamide

NMR (CDCl₃, δ): 8.8 (1H, d, J=10 Hz), 7.1-7.9 (17H, m), 6.85 (1H, br s), 5.44 (1H, m), 4.36 (1H, m), 2.8-3.2 (6H, m), 2.35 (2H, d, J=7 Hz), 2.18 (2H, t, J=7 Hz), 1.45-1.8 (4H, m)

ESI-MS: 581 [M+H]

### EXAMPLE 178

(3S)-3-[(2S)-2-(3-Benzylbenzoyl)amino-5-carboxypentanoyl]oxy-4-(2-naphthyl)butanamide

NMR (DMSO-d₆, δ): 8.75 (1H, d, J=10 Hz), 7.1-7.9 (17H, m), 6.88 (1H, br s), 5.45 (1H, m), 4.4 (1H, m), 4.04 (2H, s), 2.95-3.15 (2H, m), 2.32 (2H, d, J=7 Hz), 2.2 (2H, t, J=7 Hz), 1.4-1.7 (4H, m)

ESI-MS 567 [M+H]

### EXAMPLE 179

(3S)-3-[(2S)-2-Benzylnaphthalen-2-ylcarbonyl)amino-5-carboxypentanoyl]oxy-4-(6-ethyl-2-naphthyl)butanamide

NMR (DMSO-d₆, δ): 8.82 (1H, d, J=10 Hz), 7.1-7.9 (18H, m), 8.86 (1H, br s), 5.4 (1H, m), 4.32 (1H, m), 4.26 (2H, ABq), 3.0-3.15 (2H, m), 2.72 (2H, q, J=7 Hz), 2.36 (2H, d, J=7 Hz), 2.12 (2H, t, J=7 Hz), 1.4-1.7 (4H, m), 1.22 (3H, t, J=7 Hz)

ESI-MS 645 [M+H]

### EXAMPLE 180

3-[N-Methyl-{(2S)-2-(3-benzylnaphthalen-2-ylcarbonyl)amino-5-carboxypentanoyl}amino]propanamide

ESI-MS: 488 [M−H]

mp: 147-157° C.

### EXAMPLE 181

(3S)-3-[N-(n-Pentyl)-{(2S)-2-(1-benzylindol-3-ylcarbonyl)amino-5-carboxypentanoyl}amino]dodecanamide

ESI-MS: 661 [M+H]

mp 125-127° C.

### EXAMPLE 182

(3S)-3-[N-(n-Pentyl)-{(2S)-2-(1-benzylindol-3-ylcarbonyl)amino-5-carboxypentanoyl}amino]nonanamide

ESI-MS: 619 [M+H]

mp: 127-129° C.

### EXAMPLE 183

(3S)-3-[N-(n-Butyl)-{(2S)-5-carboxy-2-[(1−benzylindol-3-ylcarbonyl)amino]pentanoyl}amino]nonanamide

ESI-MS: 605 [M+H]

mp 124-127° C.

### EXAMPLE 184

(3S)-3-[(N-(n-Butyl)-{(2S)-5-carboxy-2-[(1−(2-chlorobenzyl)indol-3-ylcarbonyl)amino]pentanoyl}amino]nonanamide

ESI-MS 639 [M+H]

mp: 153-156° C.

### EXAMPLE 185

(3S)-3-[N-(n-Propyl)-{(2S)-5-carboxy-2-[(1−(3-chlorobenzyl)indol-3-ylcarbonyl)amino]pentanoyl}amino]nonanamide

ESI-MS: 625 [M+H]

mp 106-109° C.

### EXAMPLE 186

(3S)-3-[N-(n-Propyl)-{(2S)-5-carboxy-2-[(1−(1-naphthylmethyl)indol-3-ylcarbonyl)amino]pentanoyl}amino]nonanamide

ESI-MS: 641 [M+H]

mp: 139-141° C.

### EXAMPLE 187

(3S)-3-[N-(n-Propyl)-{(2S)-5-carboxy-2-[(1-(2-pyridylmethyl)indol-3-ylcarbonyl)amino]pentanoyl}amino]nonanamide

ESI-MS: 592 [M+H]

mp 78-95° C.

### EXAMPLE 188

(3S)-3-[-N-(n-Propyl)-{(2S)-5-carboxy-2-[(1−(2-chlorobenzyl)indol-3-ylcarbonyl)amino]pentanoyl}amino]nonanamide

ESI-MS: 625 [M+H]

mp: 175-180° C.

### EXAMPLE 189

(3S)-3-[N-(n-Propyl)-{(2S)-2-(1-(2-chlorobenzyl)indol-3-ylcarbonyl)amino-5-carboxypentanoyl amino]heptanamide

ESI-MS: 597 [M+H]

mp 95-98° C.

### EXAMPLE 190

(3S)-3-[N-(n-Propyl)-{(2S)-2-(1-(1-naphthylmethyl)indol-3-ylcarbonyl)amino-5-carboxypentanoyl}amino]heptanamide

ESI-MS: 613 [M+H]

mp: 98-116° C.

### EXAMPLE 191

(3S)-3-[N-(n-Propyl)-{(2S)-5-carboxy-2-[(1-(2-pyridylmethyl)indol-3-ylcarbonyl)amino]pentanoyl}amino]-4-(6-ethyl-2-naphthyl)butanamide

ESI-MS: 676 [M+H]

mp: 110-116° C.

### EXAMPLE 192

(3S)-3-[N-(n-Propyl)-{(2S)-2-(1-benzylindol-3-ylcarbonyl)amino-5-carboxypentanoyl}amino]-4-(6-ethyl-2-naphthyl)butanamide

ESI-MS: 673 [M−H]

mp: 105-116° C.

### EXAMPLE 193

(3S)-3-[N-(n-Butyl)-{(2S)-2-(1-benzylindol-3-ylcarbonyl)amino-5-carboxypentanoyl}amino]-4-(6-ethyl-2-naphthyl)butanamide

ESI-MS: 689 [M+H]mp 100-116° C.

### EXAMPLE 194

(3S)-3-[N-(n-Butyl)-((2S)-2-(1−(2-chlorobenzyl)indol-3-ylcarbonyl)amino-5-carboxypentanoyl}amino]-4-(6-ethyl-2-naphthyl)butanamide

ESI-MS: 723 [M+H]

mp 100-116° C.

The following compounds (Examples 195 to 199) were obtained according to a similar manner to that of Example 33.

### EXAMPLE 195

(3S)-3-[(2S)-5-Carboxy-2-(tert-butoxycarbonylamino)pentanoyl]oxy-5-(n-decyloxy)pentanamide

NMR (CDCl₃, δ): 6.90 (1H, br s), 6.24 (1H, br s), 5.40 (1H, m), 5.20 (1H, d, J=8 Hz), 4.27 (1H, m), 3.32-3.57 (4H, m), 2.40-2.72 (2H, m), 2.30-2.40 (2H, m), 1.48-2.03 (8H, m), 1.44 (9H, s), 1.16-1.35 (14H, m), 0.87 (3H, t, J=7 Hz)

### EXAMPLE 196

(3S)-3-[(2S)-5-Carboxy-2-(tert-butoxycarbonylamino)pentanoyl]oxy-6-(n-nonyloxy)hexanamide

NMR (CDCl₃, δ): 6.77 (1H, br s), 6.15 (1H, br s), 5.33 (1H, m), 5.22 (1H, d, J=8 Hz), 4.26 (1H, m), 3.32-3.46 (4H, m), 2.45-2.56 (2H, m), 2.27-2.40 (2H, m), 1.47-1.93 (8H, m), 1.44 (9H, s), 1.16-1.36 (14H, m), 0.88 (3H, t, J=7 Hz)

### EXAMPLE 197

(3S)-3-[(2S)-5-Carboxy-2-(3-quinolylcarbonylamino)pentanoyl]oxy-10-phenyldecanamide

NMR (CDCl₃, δ): 9.34 (1H, s), 8.62 (1H, s), 7.55-8.25 (4H, m), 7.1-7.4 (11H, m), 6.85 (1H, br s), 6.40 (1H, br s), 5.35 (1H, m), 4.80 (1H, m), 2.4-2.7 (6H, m), 1.5-2.15 (8H, m), 1.2-1.4 (8H, m)

FAB-MS 562 [M+H]

### EXAMPLE 198

(3S)-3-[(2S)-(tert-Butoxycarbonyl)amino-5-carboxypentanoyl]oxy-10-phenyldecanamide

NMR (CDCl₃, δ): 7.1-7.3 (6H, m), 6.20 (1H, br s), 5.26 (1H, m), 5.20 (1H, d, J=15.0 Hz), 4.28 (1H, m), 2.3-2.65 (6H, m), 1.5-1.9 (8H, m), 1.45 (9H, s), 1.2-1.4 (8H, m)

### EXAMPLE 199

(3S)-3-[(2S)-2-(tert-Butoxycarbonyl)amino-5-carboxypentanoyl]oxy-4-(4-biphenylyl)butanamide

NMR (DMSO-d₆, δ): 7.3-7.7 (10H, m), 7.22 (1H, d, j=15.0 Hz), 6.84 (1H, br s), 5.32 (1H, m), 3.88 (1H, m), 2.8-3.0 (2H, m), 2.3 (2H, m), 2.14 (2H, m), 1.3-1.7 (13H, m)

FAB-MS: 499 [M+H]

### EXAMPLE 200

To a stirring solution of (3S)-3-[N-(n-propyl)-{(2S)-2-(1−benzylindol-3-ylcarbonyl)amino-5-methoxycarbonylpentanoyl}amino]dodecanamide (0.23 g) in methanol (4.5 ml) was added 1N sodium hydroxide (0.71 ml) at room temperature and allowed to stand overnight. The mixture was diluted with 1N hydrochloric acid (2 ml) and concentrated under reduced pressure. The residue was extracted with ethyl acetate and the organic layer was washed with water and brine. The organic layer was dried with magnesium sulfate and concentrated in vacuo. The residue was triturated with ethyl ether to give (3S)-3-[N-(n-propyl)-{(2S)-2-(1−benzylindol-3-ylcarbonyl)amino-5-carboxypentanoyl}amino]dodecanamide (114 mg).

ESI-MS: 633 [M+H]

mp: 155-160° C.

The following compounds (Examples 201 to 207) were obtained according to a similar manner to that of Example 200.

### EXAMPLE 201

(3S)-3-[N-(n-Propyl)-{(2S)-2-(1−(2-chlorobenzyl)indol-3-ylcarbonyl)amino-5-carboxypentanoyl}amino]dodecanamide

ESI-MS: 667 [M+H]

mp: 145-150° C.

### EXAMPLE 202

(3S)-3-[N-(n-Butyl)-{(2S)-5-carboxy-2-[(1−(2-chlorobenzyl)indol-3-ylcarbonyl)amino]pentanoyl}amino]-4-(4-n-heptylphenyl)butanamide

ESI-MS 743 [M+H]

mp: 79-81° C.

### EXAMPLE 203

(3S)-3-[(N-(n-Butyl)-{(2S)-5-carboxy-2-(2-quinolylcarbonylamino)pentanoyl}amino]-4-(4-n-heptylphenyl)butanamide

ESI-MS: 631 [M+H]

### EXAMPLE 204

(3S)-3-[N-Ethyl-{(2S)-2-(1-(2-chlorobenzyl)indol-3-ylcarbonyl)amino-5-carboxypentanoyl}amino]nonanamide

ESI-MS: 611 [M+H]

mp: 180-183° C.

### EXAMPLE 205

(3S)-3-[N-Ethyl-{(2S)-2-(1-benzylindol-3-ylcarbonyl)amino-5-carboxypentanoyl}amino]nonanamide

ESI-MS: 577 [M+H]

mp: 80-85° C.

### EXAMPLE 206

(3S)-3-[N-(n-Butyl)-{(2S)-2-(1-(2-chlorobenzyl)indol-3-ylcarbonyl)amino-5-carboxypentanoyl}amino]heptanamide

ESI-MS: 611 [M+H]

mp: 105-110° C.

### EXAMPLE 207

(3S)-3-[N-(n-Butyl)-{(2S)-2-(1-(1-naphthylmethyl)indol-3-ylcarbonyl)amino-5-carboxypentanoyl}amino]heptanamide

ESI-MS: 627 [M+H]

mp: 105-113° C.

### EXAMPLE 208

(3S)-3-[N-Ethyl-{(2S)-5-carboxy-2-(2-quinolylcarbonylamino)pentanoyl}amino]hexadecanamide (300 mg) was dissolved in 4N hydrogen chloride in ethyl acetate (2 ml) at room temperature. After being stirred at the same temperature for 10 minutes, the solvent was removed under reduced pressure and the resulting solid was triturated with ethyl acetate to give (3S)-3-[(N-ethyl-{(2S)-5-carboxy-2-(2-quinolylcarbonylamino)pentanoyl}amino]hexadecanamide hydrochloride (194 mg).

### EXAMPLE 209

(3S)-3-[N-(n-Butyl)-{(2S)-5-carboxy-2-(2-quinolylcarbonylamino)pentanoyl}amino]hexadecanamide hydrochloride was obtained according to a similar manner to that of Example 208.

### EXAMPLE 210

(3S)-3-[N-(n-Propyl)-{(2S)-2-(1−benzylindol-3-ylcarbonyl)amino-5-carboxypentanoyl}amino]nonanamide was obtained according to a similar manner to that of Example 21.

ESI-MS: 591 [M+H]

mp 147-157° C.

1. A fatty acid derivative represented by the following formula 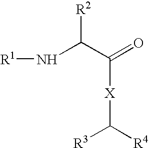
wherein
R¹ is acyl group,
R² is acyl(lower)alkyl,
R³ is hydrogen, aryl(lower)alkyl which may have one or more suitable substituent(s), aryl(higher)alkyl which may have one or more suitable substituent(s), heterocyclic(lower)alkyl which may have one or more suitable substituent(s), higher alkoxy(lower)alkyl, lower alkyl, or higher alkyl,
R⁴ is acyl(lower)alkyl, and
X is —O—, —NH— or 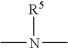
[wherein R⁵ is lower alkyl, [cyclo(lower)alkyl](lower)alkyl, aryl(lower)alkyl, or heterocyclic(lower)alkyl],
with proviso that X is 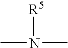
(wherein R⁵ is as defined above), when R³ is lower alkyl or higher alkyl, and a pharmaceutically acceptable salt thereof.

2. A compound of claim 1, wherein
R¹ is protected carboxy;
aryl(lower)alkanoyl which may have 1 to 3 suitable substituent(s) selected from the group consisting of lower alkoxy, aryl, carboxy(lower)alkyl, protected carboxy(lower)alkyl which may be substituted by aryl, protected carboxy(lower)alkenyl, amidated carboxy(lower)alkyl, and aryl(lower)alkyl which may have 1 to 3 suitable substituent(s) selected from the group consisting of lower alkyl, higher alkyl, lower alkoxy, aryl and halogen;
heterocyclic(lower)alkanoyl which may have 1 to 3 suitable substituent(s) selected from the group consisting of lower alkyl, aryl(lower)alkyl which may have 1 to 3 suitable substituent(s) selected from the group consisting of lower alkyl, higher alkyl, lower alkoxy, aryl and halogen, and heterocyclic(lower)alkyl which may have 1 to 3 suitable substituent(s) selected from the group consisting of lower alkyl, higher alkyl, lower alkoxy, aryl and halogen;
R² is carboxy(lower)alkyl or protected carboxy(lower)alkyl,
R³ is hydrogen;
aryl(lower)alkyl which may have 1 to 3 suitable substituent(s) selected from the group consisting of lower alkyl, higher alkyl, lower alkoxy, aryl and halogen;
aryl(higher)alkyl which may have 1 to 3 suitable substituent(s) selected from the group consisting of lower alkyl, higher alkyl, lower alkoxy, aryl and halogen;
heterocyclic(lower)alkyl which may have 1 to 3 suitable substituent(s) selected from the group consisting of lower alkyl, higher alkyl, lower alkoxy, aryl and halogen;
higher alkoxy(lower)alkyl;
lower alkyl; or
higher alkyl,
R⁴ is carbamoyl(lower)alkyl, and X is —O—, —NH— or 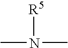
[wherein R⁵ is lower alkyl, [cyclo(lower)alkyl]-(lower)alkyl, aryl(lower)alkyl, or heterocyclic(lower)alkyl],
with proviso that X is 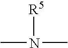
(wherein R⁵ is as defined above), when R³ is lower alkyl or higher alkyl.

3. A compound of claim 2, wherein
R¹ is lower alkoxycarbonyl;
phenyl(lower)alkanoyl or naphthyl(lower)alkanoyl, each of which may have 1 to 3 suitable substituent(s) selected from the group consisting of carboxy(lower)alkyl, lower alkoxycarbonyl(lower)alkyl which may be substituted by phenyl, lower alkoxycarbonyl(lower)alkenyl, carbamoyl(lower)alkyl and phenyl(lower)alkyl; or
heterocyclic(lower)alkanoyl which may have 1 to 3 suitable substituent(s) selected from the group consisting of pyridyl(lower)alkyl, naphthyl(lower)alkyl and phenyl(lower)alkyl which may have 1 to 3 suitable substituent(s) selected from the group consisting of lower alkyl and halogen, in which the heterocyclic moiety is unsaturated condensed heterocyclic group containing 1 to 4 nitrogen atom(s),
R² is carboxy(lower)alkyl or esterified carboxy(lower)alkyl,
R³ is hydrogen;
phenyl(lower)alkyl which may have 1 to 3 suitable substituent(s) selected from the group consisting of lower alkyl, higher alkyl and phenyl;
naphthyl(lower)alkyl which may be substituted by lower alkyl;
phenyl(higher)alkyl;
heterocyclic(lower)alkyl, in which the heterocyclic moiety is unsaturated condensed heterocyclic group containing 1 to 2 oxygen atom(s);
higher alkoxy(lower)alkyl;
lower alkyl; or
higher alkyl,
R⁴ is carbamoyl(lower)alkyl, and
X is —O—, —NH— or 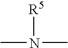
[wherein R⁵ is lower alkyl, phenyl(lower)alkyl, or pyridyl (lower) alkyl],
with proviso that X is 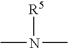
(wherein R⁵ is as defined above), when R³ is lower alkyl or higher alkyl.

4. A compound of claim 3, wherein
R¹ is lower alkoxycarbonyl;
phenyl(lower)alkanoyl or naphthyl(lower)alkanoyl, each of which may have carboxy(lower)alkyl, lower alkoxycarbonyl(lower)alkyl which may be substituted by phenyl, lower alkoxycarbonyl(lower)alkenyl, carbamoyl(lower)alkyl or phenyl(lower)alkyl;
heterocyclic(lower)alkanoyl which may have pyridyl(lower)alkyl, naphthyl(lower)alkyl or phenyl(lower)alkyl which may have 1 to 3 suitable substituent(s) selected from the group consisting of lower alkyl and halogen, in which the heterocyclic moiety is indolyl, quinolyl or isoquinolyl,
R² is carboxy(lower)alkyl, lower alkoxycarbonyl(lower)alkyl, or phenyl(lower)alkoxycarbonyl(lower)alkyl,
R³ is hydrogen;
phenyl(lower)alkyl which may have lower alkyl, (C₇-C₁₆)alkyl or phenyl;
naphthyl(lower)alkyl which may have lower alkyl;
phenyl (C₇-C₁₆) alkyl;
benzofuranyl(lower)alkyl;
(C₇-C₁₆)alkoxy(lower)alkyl;
lower alkyl; or
(C₇-C₁₆) alkyl,
R⁴ is carbamoyl(lower)alkyl, and
X is —O—, —NH— or 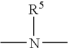
[wherein R⁵ is lower alkyl, phenyl(lower)alkyl, or pyridyl(lower)alkyl],
with proviso that X is 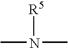
(wherein R⁵ is as defined above), when R³ is lower alkyl or (C₇-C₁₆)alkyl.

5. A compound of claim 4, wherein
R¹ is (C₁-C₄)alkoxycarbonyl;
phenyl(C₁-C₄)alkanoyl or naphthyl(C₁-C₄)alkanoyl, each of which may have carboxy(C₁-C₄)alkyl, (C₁-C₄)alkoxycarbonyl(C₁-C₄)alkyl which may be substituted by phenyl, (C₁-C₄)alkoxycarbonyl-C₂-C₄)alkenyl, carbamoyl(C₁-C₄)alkyl or phenyl(C₁-C₄)alkyl;
heterocyclic(C₁-C₄)alkanoyl which may have pyridyl(C₁-C₄)alkyl, naphthyl(C₁-C₄)alkyl or phenyl(C₁-C₄)alkyl which may have 1 to 3 suitable substituent(s) selected from the group consisting of (C₁-C₄)alkyl and halogen, in which the heterocyclic moiety is indolyl, quinolyl or isoquinolyl,
R² is carboxy(C₁-C₄)alkyl, methoxycarbonyl(C₁-C₄)alkyl, or benzyloxycarbonyl(C₁-C₄)alkyl,
R³ is hydrogen;
phenyl(C₁-C₄)alkyl which may have (C₁-C₄)alkyl, (C₇-C₁₆)alkyl or phenyl;
naphthyl(C₁-C₄)alkyl which may have (C₁-C₄)alkyl;
phenyl (C₇-C₁₆) alkyl;
benzofuranyl(C₁-C₄)alkyl;
(C₇-C₁₆)alkoxy(C₁-C₄)alkyl;
(C₃-C₆)alkyl; or
(C₇-C₁₆)alkyl,
R⁴ is carbamoyl(C₁-C₄)alkyl, and
X is —O—, —NH— or 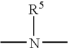
[wherein R⁵ is (C₁-C₅)alkyl, phenyl(C₁-C₄)alkyl, or pyridyl(C₁-C₄)alkyl],
with proviso that X is 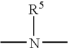
(wherein R⁵ is as defined above), when R³ is (C₃-C₆)alkyl or (C₇-C₁₆)alkyl.

6. A compound of claim 4, wherein
R¹ is indolyl(lower)alkanoyl which may have a suitable substituent selected from the group consisting of pyridyl (lower) alkyl, naphthyl (lower) alkyl, phenyl(lower)alkyl, lower alkylphenyl(lower)alkyl, and halophenyl(lower)alkyl,
R² is carboxy(lower)alkyl,
R³ is lower alkyl or (C₇-C₁₆)alkyl,
R⁴ is carbamoyl(lower)alkyl, and
X is 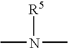
[wherein R⁵ is lower alkyl].

7. A compound of claim 6, which is selected from the group consisting of
(1) (3S)-3-[N-(n-Propyl)-{(2S)-5-carboxy-2-[(1−(2-chlorobenzyl)indol-3-ylcarbonyl)amino]pentanoyl}-amino]nonanamide,
(2) (3S)-3-[N-(n-Propyl)-{(2S)-2-(1−(2-chlorobenzyl)indol-3-ylcarbonyl)amino-5-carboxypentanoyl}amino]heptanamide,
(3) (3S)-3-[N-(n-Propyl)-{(2S)-2-(1−benzylindol-3-ylcarbonyl)amino-5-carboxypentanoyl}amino]-dodecanamide,
(4) (3S)-3-[N-(n-Propyl)-{(2S)-2-(1−(2-chlorobenzyl)indol-3-ylcarbonyl)amino-5-carboxypentanoyl}amino]dodecanamide,
(5) (3S)-3-[N-Ethyl-{(2S)-2-(1−(2-chlorobenzyl)indol-3-ylcarbonyl)amino-5-carboxypentanoyl}amino]-nonanamide,
(6) (3S)-3-[N-Ethyl-{(2S)-2-(1−benzylindol-3-ylcarbonyl)amino-5-carboxypentanoyl}amino]-nonanamide,
(7) (3S)-3-[N-(n-Butyl)-{(2S)-2-(1−(1-naphthylmethyl)indol-3-ylcarbonyl)amino-5-carboxypentanoyl}amino]-heptanamide, and
(8) (3S)-3-[N-(n-Propyl)-{(2S)-2-(1−benzylindol-3-ylcarbonyl)amino-5-carboxypentanoyl}amino]-nonanamide,
or a pharmaceutically acceptable salt thereof.

8. A process for preparing a compound of the formula 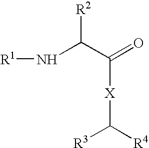
wherein
R¹ is acyl group,
R² is acyl(lower)alkyl,
R³ is hydrogen, aryl(lower)alkyl which may have one or more suitable substituent(s), aryl(higher)alkyl which may have one or more suitable substituent(s), heterocyclic(lower)alkyl which may have one or more suitable substituent(s), higher alkoxy(lower)alkyl, lower alkyl, or higher alkyl,
R⁴ is acyl(lower)alkyl, and
X is —O—, —NH— or 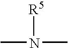
[wherein R⁵ is lower alkyl, [cyclo(lower)alkyl](lower)alkyl, aryl(lower)alkyl, or heterocyclic(lower)alkyl],
with proviso that X is 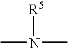
(wherein R⁵ is as defined above), when R³ is lower alkyl or higher alkyl, or a salt thereof, which comprises
1) reacting the compound of the formula 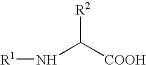
wherein R¹ and R² are each as defined above, or a reactive derivative at the carboxy group or a salt thereof, with the compound of the formula: 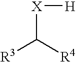
wherein R³, R⁴ and X are each as defined above, or a salt thereof,
2) reacting the compound of the formula: 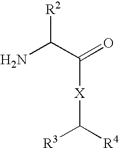
wherein R², R³, R⁴ and X are each as defined above, or a reactive derivative at the amino group or a salt thereof, with the compound of the formula:
R¹—OH
wherein R¹ is as defined above, or a reactive derivative or a salt thereof,
3) subjecting the compound of the formula: 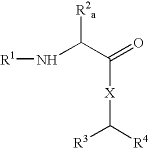
wherein R¹, R³, R⁴ and X are each as defined above, and
Rₐ² is protected carboxy(lower)alkyl, or a salt thereof, to elimination reaction of carboxy protective group, to give the compound of the formula: 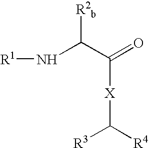
wherein R¹, R³, R⁴ and X are each as defined above, and
R_{b}² is carboxy(lower)alkyl,
or a salt thereof.

9. A pharmaceutical composition which comprises, as an active ingredient, a fatty acid derivative of claim 1 or a pharmaceutically acceptable salt thereof in admixture with pharmaceutically acceptable carriers or excipients.

10. Use of a fatty acid derivative of claim 1 or a pharmaceutically acceptable salt thereof for the manufacture of a medicament.

11. A fatty acid derivative of claim 1 or a pharmaceutically acceptable salt thereof for use as a medicament.

12. A method for the prevention and/or the treatment of pancreatitis, hepatitis, chronic renal failure, shock, arthritis, respiratory disease, heart disease, allergic disease, thrombosis, arteriosclerosis, pain, autoimmune disease, dermal disease, inflammatory bowel disease, ophthalmic disease, nasal diseases, gout, trauma induced inflammation or liver diseases, which comprises administering a fatty acid derivative of claim 1 or a pharmaceutically acceptable salt thereof to a human being or an animal.
